(19) United States
(12) Patent Application Publication
(10) Pub. No.: US 2024/0252686 A1
(43) Pub. Date: Aug. 1, 2024

(54) AAV FOR THE GENE THERAPY OF WET-AMD

(71) Applicant: Skyline Therapeutics Limited, Cayman Islands (GB)

(72) Inventors: Jinzhao HOU, Shanghai (CN); Yanqun SHU, Shanghai (CN); Qiongqiong REN, Shanghai (CN); Heping LI, Shanghai (CN)

(73) Assignee: Skyline Therapeutics Limited, Cayman Islands (GB)

(21) Appl. No.: 18/606,573

(22) Filed: Mar. 15, 2024

Related U.S. Application Data
(63) Continuation of application No. PCT/CN2022/119238, filed on Sep. 16, 2022.

(30) Foreign Application Priority Data
Sep. 18, 2021 (WO) PCT/CN2021/119223

(51) Int. Cl. A61K 48/00; A61P 27/02; C07K 16/22; C12N 15/86

(52) U.S. Cl. CPC A61K 48/0058; A61K 48/0075; A61P 27/02; C07K 16/22; C12N 15/86; C07K 2317/35; C07K 2317/569; C07K 2317/76; C07K 2317/92; C12N 2750/14122; C12N 2750/14143; C12N 2830/42

(57) ABSTRACT

The present invention provides a polynucleotide and vector, in particular AAV vector, encoding a nanobody against VEGF. The present invention also provides a method of treating a disease associated with VEGF, such as the overexpression of VEGF, e.g., Wet-AMD and DME comprising administering the vector by, e.g., intravitreal injection to provide stable expression of the nanobody in eyes.

### CROSS-REFERENCE TO RELATED APPLICATIONS

This application is a Continuation of PCT/CN2022/119238, filed Sep. 16, 2022, which claims priority to PCT/CN2021/119223, filed Sep. 18, 2021.

### SEQUENCE LISTING

The instant application contains a Sequence Listing which has been submitted in XML format and is hereby incorporated by reference in its entirety. Said XML copy, created on Mar. 14, 2024, is named P2022TC2187.xml and is 83,115 bytes.

### TECHNICAL FIELD

The present invention refers to gene therapy. In particular, the present invention involves the AAV vector for the gene therapy of Wet-AMD.

### BACKGROUND

Age-related macular degeneration is a clinical term that describes a variety of diseases that are characterized by the progressive loss of central vision. AMD is the leading cause of vision loss in aged individuals in many industrialized countries. Vision loss occurs due to the progressive degeneration of the macula, the region at the back of the eye comprising a high density of cone photoreceptors, which is specialized for high-acuity, central vision.

AMD can manifest as dry (non-neovascular) AMD and wet AMD. Dry AMD is the more common (85-90% of cases) and milder form of AMD, and is characterized by small, round, white-yellow lesions (drusen) in and under the macula. As the eye ages, debris from the photoreceptors and surrounding tissues accumulates within and above the Bruch's membrane (dry AMD), which causes inflammation and recruitment of inflammatory cells to the retina. These cells and retinal pigment epithelium (RPE) produce cytokines including VEGF that stimulates the blood vessel to grow. At this stage the dry AMD progresses into wet AMD. The blood and fluid from the leaky blood vessel damage photoreceptor and the nerves that are required for vision, which could result in permanent vision loss if untreated (wet AMD).

There are some commercially available medicaments for the treatment of wet AMD, such as Bevacizumab (Avastin), Conbercept, and Aflibercept. However, these medicaments need to be administered at regular intervals, and are costly.

A humanized nanobody (Nb24) against vascular endothelial growth factor (VEGF) has been reported to be effective for treating wet AMD (CN110452297B). But there is still a need of a method that can achieve desired effect on the treatment of wet AMD without repeated administration of medicaments, preferably by the administration of limited doses, more preferably a single dose.

### SUMMARY

In a first aspect, the present invention provides a polynucleotide construct comprising a first expression cassette comprising a first nucleotide sequence selected from SEQ ID NOs: 1, 2, 3, 4, 5, 6, 7, 8, 9, 10 and 11 operably linked to a first promoter. In some embodiments, the first nucleotide sequence is selected from SEQ ID NOs: 4, 5 and 7. In some embodiments, the first nucleotide sequence is SEQ ID NO: 5.

In some embodiments, the first expression cassette comprises a second nucleotide sequence linked to the first nucleotide sequence via a linker. In some embodiments, the second nucleotide sequence is selected from SEQ ID NOs: 12, 13, 14, 15, 16, 17, 18, 19, 20, 21 and 22. In some embodiments, the second nucleotide sequence is selected from ID NOs: 15, 16 and 18.

In some embodiments, the first promoter is a chicken beta-actin promoter or a promoter from CMV. In some embodiments, the first promoter comprises a nucleotide sequence of SEQ ID NO: 26 or 27.

In some embodiments, the first expression cassette comprises a first enhancer. Preferably, the first enhancer is upstream of the first promoter. In some embodiments, the first enhancer is a cytomegalovirus (CMV) early enhancer. In some embodiments, the first enhancer comprises SEQ ID NO: 25.

In some embodiments, the first expression cassette comprises a first polyadenylation signal sequence downstream of the coding sequence. In some embodiments, the first polyadenylation signal sequence is selected from SEQ ID NOs: 31 and 32.

In some embodiments, the first expression cassette comprises a first intron. Preferably, the first intron is upstream of the first nucleotide sequence. In some embodiments, the first intron is at least 200 nucleotides in length. In some embodiments, the first intron comprises SEQ ID NO: 28.

In some embodiments, the polynucleotide construct further comprises a second expression cassette comprising a third nucleotide sequence selected from SEQ ID NOs: 1, 2, 3, 4, 5, 6, 7, 8, 9, 10 and 11 operably linked to a second promoter. In some embodiments, the third nucleotide sequence is selected from SEQ ID NOs: 4, 5 and 7. In some embodiments, the third nucleotide sequence is SEQ ID NO: 5.

In some embodiments, the second promoter is a chicken beta-actin promoter or a promoter from CMV. In some embodiments, the second promoter comprises a nucleotide sequence of SEQ ID NO: 26 or 27.

In some embodiments, the second expression cassette comprises a second enhancer, preferably upstream of the second promoter. In some embodiments, the second enhancer is a CMV early enhancer. In some embodiments, the second enhancer comprises SEQ ID NO: 25.

In some embodiments, the second expression cassette comprises a second polyadenylation signal sequence downstream of the coding sequence. In some embodiments, the second polyadenylation signal sequence is selected from SEQ ID NOs: 31 and 32.

In some embodiments, the second expression cassette comprises a second intron, preferably upstream of the third nucleotide sequence. In some embodiments, the second intron is at least 200 nucleotides in length. In some embodiments, the second intron comprises SEQ ID NO: 28.

In some embodiments, the construct comprises the genome of a recombinant AAV. In some embodiments, the construct comprises 5′ and 3′ inverted terminal repeats (ITRs) of adeno-associated virus (AAV). In some embodiments, the 5′ and 3′ ITRs are AAV ITR130 and/or AAV ITR 105. In some embodiments, both the 5′ and 3′ ITRs are AAV ITR130.

In a second aspect, the present invention provides a recombinant adeno-associated virus (rAAV) comprising a genome comprising the polynucleotide construct of the present invention. The present invention further provides a host cell comprising the polynucleotide construct or the AAV of the present invention.

In a third aspect, the present invention provides a pharmaceutical composition for preventing or treating a disease associated with VEGF. In some embodiments, the composition comprises the rAAV of the present invention.

In a fourth aspect, the present invention provides a method of preventing or treating a disease associated with VEGF, comprising administering the rAAV or the pharmaceutical composition of the present invention to a subject in need thereof. The present invention further provides use of the polynucleotide construct, the rAAV or the pharmaceutical of the present invention in the preparation of a medicament for preventing or treating a disease associated with VEGF. In some embodiments, the disease is Wet-AMD or Diabetic macular edema (DME). In some embodiments, the rAAV, the pharmaceutical composition or the medicament is administered by intravitreal injection.

### BRIEF DESCRIPTION OF THE DRAWINGS

FIG. 1 shows the map of an exemplified plasmid with a pGCB108 backbone.

FIGS. 2A-2G show the structure of the constructs expressing codon optimized sequence encoding the nanobody with pelB signal sequence. FIG. 2A shows the structure of the constructs an expressing mono-valent nanobody including 11 constructs expressing the humanized nanobody HuNb24 (FIG. 2A). FIGS. 2B-2G show the structure of the constructs expressing bi-valent Nb24. The element “CMVen” represents cytomegalovirus (CMV) early enhancer element (SEQ ID NO: 25); the element “CB promoter” represents the chicken beta-actin promoter (SEQ ID NO: 26); the element “hGbin intron” represents the first exon and the first intron of chicken beta-actin gene (SEQ ID NO: 28), the element “CoHuNb24-n” represents a codon optimized sequence encoding Nb24 nanobody (selected from SEQ ID NOs: 1, 2, 3, 4, 5, 6, 7, 8, 9, 10 and 11), the element “HA” represent the sequence encoding an HA tag (SEQ ID NO: 30); the element “rGB poly(A) signal” represents the polyadenylation signal sequence from rabbit globin gene (SEQ ID NO: 31); the element “CBA promoter” represents a “CMVen” and a “CB promoter”; the element “(G4S)n” represents the coding sequence for a linker selected from (G4S)4, (G4S)3, (G4S)2 and G4S (SEQ ID NOs: 36, 37, 38 and 39, respectively); the element “hGH poly(A)” represents the polyadenylation signal sequence from human growth factor gene (SEQ ID NO: 32); the element “Co3.1HuNb24” represents a codon optimized sequence encoding Nb24 nanobody (SEQ ID NO: 40); and the element “hlgG1Fc” represents a sequence encoding human IgG1 Fc region (SEQ ID NO: 42). The elements with the same name in the following Figures represent the same sequences.

FIG. 3 shows the structure of a constructs expressing a benchmark gene, Beovu-scFv (SEQ ID NO: 43).

FIGS. 4A-4C show the structure of the constructs expressing Nb24 with a different signal sequence (“IL2signal”, SEQ ID NO: 41), and optionally, a human IgG1 Fc region.

FIG. 5 shows the structure of the construct comprising two cassettes expressing Nb24. The element “CMV promoter” represents a “CMVen” and a promoter from CMV (SEQ ID NO: 44).

FIG. 6 shows the structure of the construct expressing Nb24 with an altered intron (“pCI intron”, SEQ ID NO: 29).

FIGS. 7A-7B show the detection of the expression of mono-valent Nb24 by Western blotting. FIG. 7A shows the image of the membrane, in which lanes indicated by numbers (1-11) correspond to the supernatants from the cells transfected with the plasmids pGCB108-ITR130-AMDF1Nb24-n (n=1, 2, 3, 4, 5, 6, 7, 8, 9, 10 or 11), respectively. The lane “P” is positive control, and the lane “sup” corresponds to the supernatant from non-transfected cells. FIG. 7B shows the quantification of the Western blotting, normalized to the expression level of Nb24-11 (the plasmid comprising SEQ ID NO: 11), which is defined as “1”.

FIG. 8 shows the comparison of the expressions of mono- and bi-valent Nb24s by Western blotting. The lanes indicated by numbers (1-8) correspond to the supernatants from the cells transfected with the plasmids for expressing mono- and bi-valent Nb24s, and the lane “sup” corresponds to the supernatant from non-transfected cells.
Lanes indicated by 1: pGCB108-ITR130-AMDF1 Bi-huNb24-pCDNA3.1+-HA_IL2sig;
Lanes indicated by 2 or 6: pGCB108-ITR130-AMDF1 Bi-huNb24-hlgG1Fc;
Lanes indicated by 3: pGCB108-ITR130-AMDF1Co3.1Nb24(IL2 signal)-HA;
Lanes indicated by 4: pGCB108-ITR130-AMDF1Co3.1Nb24(IL2 signal)-hlgG1Fc;
Lanes indicated by 5: pGCB108-ITR130-AMDF1Nb24-4(IL2 signal)-HA;
Lanes indicated by 7: pGCB108-ITR130-AMDF1Nb24-2; and
Lanes indicated by 8: pGCB108-ITR130-AMDF1Nb24-4.

FIG. 9 shows the detection of the expression with plasmids constructed with various strategies by Western blotting. The lanes correspond to the plasmids as follows.
Lanes 1 and 7: pGCB108-ITR130-AMDF1-Beovu-scFv;
Lane 2: pGCB108-ITR130-AMDF1 Bi-huNb24-pCDNA3.1+-HA_IL2sig;
Lane 3: pGCB108-ITR130-AMDF1Nb24-5-HA-CMV-AMDF1-Nb24-5-HA;
Lane 4: pCB108-5ITR130-CBA-pCI-Nb24-5-hGHpA;
Lane 5: pGCB108-ITR130-AMDF1Nb24-5-(G4S)4-Nb24-5-HA
Lane 6: pGCB108-ITR130-AMDF1huNb24-5-hGHpA;
Lane 8: pGCB108-ITR130-AMDF1Nb24-5; and
Lane 9: negative control.

FIG. 10 shows the detection of the expression with plasmids pGCB108-ITR130-AMDF1huNb24-5-hGHpA (lane 1) and pGCB108-ITR130-AMDF1Nb24-5-(G4S)n-Nb24-5-HA (n=1-4, lanes 2-5, respectively, constructs #36-33) by Western blotting.

FIG. 11 shows the detection of the expression of various versions of Nb24. The lanes indicated by “F1F3” correspond to the cell transfected with the plasmid pGCB108-ITR130-AMDF1-Beovu-scFv; the lanes indicated by “G4S4” correspond to the cell transfected by the plasmid pGCB108-ITR130-AMDF1Nb24-5-(G4S)4-Nb24-5-HA; the lanes indicated by “Codon3.1” correspond to the cell transfected with the plasmid pGCB108-ITR130-AMDF1 Bi-huNb24-pCDNA3.1+-HA_IL2sig; the lanes indicated by “CBA-CMV” correspond to the cell transfected with the plasmid pGCB108-ITR130-AMDF1Nb24-5-HA-CMV-AMDF1-Nb24-5-HA; and the lanes indicated by “Nb24-5” correspond to the cell transfected with the plasmid pGCB108-ITR130-AMDF1huNb24-5-hGHpA.

FIGS. 12A-12D show the binding of antibodies to VEGF. In FIG. 12A, “HuNb24-5-HA” represents the product expressed by pGCB108-ITR130-AMDF1huNb24-5-hGHpA and “Beovu-scFv-HA” represents the product expressed by pGCB108-ITR130-AMDF1-Beovu-scFv, OD525 represents the plate background. In FIG. 12B, “CMV-Nb24-5-HA” represents the product expressed by pGCB108-ITR130-AMDF1Nb24-5-HA-CMV-AMDF1-Nb24-5-HA; “G4S4-Nb24-5-HA” represents the product expressed by pGCB108-ITR130-AMDF1Nb24-5-(G4S)4-Nb24-5-HA; “Nb24-5-HA” represents the product expressed by pGCB108-ITR130-AMDF1huNb24-5-hGHpA; “F1F3-HA” represents the product expressed by pGCB108-ITR130-AMDF1-Beovu-scFv. In FIG. 12C, Plots 1-4 represent the products expressed by pGCB108-ITR130-AMDF1Nb24-5-(G4S)n-Nb24-5-HA (n=1-4), respectively; Plot 5 represents the product expressed by pGCB108-ITR130-AMDF1Nb24-4-(G4S)4-Nb24-7-HA; and plot 6 represents the product expressed by pGCB108-ITR130-AMDF1Nb24-4-(G4S)4-Nb24-4-HA. In FIG. 12D, “Beovu-scFv-HA” represents the product expressed by pGCB108-ITR130-AMDF1-Beovu-scFv; “Nb24-5” represents the product expressed by pGCB108-ITR130-AMDF1huNb24-5-hGHpA; “Nb24-4-G4S4-Nb24-7” represents the product expressed by pGCB108-ITR130-AMDF1Nb24-4-(G4S)4-Nb24-7-HA; “Nb24-7-G4S4-Nb24-7” represents the product expressed by pGCB108-ITR130-AMDF1Nb24-7-(G4S)4-Nb24-7-HA; and “Codon3.1Nb24-G4S4-Codon3.1Nb24” represents the product expressed by pGCB108-ITR130-AMDF1 Bi-huNb24-pCDNA3.1+-HA_IL2sig.

FIGS. 13A-13B show the inhibition of Nb24 to the VEGFR2. FIG. 13A shows the inhibition of the bi-valent Nb24s, and FIG. 13B shows the comparison between mono-bivalent and bi-valent Nb24s.

FIG. 14A shows the binding of VEGFR2 to VEGF, and FIG. 14B shows the inhibition of antibodies against VEGF to the binding.

FIG. 15A shows the dose curve of the binding of VEGF to VEGFR2, and FIG. 15B shows the dose-dependent inhibition of bi-valent Nb24 to the binding.

FIGS. 16A-16B show the maps of the helper plasmid (FIG. 16A) and packaging plasmid (FIG. 16B) for the preparation of rAAV.

FIGS. 17A-17H show the effect of the AAVs administered to the Wet AMD model (Dutch belted rabbit). The area with fluorescence leakage shows the severity of retinal neovascularization. The animal for the left panel of FIG. 17D had suffered diarrhea since D7, so that was checked on D9 and dead on D11. Autopsy did not find other changes of pathophysiology.

FIG. 18 shows the ELISA evaluating the inhibition of Nb24s to VEGFR2.

FIG. 19 shows the ELISA evaluating the binding of bi-valent Nb24s to VEGF, “24-5-G4Sn-24-5” (n=1-4), “24-4-G4S4-24-7” and “24-4-G4S4-24-7” represent the products expressed by plasmids pGCB108-ITR130-AMDF1Nb24-5-(G4S)n-Nb24-5-HA (n=1-4), pGCB108-ITR130-AMDF1Nb24-4-(G4S)4-Nb24-4-HA, and pGCB108-ITR130-AMDF1Nb24-4-(G4S)4-Nb24-7-HA, respectively.

FIGS. 20 and 21 show the denaturing electrophoresis of rAAV genomes.

FIG. 22 shows the ELISA evaluating the binding of bi-valent Nb24 to VEGF, “24-5-G4S2-24-5”, “24-5-G4S2-24-7” and “24-7-G4S2-24-5” represent the products expressed by plasmids pGCB108-ITR130-AMDF1Nb24-5-(G4S)2-Nb24-5-HA, pGCB108-ITR130-AMDF1Nb24-5-(G4S)2-Nb24-7-HA, and pGCB108-ITR130-AMDF1Nb24-7-(G4S)2-Nb24-5-HA, respectively.

### DETAILED DESCRIPTION OF THE INVENTION

### 1. Definition

Unless otherwise indicated, all terms used herein have the same meaning as they would to one skilled in the art, and the practice of the present invention will employ conventional techniques of microbiology and recombinant DNA technology, which are within the knowledge of those of skill in the art.

As used herein, “Nb24” refers to a humanized nanobody against VEGF, which is identified by Novamab, with an amino acid sequence of SEQ ID NO: 23, and the coding sequence thereof. “Nb24-n” refers to the codon optimized versions of the coding sequence, or the expression product of the same.

As used herein, the term “nanobody”, also referred to as “single domain antibody”, is an antibody that naturally lacks light chain, which is first discovered in the peripheral blood of alpaca. A nanobody contains only one heavy chain variable region (VHH) and two conventional CH2 and CH3 regions. The individually cloned and expressed VHH is stable in structure and has the antigen binding activity comparable to that of the original heavy chain antibody, and is the smallest unit known to bind the target antigen. In the present invention, the term “nanobody” encompasses the VHH thereof. The VHH crystal is 2.5 nm, the length is 4 nm, and the molecular weight is only 15KDa.

Adeno-associated virus (AAV) is a member of Parvoviridae family. It is a simple single-stranded DNA virus, and requires a helper virus (such as adenovirus) for replication. The genome of a wildtype AAV contains approximately 4.7 kilobases (kb), comprising the cap and rep genes between two inverted terminal repeat (ITR) sequences, approximately 145 nucleotides in length, with interrupted palindromic sequences that can fold into hairpin structures that function as primers during initiation of DNA replication. The cap gene encodes the viral capsid protein, and the rep gene is involved in the replication and integration of AAV. AAV can infect a variety of cells, and the viral DNA can be integrated into human chromosome 19 in the presence of the rep product.

As used herein, the term “inverted terminal repeats” or “ITRs” as used herein refers to AAV viral cis-elements named so because of their symmetry. These elements are essential for efficient multiplication of an AAV genome. In the present invention, the term “ITR” refers to ITRs of known natural AAV serotypes, to chimeric ITRs formed by the fusion of ITR elements derived from different serotypes, and to functional variant thereof.

The production of a recombinant AAV particle may involve three plasmids, a plasmid comprising a polynucleotide construct for expressing an exogenous polynucleotide, a packaging plasmid encoding the REP and/or CAP proteins, and a helper plasmid.

As used herein, the term “polynucleotide construct” refers to a single-stranded or double-stranded polynucleotide, which is isolated from a naturally occurring gene or modified to contain a nucleic acid segment that does not naturally occur. When the polynucleotide construct contains the control sequences required to express the coding sequence of the present invention, the polynucleotide construct comprises an “expression cassette”.

As used herein, the term “polynucleotide” usually refers to generally a nucleic acid molecule (e.g., 100 bases and up to 30 kilobases in length) and a sequence that is either complementary (antisense) or identical (sense) to the sequence of a messenger RNA (mRNA) or miRNA fragment or molecule. The term can also refer to DNA or RNA molecules that are either transcribed or non-transcribed.

The term “exogenous polynucleotide” as used herein refers to a nucleotide sequence that does not originate from the host in which it is placed. It may be identical to the host's DNA or heterologous. An example is a sequence of interest inserted into a vector. Such exogenous DNA sequences may be derived from a variety of sources including DNA, cDNA, synthetic DNA, and RNA. Exogenous polynucleotides also encompass DNA sequences that encode antisense oligonucleotides.

As used herein, the term “expression cassette” refers to a polynucleotide segment comprising a polynucleotide encoding a polypeptide operably linked to additional nucleotides provided for the expression of the polynucleotide, for example, control sequence.

As used herein, the term “expression” includes any step involved in the production of a polypeptide, including but not limited to transcription, post-transcriptional modification, translation, post-translational modification, and secretion.

A “control sequence” includes all elements necessary or beneficial for the expression of the polynucleotide encoding the polypeptide of the present invention. Each control sequence may be natural or foreign to the nucleotide sequence encoding the polypeptide, or natural or foreign to each other. Such control sequences include, but are not limited to, leader sequence, polyadenylation sequence, propeptide sequence, promoter, enhancer, signal peptide sequence, and transcription terminator. At a minimum, control sequences include a promoter and signals for the termination of transcription and translation.

For example, the control sequence may be a suitable promoter sequence, a nucleotide sequence recognized by the host cell to express the polynucleotide encoding the polypeptide of the present invention. The promoter sequence contains a transcription control sequence that mediates the expression of the polypeptide. The promoter may be any nucleotide sequence that exhibits transcriptional activity in the selected host cell, for example, lac operon of E. coli. The promoters also include mutant, truncated and hybrid promoters, and can be obtained from genes encoding extracellular or intracellular polypeptides, which are homologous or heterologous to the host cell.

As used herein, the term “operably linked” herein refers to a configuration in which a control sequence is placed at an appropriate position relative to the coding sequence of the polynucleotide sequence, whereby the control sequence directs the expression of the polypeptide coding sequence.

The polynucleotide encoding the Nb24 can be subjected to various manipulations to improve the expression of the polypeptide. Before the insertion thereof into a vector, manipulation of the polynucleotide according to the expression vector or the host, such as codon optimization, is desirable or necessary. Techniques for modifying polynucleotide sequences with recombinant DNA methods are well known in the art.

The term “recombinant” as used herein refers to nucleic acids, vectors, polypeptides, or proteins that have been generated using DNA recombination (cloning) methods and are distinguishable from native or wild-type nucleic acids, vectors, polypeptides, or proteins.

The terms “polypeptide” and “protein” are used interchangeably herein and refer to a polymer of amino acids and includes full-length proteins and fragments thereof.

As used herein, the term “host cell” refers to, for example microorganisms, yeast cells, insect cells, and mammalian cells, that can be, or have been, used as recipients of rAAV vectors. The term includes the progeny of the original cell which has been transduced. Thus, a “host cell” as used herein generally refers to a cell which has been transduced with an exogenous DNA sequence. It is understood that the progeny of a single parental cell may not necessarily be completely identical in morphology or in genomic or total DNA complement to the original parent, due to natural, accidental, or deliberate mutation.

The term “pharmaceutically acceptable” as used herein refers to molecular entities and compositions that are physiologically tolerable and do not typically produce toxicity or an allergic or similar untoward reaction, such as gastric upset, dizziness and the like, when administered to a human.

The term “subject” as used herein includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, the term “Diabetic macular edema” (DME) refers to a disease with the accumulation of excess fluid in the extracellular space within the retina in the macular area, typically in the inner nuclear, outer plexiform, Henle's fiber layer, and subretinal space. Macular edema is a major cause of visual deterioration in diabetic retinopathy. It involves a wide spectrum of pathologic changes, including diffuse or focal edema and cystoid macular edema, which may have hard exudate. At present, anti-VEGF agents are the first-line treatment for DME requiring treatment. Since 2005, intravitreal bevacizumab has been used off-label for ocular conditions. FDA approved ranibizumab for DME in 2012, Aflibercept in 2014 and Brolucizumab and Faricimab in 2022.

### 2. The Construct for Expressing the Humanized Nanobody Against VEGF

Gene therapy aims to correct defective genes that underlie the development of diseases, and to introduce exogenous gene into the cell of interest in a subject to express the product of the exogenous gene that is useful for treating a certain disease. A common approach for this purpose involves the delivery of a functional gene to the nucleus. This gene may then be inserted into the genome of the cell of interest or may remain episomal. Delivery of a functional gene to a subject's target cells can be carried out via numerous methods, including the use of viral vectors. Among the many viral vectors available (e.g, retrovirus, lentivirus, adenovirus, and the like), AAV is gaining popularity as a versatile vector in gene therapy.

Vectors derived from AAV are particularly attractive for delivering genetic material because (i) they are able to infect (transduce) a wide variety of non-dividing and dividing cell types including muscle fibers and neurons; (ii) they are devoid of the virus structural genes, thereby eliminating the natural host cell responses to virus infection, e.g., interferon-mediated responses; (iii) wild-type viruses have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of the therapeutic transgenes (provided their gene products are not rejected). AAV vectors can also be produced at high titer and it has been reported that intra-arterial, intra-venous, or intra-peritoneal injections allow gene transfer to significant muscle regions in rodents through a single injection.

It has been reported that Nb24 is effective for blocking the binding of VEGF to VEGFR, thereby preventing or treating a disease associated with VEGF. It is desired to provide a polynucleotide construct for the long-term expression of Nb24 at a high level in a target cell.

The present invention thus intends to provide a polynucleotide construct, preferably an AAV vector, for expressing Nb24 in a cell of interest in a subject.

The present invention provides a polynucleotide construct comprising a first expression cassette comprising a first nucleotide sequence encoding a signal peptide and a Nb24 operably linked to a first promoter, wherein the first nucleotide sequence is codon optimized for the expression in a host, preferably in human.

In some embodiments, the first nucleotide sequence is selected from SEQ ID NOs: 1, 2, 3, 4, 5, 6, 7, 8, 9, 10 and 11. In some embodiments, the first nucleotide sequence is selected from SEQ ID NOs: 4, 5 and 7. In some embodiments, the first nucleotide sequence is SEQ ID NO: 5.

The inventors surprisingly found that the construct encoding bi-valent Nb24 achieved a higher expression than the mono-valent ones. Therefore, the present invention further provides a polynucleotide construct for expressing a bi-valent Nb24, i.e., two copies of Nb24 in a single polypeptide.

In some embodiments, the first expression cassette comprises a second nucleotide sequence linked to the first nucleotide sequence via a linker, preferably to the 3′ end of the first nucleotide sequence. In some embodiments, the second nucleotide sequence is selected from SEQ ID NO: 12, 13, 14, 15, 16, 17, 18, 19, 20, 21 and 22. In some embodiments, the second nucleotide sequence is selected from ID NOs: 15, 16 and 18.

In some embodiments, the first nucleotide sequence is SEQ ID NO: 5, and the second nucleotide sequence is SEQ ID NO: 15. In some embodiments, the first nucleotide sequence is SEQ ID NO: 5, and the second nucleotide sequence is SEQ ID NO: 16. In some embodiments, the first nucleotide sequence is SEQ ID NO: 5, and the second nucleotide sequence is SEQ ID NO: 18. In some embodiments, the first nucleotide sequence is SEQ ID NO: 4, and the second nucleotide sequence is SEQ ID NO: 15. In some embodiments, the first nucleotide sequence is SEQ ID NO: 4, and the second nucleotide sequence is SEQ ID NO: 16. In some embodiments, the first nucleotide sequence is SEQ ID NO: 4, and the second nucleotide sequence is SEQ ID NO: 18. In some embodiments, the first nucleotide sequence is SEQ ID NO: 7, and the second nucleotide sequence is SEQ ID NO: 15. In some embodiments, the first nucleotide sequence is SEQ ID NO: 7, and the second nucleotide sequence is SEQ ID NO: 16. In some embodiments, the first nucleotide sequence is SEQ ID NO: 7, and the second nucleotide sequence is SEQ ID NO: 18.

The inventors surprisingly found that the construct encoding bi-valent Nb24 with identical coding sequences resulted in poor virus quality (genome integrity). Therefore, in some embodiments, the first nucleotide sequence, over nucleotides 66-441 thereof, is not identical to the second nucleotide sequence.

A peptide linker can be generally short peptides with about 4-20 or more amino acids, such as combinations of Ser and Gly residues. In the construct of the present invention, the linker is a nucleotide linker encoding such a peptide linker. In some embodiments, the linker of the present invention encodes a peptide linker (G4S)n, n=1-4. In some embodiments the linker is selected from SEQ ID NOs: 36, 37, 38 and 39, or the degenerated variants thereof.

The construct will generally be transferred to mammalian cells (such as human cells) for expression. Such constructs often include promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control.

In some embodiments, the first promoter is a chicken beta-actin promoter or a promoter from CMV. In some embodiments, the first promoter comprises a nucleotide sequence of SEQ ID NO: 26 or 27, or a nucleotide sequence at least 70%, 75%, 80%, 85%, 90%, 95%, 96%, 97%, 98%, or 99% identical to SEQ ID NO: 26 or 27.

In some embodiments, the first expression cassette comprises a first enhancer. Preferably, the first enhancer is upstream of the first promoter. In some embodiments, the first enhancer is a cytomegalovirus (CMV) early enhancer. In some embodiments, the first enhancer comprises SEQ ID NO: 25, or a nucleotide sequence at least 70%, 75%, 80%, 85%, 90%, 95%, 96%, 97%, 98%, or 99% identical to SEQ ID NO: 25.

It is also desirable to include an intron derived from a eukaryotic organism in the construct to increase the expression in eukaryotic cells. In some embodiments, the first expression cassette comprises a first intron. Preferably, the first intron is upstream of the first nucleotide sequence.

The inventor surprisingly found that the incorporation of a longer intron led to an increased expression. In some embodiments, the first intron is at least 200 nucleotides in length. In some embodiments, the first intron comprises SEQ ID NO: 28, or a nucleotide sequence at least 70%, 75%, 80%, 85%, 90%, 95%, 96%, 97%, 98%, or 99% identical to SEQ ID NO: 28.

The construct further comprises polyadenylation signal sequence for the processing of the transcript. In some embodiments, the first expression cassette comprises a first polyadenylation signal sequence downstream of the coding sequence. In some embodiments, the first expression cassette polyadenylation signal sequence is selected from SEQ ID NOs: 31 and 32, or a nucleotide sequence at least 70%, 75%, 80%, 85%, 90%, 95%, 96%, 97%, 98%, or 99% identical to SEQ ID NO: 31 or 32.

The inventor further noted that a dual-cassette construct achieved a higher expression than the mono-cassette ones. Therefore, in some embodiments, the polynucleotide construct further comprises a second expression cassette comprising a third nucleotide sequence selected from SEQ ID NOs: 1, 2, 3, 4, 5, 6, 7, 8, 9, 10 and 11 operably linked to a second promoter. In some embodiments, the third nucleotide sequence is selected from SEQ ID NOs: 4, 5 and 7. In some embodiments, the third nucleotide sequence is SEQ ID NOs: 5.

In some embodiments, the second promoter is a chicken beta-actin promoter or a promoter from CMV. In some embodiments, the second promoter comprises a nucleotide sequence of SEQ ID NO: 26 or 27, or a nucleotide sequence at least 70%, 75%, 80%, 85%, 90%, 95%, 96%, 97%, 98%, or 99% identical to SEQ ID NO: 26 or 27.

In some embodiments, the second expression cassette comprises a second enhancer, preferably upstream of the second promoter. In some embodiments, the second enhancer is a CMV early enhancer. In some embodiments, the second enhancer comprises SEQ ID NO: 25, or a nucleotide sequence at least 70%, 75%, 80%, 85%, 90%, 95%, 96%, 97%, 98%, or 99% identical to SEQ ID NO: 25.

In some embodiments, the second expression cassette comprises a second polyadenylation signal sequence downstream of the coding sequence. In some embodiments, the second polyadenylation signal sequence is selected from SEQ ID NOs: 31 and 32, or a nucleotide sequence at least 70%, 75%, 80%, 85%, 90%, 95%, 96%, 97%, 98%, or 99% identical to SEQ ID NO: 31 or 32.

In some embodiments, the second expression cassette comprises a second intron, preferably upstream of the third nucleotide sequence. In some embodiments, the second intron is at least 200 nucleotides in length. In some embodiments, the second intron comprises SEQ ID NO: 28, or a nucleotide sequence at least 70%, 75%, 80%, 85%, 90%, 95%, 96%, 97%, 98%, or 99% identical to SEQ ID NO: 28.

In some embodiments, the construct comprises a single expression cassette comprising, in the order of 5′ to 3′, an enhancer, a promoter, an intron, a nucleotide sequence encoding Nb24 and a polyadenylation signal sequence.

In a specific embodiment, the expression cassette comprises, in the order of 5′ to 3′, SEQ ID NO: 25, SEQ ID NO: 26, SEQ ID NO: 28, SEQ ID NO: 1, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 2, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 3, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 6, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 8, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 9, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 10, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 11, SEQ ID NO: 31.

In a specific embodiment, the expression cassette comprises, in the order of 5′ to 3′, SEQ ID NO: 25, SEQ ID NO: 27, SEQ ID NO: 28, SEQ ID NO: 1, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 2, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 3, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 6, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 8, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 9, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 10, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 11, SEQ ID NO: 31.

In a specific embodiment, the expression cassette comprises, in the order of 5′ to 3′, SEQ ID NO: 25, SEQ ID NO: 26, SEQ ID NO: 28, SEQ ID NO: 1, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 2, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 3, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 6, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 8, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 9, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 10, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 11, SEQ ID NO: 32.

In a specific embodiment, the expression cassette comprises, in the order of 5′ to 3′, SEQ ID NO: 25, SEQ ID NO: 27, SEQ ID NO: 28, SEQ ID NO: 1, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 2, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 3, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 6, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 8, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 9, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 10, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 11, SEQ ID NO: 32.

In some embodiments, the construct comprises a single expression cassette comprising, in the order of 5′ to 3′, an enhancer, a promoter, an intron, a coding sequence comprising a first nucleotide sequence, a linker and a second nucleotide sequence, and a polyadenylation signal sequence.

In a specific embodiment, the expression cassette comprises, in the order of 5′ to 3′, SEQ ID to 3′, SEQ ID NO: 25, SEQ ID NO: 26, SEQ ID NO: 28, SEQ ID NO: 5, SEQ ID NO: 37, SEQ ID NO: 16, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 38, SEQ ID NO: 16, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 39, SEQ ID NO: 16, SEQ ID NO: 31.

In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 36, SEQ ID NO: 15, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 37, SEQ ID NO: 15, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 38, SEQ ID NO: 15, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 39, SEQ ID NO: 15, SEQ ID NO: 31.

In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 36, SEQ ID NO: 18, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 37, SEQ ID NO: 18, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 38, SEQ ID NO: 18, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 39, SEQ ID NO: 18, SEQ ID NO: 31.

In a specific embodiment, the expression cassette comprises, in the order of 5′ to 3′, SEQ ID to 3′, SEQ ID NO: 25, SEQ ID NO: 26, SEQ ID NO: 28, SEQ ID NO: 4, SEQ ID NO: 37, SEQ ID NO: 15, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 38, SEQ ID NO: 15, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 39, SEQ ID NO: 15, SEQ ID NO: 31.

In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 36, SEQ ID NO: 16, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 37, SEQ ID NO: 16, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 38, SEQ ID NO: 16, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 39, SEQ ID NO: 16, SEQ ID NO: 31.

In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 36, SEQ ID NO: 18, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 37, SEQ ID NO: 18, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 38, SEQ ID NO: 18, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 39, SEQ ID NO: 18, SEQ ID NO: 31.

In a specific embodiment, the expression cassette comprises, in the order of 5′ to 3′, SEQ ID to 3′, SEQ ID NO: 25, SEQ ID NO: 26, SEQ ID NO: 28, SEQ ID NO: 7, SEQ ID NO: 37, SEQ ID NO: 18, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 38, SEQ ID NO: 18, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 39, SEQ ID NO: 18, SEQ ID NO: 31.

In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 36, SEQ ID NO: 15, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 37, SEQ ID NO: 15, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 38, SEQ ID NO: 15, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 39, SEQ ID NO: 15, SEQ ID NO: 31.

In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 36, SEQ ID NO: 16, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 37, SEQ ID NO: 16, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 38, SEQ ID NO: 16, SEQ ID NO: 31. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 39, SEQ ID NO: 16, SEQ ID NO: 31.

In a specific embodiment, the expression cassette comprises, in the order of 5′ to 3′, SEQ ID to 3′, SEQ ID NO: 25, SEQ ID NO: 27, SEQ ID NO: 28, SEQ ID NO: 5, SEQ ID NO: 37, SEQ ID NO: 16, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 38, SEQ ID NO: 16, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 39, SEQ ID NO: 16, SEQ ID NO: 31.

In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 36, SEQ ID NO: 15, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 37, SEQ ID NO: 15, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 38, SEQ ID NO: 15, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 39, SEQ ID NO: 15, SEQ ID NO: 31.

In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 36, SEQ ID NO: 18, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 37, SEQ ID NO: 18, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 38, SEQ ID NO: 18, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 39, SEQ ID NO: 18, SEQ ID NO: 31.

In a specific embodiment, the expression cassette comprises, in the order of 5′ to 3′, SEQ ID to 3′, SEQ ID NO: 25, SEQ ID NO: 27, SEQ ID NO: 28, SEQ ID NO: 4, SEQ ID NO: 37, SEQ ID NO: 15, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 38, SEQ ID NO: 15, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 39, SEQ ID NO: 15, SEQ ID NO: 31.

In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 36, SEQ ID NO: 16, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 37, SEQ ID NO: 16, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 38, SEQ ID NO: 16, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 39, SEQ ID NO: 16, SEQ ID NO: 31.

In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 36, SEQ ID NO: 18, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 37, SEQ ID NO: 18, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 38, SEQ ID NO: 18, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 39, SEQ ID NO: 18, SEQ ID NO: 31.

In a specific embodiment, the expression cassette comprises, in the order of 5′ to 3′, SEQ ID to 3′, SEQ ID NO: 25, SEQ ID NO: 27, SEQ ID NO: 28, SEQ ID NO: 7, SEQ ID NO: 37, SEQ ID NO: 18, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 38, SEQ ID NO: 18, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 39, SEQ ID NO: 18, SEQ ID NO: 31.

In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 36, SEQ ID NO: 15, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 37, SEQ ID NO: 15, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 38, SEQ ID NO: 15, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 39, SEQ ID NO: 15, SEQ ID NO: 31.

In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 36, SEQ ID NO: 16, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 37, SEQ ID NO: 16, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 38, SEQ ID NO: 16, SEQ ID NO: 31. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 39, SEQ ID NO: 16, SEQ ID NO: 31.

In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 36, SEQ ID NO: 16, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 37, SEQ ID NO: 16, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 38, SEQ ID NO: 16, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 39, SEQ ID NO: 16, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 36, SEQ ID NO: 15, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 37, SEQ ID NO: 15, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 38, SEQ ID NO: 15, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 39, SEQ ID NO: 15, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 36, SEQ ID NO: 18, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 37, SEQ ID NO: 18, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 38, SEQ ID NO: 18, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 39, SEQ ID NO: 18, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 36, SEQ ID NO: 15, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 37, SEQ ID NO: 15, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 38, SEQ ID NO: 15, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 39, SEQ ID NO: 15, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 36, SEQ ID NO: 16, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 37, SEQ ID NO: 16, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 38, SEQ ID NO: 16, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 39, SEQ ID NO: 16, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 36, SEQ ID NO: 18, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 37, SEQ ID NO: 18, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 38, SEQ ID NO: 18, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 39, SEQ ID NO: 18, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 36, SEQ ID NO: 18, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 37, SEQ ID NO: 18, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 38, SEQ ID NO: 18, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 39, SEQ ID NO: 18, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 36, SEQ ID NO: 15, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 37, SEQ ID NO: 15, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 38, SEQ ID NO: 15, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 39, SEQ ID NO: 15, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 36, SEQ ID NO: 16, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 37, SEQ ID NO: 16, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 38, SEQ ID NO: 16, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 39, SEQ ID NO: 16, SEQ ID NO: 32.

In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 36, SEQ ID NO: 16, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 37, SEQ ID NO: 16, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 38, SEQ ID NO: 16, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 39, SEQ ID NO: 16, SEQ ID NO: 32.

In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 36, SEQ ID NO: 15, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 37, SEQ ID NO: 15, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 38, SEQ ID NO: 15, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 39, SEQ ID NO: 15, SEQ ID NO: 32.

In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 36, SEQ ID NO: 18, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 37, SEQ ID NO: 18, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 38, SEQ ID NO: 18, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 5, SEQ ID NO: 39, SEQ ID NO: 18, SEQ ID NO: 32.

In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 36, SEQ ID NO: 15, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 37, SEQ ID NO: 15, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 38, SEQ ID NO: 15, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 39, SEQ ID NO: 15, SEQ ID NO: 32.

In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 36, SEQ ID NO: 16, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 37, SEQ ID NO: 16, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 38, SEQ ID NO: 16, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 39, SEQ ID NO: 16, SEQ ID NO: 32.

In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 36, SEQ ID NO: 18, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 37, SEQ ID NO: 18, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 38, SEQ ID NO: 18, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 4, SEQ ID NO: 39, SEQ ID NO: 18, SEQ ID NO: 32.

In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 36, SEQ ID NO: 18, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 37, SEQ ID NO: 18, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 38, SEQ ID NO: 18, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 39, SEQ ID NO: 18, SEQ ID NO: 32.

In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 36, SEQ ID NO: 15, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 37, SEQ ID NO: 15, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 38, SEQ ID NO: 15, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 39, SEQ ID NO: 15, SEQ ID NO: 32.

In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 36, SEQ ID NO: 16, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 37, SEQ ID NO: 16, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 38, SEQ ID NO: 16, SEQ ID NO: 32. In a specific embodiment, the expression cassette comprises, in the order of 5′ to 3′, SEQ ID NO: 25, SEQ ID NO: 27, SEQ ID NO: 28, SEQ ID NO: 7, SEQ ID NO: 39, SEQ ID NO: 16, SEQ ID NO: 32.

In some embodiments, the construct comprises a first expression cassette and a second expression cassette.

In a specific embodiment, the first expression cassette comprises, in the order of 5′ to 3′, SEQ ID NO: 25, SEQ ID NO: 26, SEQ ID NO: 28, SEQ ID NO: 1, SEQ ID NO: 31. In a specific embodiment, the first expression cassette comprises, in the order of 5′ to 3′, SEQ ID NO: 25, SEQ ID NO: 26, SEQ ID NO: 28, SEQ ID NO: 2, SEQ ID NO: 31. In a specific embodiment, the first expression cassette comprises, in the order of 5′ to 3′, SEQ ID NO: 25, SEQ ID NO: 26, SEQ ID NO: 28, SEQ ID NO: 3, SEQ ID NO: 31. In a specific embodiment, the first expression cassette comprises, in the order of 5′ to 3′, SEQ ID NO: 25, SEQ ID NO: 26, SEQ ID NO: 28, SEQ ID NO: 4, SEQ ID NO: 31. In a specific embodiment, the first expression cassette comprises, in the order of 5′ to 3′, SEQ ID NO: 25, SEQ ID NO: 26, SEQ ID NO: 28, SEQ ID NO: 5, SEQ ID NO: 31. In a specific embodiment, the first expression cassette comprises, in the order of 5′ to 3′, SEQ ID NO: 25, SEQ ID NO: 26, SEQ ID NO: 28, SEQ ID NO: 6, SEQ ID NO: 31. In a specific embodiment, the first expression cassette comprises, in the order of 5′ to 3′, SEQ ID NO: 25, SEQ ID NO: 26, SEQ ID NO: 28, SEQ ID NO: 7, SEQ ID NO: 31. In a specific embodiment, the first expression cassette comprises, in the order of 5′ to 3′, SEQ ID NO: 25, SEQ ID NO: 26, SEQ ID NO: 28, SEQ ID NO: 8, SEQ ID NO: 31. In a specific embodiment, the first expression cassette comprises, in the order of 5′ to 3′, SEQ ID NO: 25, SEQ ID NO: 26, SEQ ID NO: 28, SEQ ID NO: 9, SEQ ID NO: 31. In a specific embodiment, the first expression cassette comprises, in the order of 5′ to 3′, SEQ ID NO: 25, SEQ ID NO: 26, SEQ ID NO: 28, SEQ ID NO: 10, SEQ ID NO: 31. In a specific embodiment, the first expression cassette comprises, in the order of 5′ to 3′, SEQ ID NO: 25, SEQ ID NO: 26, SEQ ID NO: 28, SEQ ID NO: 11, SEQ ID NO: 31.

In a specific embodiment, the first expression cassette comprises, in the order of 5′ to 3′, SEQ ID NO: 25, SEQ ID NO: 27, SEQ ID NO: 28, SEQ ID NO: 1, SEQ ID NO: 31. In a specific embodiment, the first expression cassette comprises, in the order of 5′ to 3′, SEQ ID NO: 25, SEQ ID NO: 27, SEQ ID NO: 28, SEQ ID NO: 2, SEQ ID NO: 31. In a specific embodiment, the first expression cassette comprises, in the order of 5′ to 3′, SEQ ID NO: 25, SEQ ID NO: 27, SEQ ID NO: 28, SEQ ID NO: 3, SEQ ID NO: 31. In a specific embodiment, the first expression cassette comprises, in the order of 5′ to 3′, SEQ ID NO: 25, SEQ ID NO: 27, SEQ ID NO: 28, SEQ ID NO: 4, SEQ ID NO: 31. In a specific embodiment, the first expression cassette comprises, in the order of 5′ to 3′, SEQ ID NO: 25, SEQ ID NO: 27, SEQ ID NO: 28, SEQ ID NO: 5, SEQ ID NO: 31. In a specific embodiment, the first expression cassette comprises, in the order of 5′ to 3′, SEQ ID NO: 25, SEQ ID NO: 27, SEQ ID NO: 28, SEQ ID NO: 6, SEQ ID NO: 31. In a specific embodiment, the first expression cassette comprises, in the order of 5′ to 3′, SEQ ID NO: 25, SEQ ID NO: 27, SEQ ID NO: 28, SEQ ID NO: 7, SEQ ID NO: 31. In a specific embodiment, the first expression cassette comprises, in the order of 5′ to 3′, SEQ ID NO: 25, SEQ ID NO: 27, SEQ ID NO: 28, SEQ ID NO: 8, SEQ ID NO: 31. In a specific embodiment, the first expression cassette comprises, in the order of 5′ to 3′, SEQ ID NO: 25, SEQ ID NO: 27, SEQ ID NO: 28, SEQ ID NO: 9, SEQ ID NO: 31. In a specific embodiment, the first expression cassette comprises, in the order of 5′ to 3′, SEQ ID NO: 25, SEQ ID NO: 27, SEQ ID NO: 28, SEQ ID NO: 10, SEQ ID NO: 31. In a specific embodiment, the first expression cassette comprises, in the order of 5′ to 3′, SEQ ID NO: 25, SEQ ID NO: 27, SEQ ID NO: 28, SEQ ID NO: 11, SEQ ID NO: 31.

In a specific embodiment, the first expression cassette comprises, in the order of 5′ to 3′, SEQ ID NO: 25, SEQ ID NO: 26, SEQ ID NO: 28, SEQ ID NO: 1, SEQ ID NO: 32. In a specific embodiment, the first expression cassette comprises, in the order of 5′ to 3′, SEQ ID NO: 25, SEQ ID NO: 26, SEQ ID NO: 28, SEQ ID NO: 2, SEQ ID NO: 32. In a specific embodiment, the first expression cassette comprises, in the order of 5′ to 3′, SEQ ID NO: 25, SEQ ID NO: 26, SEQ ID NO: 28, SEQ ID NO: 3, SEQ ID NO: 32. In a specific embodiment, the first expression cassette comprises, in the order of 5′ to 3′, SEQ ID NO: 25, SEQ ID NO: 26, SEQ ID NO: 28, SEQ ID NO: 4, SEQ ID NO: 32. In a specific embodiment, the first expression cassette comprises, in the order of 5′ to 3′, SEQ ID NO: 25, SEQ ID NO: 26, SEQ ID NO: 28, SEQ ID NO: 5, SEQ ID NO: 32. In a specific embodiment, the first expression cassette comprises, in the order of 5′ to 3′, SEQ ID NO: 25, SEQ ID NO: 26, SEQ ID NO: 28, SEQ ID NO: 6, SEQ ID NO: 32. In a specific embodiment, the first expression cassette comprises, in the order of 5′ to 3′, SEQ ID NO: 25, SEQ ID NO: 26, SEQ ID NO: 28, SEQ ID NO: 7, SEQ ID NO: 32. In a specific embodiment, the first expression cassette comprises, in the order of 5′ to 3′, SEQ ID NO: 25, SEQ ID NO: 26, SEQ ID NO: 28, SEQ ID NO: 8, SEQ ID NO: 32. In a specific embodiment, the first expression cassette comprises, in the order of 5′ to 3′, SEQ ID NO: 25, SEQ ID NO: 26, SEQ ID NO: 28, SEQ ID NO: 9, SEQ ID NO: 32. In a specific embodiment, the first expression cassette comprises, in the order of 5′ to 3′, SEQ ID NO: 25, SEQ ID NO: 26, SEQ ID NO: 28, SEQ ID NO: 10, SEQ ID NO: 32. In a specific embodiment, the first expression cassette comprises, in the order of 5′ to 3′, SEQ ID NO: 25, SEQ ID NO: 26, SEQ ID NO: 28, SEQ ID NO: 11, SEQ ID NO: 32.

In a specific embodiment, the first expression cassette comprises, in the order of 5′ to 3′, SEQ ID NO: 25, SEQ ID NO: 27, SEQ ID NO: 28, SEQ ID NO: 1, SEQ ID NO: 32. In a specific embodiment, the first expression cassette comprises, in the order of 5′ to 3′, SEQ ID NO: 25, SEQ ID NO: 27, SEQ ID NO: 28, SEQ ID NO: 2, SEQ ID NO: 32. In a specific embodiment, the first expression cassette comprises, in the order of 5′ to 3′, SEQ ID NO: 25, SEQ ID NO: 27, SEQ ID NO: 28, SEQ ID NO: 3, SEQ ID NO: 32. In a specific embodiment, the first expression cassette comprises, in the order of 5′ to 3′, SEQ ID NO: 25, SEQ ID NO: 27, SEQ ID NO: 28, SEQ ID NO: 4, SEQ ID NO: 32. In a specific embodiment, the first expression cassette comprises, in the order of 5′ to 3′, SEQ ID NO: 25, SEQ ID NO: 27, SEQ ID NO: 28, SEQ ID NO: 5, SEQ ID NO: 32. In a specific embodiment, the first expression cassette comprises, in the order of 5′ to 3′, SEQ ID NO: 25, SEQ ID NO: 27, SEQ ID NO: 28, SEQ ID NO: 6, SEQ ID NO: 32. In a specific embodiment, the first expression cassette comprises, in the order of 5′ to 3′, SEQ ID NO: 25, SEQ ID NO: 27, SEQ ID NO: 28, SEQ ID NO: 7, SEQ ID NO: 32. In a specific embodiment, the first expression cassette comprises, in the order of 5′ to 3′, SEQ ID NO: 25, SEQ ID NO: 27, SEQ ID NO: 28, SEQ ID NO: 8, SEQ ID NO: 32. In a specific embodiment, the first expression cassette comprises, in the order of 5′ to 3′, SEQ ID NO: 25, SEQ ID NO: 27, SEQ ID NO: 28, SEQ ID NO: 9, SEQ ID NO: 32. In a specific embodiment, the first expression cassette comprises, in the order of 5′ to 3′, SEQ ID NO: 25, SEQ ID NO: 27, SEQ ID NO: 28, SEQ ID NO: 10, SEQ ID NO: 32. In a specific embodiment, the first expression cassette comprises, in the order of 5′ to 3′, SEQ ID NO: 25, SEQ ID NO: 27, SEQ ID NO: 28, SEQ ID NO: 11, SEQ ID NO: 32.

In a specific embodiment, the second expression cassette comprises, in the order of 5′ to 3′, SEQ ID NO: 25, SEQ ID NO: 26, SEQ ID NO: 28, SEQ ID NO: 1, SEQ ID NO: 31. In a specific embodiment, the second expression cassette comprises, in the order of 5′ to 3′, SEQ ID NO: 25, SEQ ID NO: 26, SEQ ID NO: 28, SEQ ID NO: 2, SEQ ID NO: 31. In a specific embodiment, the second expression cassette comprises, in the order of 5′ to 3′, SEQ ID NO: 25, SEQ ID NO: 26, SEQ ID NO: 28, SEQ ID NO: 3, SEQ ID NO: 31. In a specific embodiment, the second expression cassette comprises, in the order of 5′ to 3′, SEQ ID NO: 25, SEQ ID NO: 26, SEQ ID NO: 28, SEQ ID NO: 4, SEQ ID NO: 31. In a specific embodiment, the second expression cassette comprises, in the order of 5′ to 3′, SEQ ID NO: 25, SEQ ID NO: 26, SEQ ID NO: 28, SEQ ID NO: 5, SEQ ID NO: 31. In a specific embodiment, the second expression cassette comprises, in the order of 5′ to 3′, SEQ ID NO: 25, SEQ ID NO: 26, SEQ ID NO: 28, SEQ ID NO: 6, SEQ ID NO: 31. In a specific embodiment, the second expression cassette comprises, in the order of 5′ to 3′, SEQ ID NO: 25, SEQ ID NO: 26, SEQ ID NO: 28, SEQ ID NO: 7, SEQ ID NO: 31. In a specific embodiment, the second expression cassette comprises, in the order of 5′ to 3′, SEQ ID NO: 25, SEQ ID NO: 26, SEQ ID NO: 28, SEQ ID NO: 8, SEQ ID NO: 31. In a specific embodiment, the second expression cassette comprises, in the order of 5′ to 3′, SEQ ID NO: 25, SEQ ID NO: 26, SEQ ID NO: 28, SEQ ID NO: 9, SEQ ID NO: 31. In a specific embodiment, the second expression cassette comprises, in the order of 5′ to 3′, SEQ ID NO: 25, SEQ ID NO: 26, SEQ ID NO: 28, SEQ ID NO: 10, SEQ ID NO: 31. In a specific embodiment, the second expression cassette comprises, in the order of 5′ to 3′, SEQ ID NO: 25, SEQ ID NO: 26, SEQ ID NO: 28, SEQ ID NO: 11, SEQ ID NO: 31.

In a specific embodiment, the second expression cassette comprises, in the order of 5′ to 3′, SEQ ID NO: 25, SEQ ID NO: 27, SEQ ID NO: 28, SEQ ID NO: 1, SEQ ID NO: 31. In a specific embodiment, the second expression cassette comprises, in the order of 5′ to 3′, SEQ ID NO: 25, SEQ ID NO: 27, SEQ ID NO: 28, SEQ ID NO: 2, SEQ ID NO: 31. In a specific embodiment, the second expression cassette comprises, in the order of 5′ to 3′, SEQ ID NO: 25, SEQ ID NO: 27, SEQ ID NO: 28, SEQ ID NO: 3, SEQ ID NO: 31. In a specific embodiment, the second expression cassette comprises, in the order of 5′ to 3′, SEQ ID NO: 25, SEQ ID NO: 27, SEQ ID NO: 28, SEQ ID NO: 4, SEQ ID NO: 31. In a specific embodiment, the second expression cassette comprises, in the order of 5′ to 3′, SEQ ID NO: 25, SEQ ID NO: 27, SEQ ID NO: 28, SEQ ID NO: 5, SEQ ID NO: 31. In a specific embodiment, the second expression cassette comprises, in the order of 5′ to 3′, SEQ ID NO: 25, SEQ ID NO: 27, SEQ ID NO: 28, SEQ ID NO: 6, SEQ ID NO: 31. In a specific embodiment, the second expression cassette comprises, in the order of 5′ to 3′, SEQ ID NO: 25, SEQ ID NO: 27, SEQ ID NO: 28, SEQ ID NO: 7, SEQ ID NO: 31. In a specific embodiment, the second expression cassette comprises, in the order of 5′ to 3′, SEQ ID NO: 25, SEQ ID NO: 27, SEQ ID NO: 28, SEQ ID NO: 8, SEQ ID NO: 31. In a specific embodiment, the second expression cassette comprises, in the order of 5′ to 3′, SEQ ID NO: 25, SEQ ID NO: 27, SEQ ID NO: 28, SEQ ID NO: 9, SEQ ID NO: 31. In a specific embodiment, the second expression cassette comprises, in the order of 5′ to 3′, SEQ ID NO: 25, SEQ ID NO: 27, SEQ ID NO: 28, SEQ ID NO: 10, SEQ ID NO: 31. In a specific embodiment, the second expression cassette comprises, in the order of 5′ to 3′, SEQ ID NO: 25, SEQ ID NO: 27, SEQ ID NO: 28, SEQ ID NO: 11, SEQ ID NO: 31.

In a specific embodiment, the second expression cassette comprises, in the order of 5′ to 3′, SEQ ID NO: 25, SEQ ID NO: 26, SEQ ID NO: 28, SEQ ID NO: 1, SEQ ID NO: 32. In a specific embodiment, the second expression cassette comprises, in the order of 5′ to 3′, SEQ ID NO: 25, SEQ ID NO: 26, SEQ ID NO: 28, SEQ ID NO: 2, SEQ ID NO: 32. In a specific embodiment, the second expression cassette comprises, in the order of 5′ to 3′, SEQ ID NO: 25, SEQ ID NO: 26, SEQ ID NO: 28, SEQ ID NO: 3, SEQ ID NO: 32. In a specific embodiment, the second expression cassette comprises, in the order of 5′ to 3′, SEQ ID NO: 25, SEQ ID NO: 26, SEQ ID NO: 28, SEQ ID NO: 4, SEQ ID NO: 32. In a specific embodiment, the second expression cassette comprises, in the order of 5′ to 3′, SEQ ID NO: 25, SEQ ID NO: 26, SEQ ID NO: 28, SEQ ID NO: 5, SEQ ID NO: 32. In a specific embodiment, the second expression cassette comprises, in the order of 5′ to 3′, SEQ ID NO: 25, SEQ ID NO: 26, SEQ ID NO: 28, SEQ ID NO: 6, SEQ ID NO: 32. In a specific embodiment, the second expression cassette comprises, in the order of 5′ to 3′, SEQ ID NO: 25, SEQ ID NO: 26, SEQ ID NO: 28, SEQ ID NO: 7, SEQ ID NO: 32. In a specific embodiment, the second expression cassette comprises, in the order of 5′ to 3′, SEQ ID NO: 25, SEQ ID NO: 26, SEQ ID NO: 28, SEQ ID NO: 8, SEQ ID NO: 32. In a specific embodiment, the second expression cassette comprises, in the order of 5′ to 3′, SEQ ID NO: 25, SEQ ID NO: 26, SEQ ID NO: 28, SEQ ID NO: 9, SEQ ID NO: 32. In a specific embodiment, the second expression cassette comprises, in the order of 5′ to 3′, SEQ ID NO: 25, SEQ ID NO: 26, SEQ ID NO: 28, SEQ ID NO: 10, SEQ ID NO: 32. In a specific embodiment, the second expression cassette comprises, in the order of 5′ to 3′, SEQ ID NO: 25, SEQ ID NO: 26, SEQ ID NO: 28, SEQ ID NO: 11, SEQ ID NO: 32.

In a specific embodiment, the second expression cassette comprises, in the order of 5′ to 3′, SEQ ID NO: 25, SEQ ID NO: 27, SEQ ID NO: 28, SEQ ID NO: 1, SEQ ID NO: 32. In a specific embodiment, the second expression cassette comprises, in the order of 5′ to 3′, SEQ ID NO: 25, SEQ ID NO: 27, SEQ ID NO: 28, SEQ ID NO: 2, SEQ ID NO: 32. In a specific embodiment, the second expression cassette comprises, in the order of 5′ to 3′, SEQ ID NO: 25, SEQ ID NO: 27, SEQ ID NO: 28, SEQ ID NO: 3, SEQ ID NO: 32. In a specific embodiment, the second expression cassette comprises, in the order of 5′ to 3′, SEQ ID NO: 25, SEQ ID NO: 27, SEQ ID NO: 28, SEQ ID NO: 4, SEQ ID NO: 32. In a specific embodiment, the second expression cassette comprises, in the order of 5′ to 3′, SEQ ID NO: 25, SEQ ID NO: 27, SEQ ID NO: 28, SEQ ID NO: 5, SEQ ID NO: 32. In a specific embodiment, the second expression cassette comprises, in the order of 5′ to 3′, SEQ ID NO: 25, SEQ ID NO: 27, SEQ ID NO: 28, SEQ ID NO: 6, SEQ ID NO: 32. In a specific embodiment, the second expression cassette comprises, in the order of 5′ to 3′, SEQ ID NO: 25, SEQ ID NO: 27, SEQ ID NO: 28, SEQ ID NO: 7, SEQ ID NO: 32. In a specific embodiment, the second expression cassette comprises, in the order of 5′ to 3′, SEQ ID NO: 25, SEQ ID NO: 27, SEQ ID NO: 28, SEQ ID NO: 8, SEQ ID NO: 32. In a specific embodiment, the second expression cassette comprises, in the order of 5′ to 3′, SEQ ID NO: 25, SEQ ID NO: 27, SEQ ID NO: 28, SEQ ID NO: 9, SEQ ID NO: 32. In a specific embodiment, the second expression cassette comprises, in the order of 5′ to 3′, SEQ ID NO: 25, SEQ ID NO: 27, SEQ ID NO: 28, SEQ ID NO: 10, SEQ ID NO: 32. In a specific embodiment, the second expression cassette comprises, in the order of 5′ to 3′, SEQ ID NO: 25, SEQ ID NO: 27, SEQ ID NO: 28, SEQ ID NO: 11, SEQ ID NO: 32.

In some embodiments, the construct comprises the genome of a recombinant AAV. In some embodiments, the construct comprises 5′ and 3′ inverted terminal repeats (ITRs) of adeno-associated virus (AAV). In some embodiments, the 5′ and 3′ ITRs are AAV ITR130 and/or AAV ITR 105. In some embodiments, both the 5′ and 3′ ITRs are AAV ITR 130. In some embodiments, the 5′ ITR is AAV ITR105, and the 3′ ITR is AAV ITR130. In some embodiments, 5′ ITR130 is SEQ ID NO: 33. In some embodiments, 3′ ITR 130 is SEQ ID NO: 34. In some embodiment, the 5′ ITR105 is SEQ ID NO: 35.

### 3. Recombinant AAVs and Pharmaceutical Composition

The present invention further provides a recombinant AAV comprising a genome comprising the polynucleotide construct of the invention.

In some embodiments, the polynucleotide construct comprises a first expression cassette comprising a first nucleotide sequence encoding a signal peptide and a Nb24 operably linked to a first promoter, wherein the first nucleotide sequence is codon optimized for the expression in a host, preferably in human.

In some embodiments, the first nucleotide sequence is selected from SEQ ID NOs: 1, 2, 3, 4, 5, 6, 7, 8, 9, 10 and 11. In some embodiments, the first nucleotide sequence is selected from SEQ ID NOs: 4, 5 and 7. In some embodiments, the first nucleotide sequence is SEQ ID NO: 5.

The invertors surprisingly found that the construct encoding bi-valent Nb24 achieved a higher expression than the mono-valent ones. Therefore, the present invention further provides a polynucleotide construct for expressing a bi-valent Nb24, i.e., two copies of Nb24 in a single polypeptide.

In some embodiments, the first expression cassette comprises a second nucleotide sequence linked to the first nucleotide sequence via a linker, preferably to the 3′ end of the first nucleotide sequence. In some embodiments, the second nucleotide sequence is selected from SEQ ID NO: 12, 13, 14, 15, 16, 17, 18, 19, 20, 21 and 22. In some embodiments, the second nucleotide sequence is selected from ID NOs: 15, 16 and 18.

The invertors surprisingly found that the construct encoding bi-valent Nb24 with identical coding sequences resulted in poor virus quality (genome integrity). Therefore, in some embodiments, the first nucleotide sequence, over nucleotides 66-441 thereof, is not identical to the second nucleotide sequence.

In some embodiments, the first nucleotide sequence is SEQ ID NO: 5, and the second nucleotide sequence is SEQ ID NO: 15. In some embodiments, the first nucleotide sequence is SEQ ID NO: 5, and the second nucleotide sequence is SEQ ID NO: 18. In some embodiments, the first nucleotide sequence is SEQ ID NO: 4, and the second nucleotide sequence is SEQ ID NO: 16. In some embodiments, the first nucleotide sequence is SEQ ID NO: 4, and the second nucleotide sequence is SEQ ID NO: 18. In some embodiments, the first nucleotide sequence is SEQ ID NO: 7, and the second nucleotide sequence is SEQ ID NO: 15. In some embodiments, the first nucleotide sequence is SEQ ID NO: 7, and the second nucleotide sequence is SEQ ID NO: 16.

A peptide linker can be generally short peptides with about 4-20 or more amino acids, such as combinations of Ser and Gly residues. In the construct of the present invention, the linker is a nucleotide linker encoding such a peptide linker. In some embodiments, the linker of the present invention encodes a peptide linker (G4S)n, n=1-4. In some embodiments the linker is selected from SEQ ID NOs: 36, 37, 38 and 39, or the degenerated variants thereof.

In some embodiments, the polynucleotide construct comprises a coding sequence for bi-valent Nb24 comprising, in the order of 5′ to 3′,
SEQ ID NO: 5, SEQ ID NO: 37, and SEQ ID NO: 15,
SEQ ID NO: 5, SEQ ID NO: 37, and SEQ ID NO: 18,
SEQ ID NO: 4, SEQ ID NO: 37, and SEQ ID NO: 16,
SEQ ID NO: 4, SEQ ID NO: 37, and SEQ ID NO: 18,
SEQ ID NO: 7, SEQ ID NO: 37, and SEQ ID NO: 15,
SEQ ID NO: 7, SEQ ID NO: 37, and SEQ ID NO: 16,
SEQ ID NO: 5, SEQ ID NO: 38, and SEQ ID NO: 15,
SEQ ID NO: 5, SEQ ID NO: 38, and SEQ ID NO: 18,
SEQ ID NO: 4, SEQ ID NO: 38, and SEQ ID NO: 16,
SEQ ID NO: 4, SEQ ID NO: 38, and SEQ ID NO: 18,
SEQ ID NO: 7, SEQ ID NO: 38, and SEQ ID NO: 15, or
SEQ ID NO: 7, SEQ ID NO: 38, and SEQ ID NO: 16.

In some embodiments, the first promoter is a chicken beta-actin promoter or a promoter from CMV. In some embodiments, the first promoter comprises a nucleotide sequence of SEQ ID NO: 26 or 27, or a nucleotide sequence at least 70%, 75%, 80%, 85%, 90%, 95%, 96%, 97%, 98%, or 99% identical to SEQ ID NO: 26 or 27.

In some embodiments, the first expression cassette comprises a first enhancer. Preferably, the first enhancer is upstream of the first promoter. In some embodiments, the first enhancer is a cytomegalovirus (CMV) early enhancer. In some embodiments, the first enhancer comprises SEQ ID NO: 25, or a nucleotide sequence at least 70%, 75%, 80%, 85%, 90%, 95%, 96%, 97%, 98%, or 99% identical to SEQ ID NO: 25.

It is also desirable to include an intron derived from a eukaryotic organism in the construct to increase the expression in eukaryotic cells. In some embodiments, the first expression cassette comprises a first intron. Preferably, the first intron is upstream of the first nucleotide sequence.

The inventor surprisingly found that the incorporation of a longer intron led to an increased expression. In some embodiments, the first intron is at least 200 nucleotides in length. In some embodiments, the first intron comprises SEQ ID NO: 28, or a nucleotide sequence at least 70%, 75%, 80%, 85%, 90%, 95%, 96%, 97%, 98%, or 99% identical to SEQ ID NO: 28.

The construct further comprises polyadenylation signal sequence for the processing of the transcript. In some embodiments, the first expression cassette comprises a first polyadenylation signal sequence downstream of the coding sequence. In some embodiments, the first expression cassette polyadenylation signal sequence is selected from SEQ ID NOs: 31 and 32, or a nucleotide sequence at least 70%, 75%, 80%, 85%, 90%, 95%, 96%, 97%, 98%, or 99% identical to SEQ ID NO: 31 or 32.

In some embodiments, the construct comprises a single expression cassette comprising, in the order of 5′ to 3′, an enhancer, a promoter, an intron, a coding sequence comprising a first nucleotide sequence, a linker and a second nucleotide sequence, and a polyadenylation signal sequence.

In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 37, SEQ ID NO: 15, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 38, SEQ ID NO: 15, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 37, SEQ ID NO: 18, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 5, SEQ ID NO: 38, SEQ ID NO: 18, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 37, SEQ ID NO: 16, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 38, SEQ ID NO: 16, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 37, SEQ ID NO: 18, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 4, SEQ ID NO: 38, SEQ ID NO: 18, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 37, SEQ ID NO: 15, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 38, SEQ ID NO: 15, SEQ ID NO: 32.

In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 37, SEQ ID NO: 16, SEQ ID NO: 32. In a specific embodiment, the expression cassette comprises, in the order of 5′ to 3′, SEQ ID NO: 25, SEQ ID NO: 26, SEQ ID NO: 28, SEQ ID NO: 7, SEQ ID NO: 38, SEQ ID NO: 16, SEQ ID NO: 32.

In some embodiments, the construct comprises 5′ and 3′ inverted terminal repeats (ITRs) of adeno-associated virus (AAV). In some embodiments, the 5′ and 3′ ITRs are AAV ITR130 and/or AAV ITR 105. In some embodiments, both the 5′ and 3′ ITRs are AAV ITR130. In some embodiments, the 5′ ITR is AAV ITR 105, and the 3′ ITR is AAV ITR 130. In some embodiments, 5′ ITR 130 is SEQ ID NO: 33. In some embodiments, 3′ ITR130 is SEQ ID NO: 34. In some embodiment, the 5′ ITR105 is SEQ ID NO: 35.

The present invention also provides a method for preparing the rAAV. In some embodiments, the rAAV is prepared by a system containing a transgene plasmid comprising the genome of the rAAV, a packaging plasmid encoding the REP and/or CAP proteins, and a helper plasmid, e.g., a host cell such as a mammalian cell comprising the transgene plasmid comprising the genome of the rAAV, the packaging plasmid encoding the REP and/or CAP proteins, and the helper plasmid. Therefore, the present invention also provides a vector such as a plasmid comprising the genome of the rAAV of the invention.

In some embodiments, the rAAV can be packaged as described in Crosson S M et al. (Helper-free Production of Laboratory Grade AAV and Purification by Iodixanol Density Gradient Centrifugation. Mol Ther Methods Clin Dev. 2018; 10:1-7).

In some embodiments, the rAAV of the invention can be selected from human serotype 1 AAV (hAAV1), hAAV 2, hAAV 3, hAAV 4, hAAV5, hAAV6, hAAV7, hAAV8, hAAV9, hAAV10, and hAAV11. In some embodiments, the rAAV is hAAV2. In some embodiments, the rAAV is hAAV9.

Pharmaceutical compositions containing the AAV of the invention can be formulated in any conventional manner by mixing a selected amount of the rAAV with one or more pharmaceutically acceptable carriers or excipients.

Selection of the carrier or excipient is within the skill of the administering professional and can depend upon a number of parameters. These include, for example, the mode of administration (i.e., systemic, oral, local, topical or any other mode) and the disease to be treated. Pharmaceutical carriers or vehicles suitable for administration of the rAAV provided herein include any such carriers known to those skilled in the art to be suitable for the particular mode of administration. In some embodiments, the disease to be treated is Wet AMD. In some embodiments, the pharmaceutical composition is formulated for intravitreal injection.

### 4. Treatment of VEGF-Associated Diseases

The present invention provides a method of preventing or treating a disease associated with VEGF, such as the overexpression of VEGF, comprising administering the rAAV or the pharmaceutical composition of the present invention to a subject in need thereof. In some embodiments, the disease is Wet-AMD or Diabetic macular edema (DME). In some embodiments, the rAAV, or the pharmaceutical composition is administered by intravitreal injection.

The present invention further provides use of the polynucleotide construct, the rAAV or the pharmaceutical composition of the present invention in the preparation of a medicament for preventing or treating a disease associated with VEGF, such as the overexpression of VEGF. In some embodiments, the disease is Wet-AMD or DME. In some embodiments, the medicament is administered by intravitreal injection.

The present invention further provides the rAAV or the pharmaceutical composition of the present invention, for use of preventing or treating a disease associated with VEGF, such as the overexpression of VEGF. In some embodiments, the disease is Wet-AMD or DME. In some embodiments, the medicament is administered by intravitreal injection.

### Embodiments

Clause 1. A polynucleotide construct comprising a first expression cassette comprising a first nucleotide sequence selected from SEQ ID NOs: 1, 2, 3, 4, 5, 6, 7, 8, 9, 10 and 11, preferably selected from SEQ ID NOs: 4, 5 and 7, operably linked to a first promoter.

Clause 2. The polynucleotide construct of clause 1, wherein the first expression cassette comprises a second nucleotide sequence linked to the first nucleotide sequence via a linker, preferably wherein the first nucleotide sequence, over nucleotides 66-441 thereof, is not identical to the second nucleotide sequence.

Clause 3. The polynucleotide construct of clause 1 or 2, wherein the second nucleotide sequence is selected from SEQ ID NOs: 12, 13, 14, 15, 16, 17, 18, 19, 20, 21 and 22, preferably selected from SEQ ID NOs: 15, 16 and 18.

Clause 4. The polynucleotide construct of clause 2 or 3, wherein the linker is a nucleotide sequence selected from SEQ ID NOs: 36, 37, 38 and 39, preferably selected from SEQ ID NOs: 37 and 38.

Clause 5. The polynucleotide construct of any of clauses 2-4, wherein the polynucleotide construct comprises a coding sequence for bi-valent Nb24 comprising, in the order of 5′ to 3′,
SEQ ID NO: 5, SEQ ID NO: 37, and SEQ ID NO: 15,
SEQ ID NO: 5, SEQ ID NO: 37, and SEQ ID NO: 18,
SEQ ID NO: 4, SEQ ID NO: 37, and SEQ ID NO: 16,
SEQ ID NO: 4, SEQ ID NO: 37, and SEQ ID NO: 18,
SEQ ID NO: 7, SEQ ID NO: 37, and SEQ ID NO: 15,
SEQ ID NO: 7, SEQ ID NO: 37, and SEQ ID NO: 16,
SEQ ID NO: 5, SEQ ID NO: 38, and SEQ ID NO: 15,
SEQ ID NO: 5, SEQ ID NO: 38, and SEQ ID NO: 18,
SEQ ID NO: 4, SEQ ID NO: 38, and SEQ ID NO: 16,
SEQ ID NO: 4, SEQ ID NO: 38, and SEQ ID NO: 18,
SEQ ID NO: 7, SEQ ID NO: 38, and SEQ ID NO: 15, or
SEQ ID NO: 7, SEQ ID NO: 38, and SEQ ID NO: 16.

Clause 6. The polynucleotide construct of any of clauses 1-5, wherein the first promoter is a chicken beta-actin promoter or a promoter from CMV.

Clause 7. The polynucleotide construct of clause 6, wherein the first promoter comprises a nucleotide sequence of SEQ ID NO: 26 or 27.

Clause 8. The polynucleotide construct of any of clauses 1-7, wherein the first expression cassette comprises a first enhancer.

Clause 9. The polynucleotide construct of clause 8, wherein the first enhancer is upstream of the first promoter.

Clause 10. The polynucleotide construct of clause 8, wherein the first enhancer is a cytomegalovirus (CMV) early enhancer.

Clause 11. The polynucleotide construct of any of clauses 7-10, wherein the first enhancer comprises SEQ ID NO: 25.

Clause 12. The polynucleotide construct of any of clauses 1-11, wherein the first expression cassette comprises a first polyadenylation signal sequence downstream of the coding sequence.

Clause 13. The polynucleotide construct of clause 12, wherein the first polyadenylation signal sequence is selected from SEQ ID NOs: 31 and 32.

Clause 14. The polynucleotide construct of any of clauses 1-13, wherein the first expression cassette comprises a first intron, preferably upstream of the first nucleotide sequence.

Clause 15. The polynucleotide construct of clause 14, wherein the first intron is at least 200 nucleotides in length.

Clause 16. The polynucleotide construct of clause 14, wherein the first intron comprises SEQ ID NO: 28.

Clause 17. The polynucleotide construct of clause 1 or 2, further comprising a second expression cassette comprising a third nucleotide sequence selected from SEQ ID NOs: 1, 2, 3, 4, 5, 6, 7, 8, 9, 10 and 11 operably linked to a second promoter.

Clause 18. The polynucleotide construct of clause 17, wherein the third nucleotide sequence selected from SEQ ID NOs: 4, 5 and 7.

Clause 19. The polynucleotide construct of clause 17 or 18, wherein the second promoter is a chicken beta-actin promoter or a promoter from CMV.

Clause 20. The polynucleotide construct of clause 19, wherein the second promoter comprises SEQ ID NO: 26 or 27.

Clause 21. The polynucleotide construct of any of clauses 17-20, wherein the second expression cassette comprises a second enhancer.

Clause 22. The polynucleotide construct of clause 21, wherein the second enhancer is upstream of the second promoter.

Clause 23. The polynucleotide construct of clause 21, wherein the second enhancer is a CMV early enhancer.

Clause 24. The polynucleotide construct of clause 23, wherein the second enhancer comprises SEQ ID NO: 25.

Clause 25. The polynucleotide construct of any of clauses 17-24, wherein the second expression cassette comprises a second polyadenylation signal sequence downstream of the coding sequence.

Clause 26. The polynucleotide construct of clause 25, wherein the second polyadenylation signal sequence is selected from SEQ ID NOs: 31 and 32.

Clause 27. The polynucleotide construct of any of clauses 17-26, wherein the second expression cassette comprises a second intron, preferably upstream of the third nucleotide sequence.

Clause 28. The polynucleotide construct of clause 27, wherein the second intron is at least 200 nucleotides in length.

Clause 29. The polynucleotide construct of clause 27, wherein the second intron comprises SEQ ID NO: 28.

Clause 30. The polynucleotide construct of any of clause 1-29, wherein the construct comprises the genome of a recombinant AAV.

Clause 31. The polynucleotide construct of clause 30, wherein the construct comprises 5′ and 3′ inverted terminal repeat (ITR) sequences derived from adeno-associated virus (AAV).

Clause 32. The polynucleotide construct of clause 31, wherein the 5′ and 3′ ITRs are AAV ITR130 and/or AAV ITR 105.

Clause 33. The polynucleotide construct of clause 32, wherein both the 5′ and 3′ ITRs are AAV ITR130.

Clause 34. A recombinant adeno-associated virus (rAAV) comprising a genome comprising the polynucleotide construct of any of clauses 1-33.

Clause 35. A pharmaceutical composition comprising the AAV of clause 34.

Clause 36. A method of preventing or treating a disease associated with VEGF, comprising administering the rAAV of clause 34 or the pharmaceutical composition of clause 35 to a subject in need thereof.

Clause 37. The method of clause 36, wherein the disease is Wet-AMD or Diabetic macular edema (DME).

Clause 38. The method of clause 37, wherein the rAAV or the pharmaceutical composition is administered by intravitreal injection.

Clause 39. A host cell comprising the construct of any of clauses 1-33, or the rAAV of clause 34.

Clause 40. Use of the polynucleotide construct of any of clauses 1-33, the rAAV of clause 34, the pharmaceutical composition of clause 35, or the host cell of clause 39 in the preparation of a medicament for preventing or treating a disease associated with VEGF in a subject in need thereof.

Clause 41. The use of clause 40, wherein the disease is Wet-AMD or DME.

Clause 42. The use of clause 41, wherein the medicament is administered by intravitreal injection.

Clause 43. The rAAV of clause 34 or the pharmaceutical composition of clause 35, for use of preventing or treating a disease associated with VEGF, such as the overexpression of VEGF.

Clause 44. The rAAV or pharmaceutical composition for use of clause 43, wherein the disease is Wet-AMD or DME.

Clause 45. The rAAV or pharmaceutical composition for use of clause 44, wherein the rAAV or the pharmaceutical composition is administered by intravitreal injection.

### Benefits of the Invention

The present invention provides polynucleotides, constructs, vectors and rAAVs that increase the expression of Nb24, increase the activity of Nb24 (binding to VEGF and/or inhibition to VEGFR such as VEGFR2) and/or improve virus quality.

### EXAMPLES

The following Examples are provided for illustration only, rather than for any limitation to the present application.

### Example 1. Construction of Vectors Comprising Codon Optimized Sequence Encoding a VEGF Nanobody or a VEGF Antibody

### 1.1. Construction of Nb24 Nanobody (Humanized) Codon Optimized Plasmids

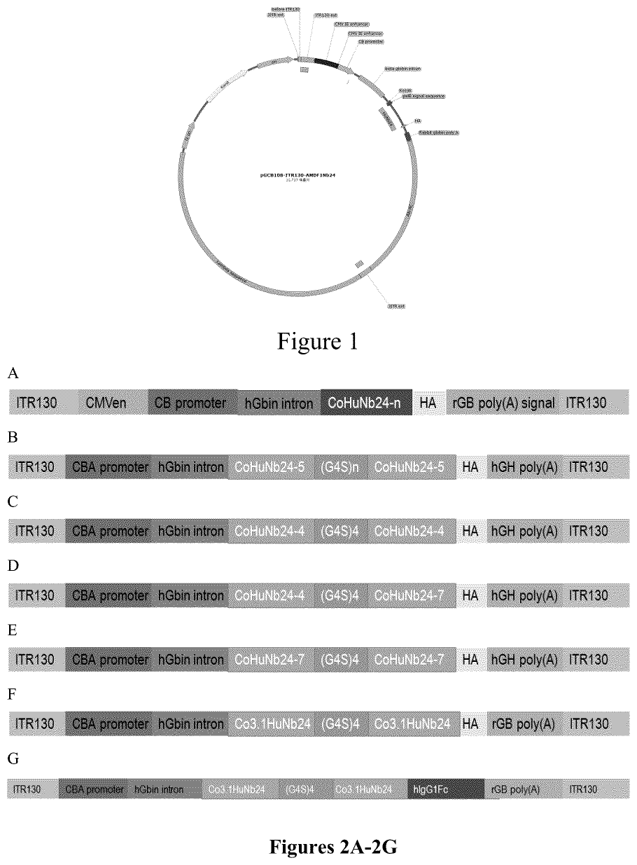

The pGCB108 plasmid along with the cytomegalovirus (CMV) early enhancer element (SEQ ID NO: 25) and the chicken beta-actin promoter (SEQ ID NO: 26) was Gibson Assembled with the first exon and the first intron of chicken beta-actin gene (referred to as F1, SEQ ID NO: 28) and a codon optimized sequence encoding Nb24 nanobody (selected from SEQ ID NOs: 1, 2, 3, 4, 5, 6, 7, 8, 9, 10 and 11, including signal sequence), and rGBpoly A signal sequence (SEQ ID NO: 31) with NEBuilder HiFi DNA Assembly Master Mix (NEB, Catalog E2621), resulting in 11 plasmids pGCB108-ITR130-AMDF1Nb24-n (n=1-11, see FIG. 1, and FIG. 2A). Restriction mapping (AgeI (R3552L) and KpnI (R3142L), both from NEB, according to the manufactory's instructions) and DNA sequencing (GENEWIZ, Suzhou, China) were carried out to confirm correct plasmid construction.

### 1.2. Construction of Bivalent Nb24 Nanobody Plasmids

The bivalent Nb24 nanobody plasmids were constructed by replacing the rGBpoly A signal sequence (SEQ ID NO: 31) of pGCB108-ITR130-AMDF1Nb24-5 with hGHpolyA sequence (SEQ ID NO: 32), resulting in the plasmid pGCB108-ITR130-AMDF1huNb24-5-hGHpA; and ligating two Nb24-5 Sequences (SEQ ID NO: 5 and SEQ ID NO: 16) with a linker (G4S)n by Gibson Assembly, resulting in the plasmids pGCB108-ITR130-AMDF1Nb24-5-(G4S)n-Nb24-5-HA (n=1-4, FIG. 2B) comprising SEQ ID NO: 5, G4S linker (SEQ ID NO: 36, 37, 38, or 39), and SEQ ID NO: 16, as well as plasmids pGCB108-ITR130-AMDF1Nb24-5-(G4S)2-Nb24-7-HA comprising SEQ ID NO: 5, SEQ ID NO: 38, and SEQ ID NO: 18, and pGCB108-ITR130-AMDF1Nb24-7-(G4S)2-Nb24-5-HA comprising SEQ ID NO: 7, SEQ ID NO: 38 and SEQ ID NO: 16.

A plasmid pGCB108-ITR130-AMDF1Nb24-5_stuffer was constructed by adding a stuffer sequence SEQ ID NO: 49 into pGCB108-ITR130-AMDF1huNb24-5-hGHpA down-streaming of SEQ ID NO: 32. A plasmid pGCB108-ITR130-AMDF1Nb24-5-(G4S)4-Nb24-5-HA stuffer was constructed by inserting nucleotides 1-1800 of SEQ ID NO: 49 into pGCB108-ITR130-AMDF1Nb24-5-(G4S)4-Nb24-5-HA downstream of the hGHpolyA sequence.

The following bivalent plasmids were constructed with a similar method based on plasmids pGCB108-ITR130-AMDF1Nb24-4 and pGCB108-ITR130-AMDF1Nb24-7, with pGCB108-ITR130-AMDF1huNb24-4-hGHpA and pGCB108-ITR130-AMDF1huNb24-7-hGHpA as the intermediates:
pGCB108-ITR130-AMDF1Nb24-4-(G4S)4-Nb24-4-HA (FIG. 2C) comprising SEQ ID NO: 4, SEQ ID NO: 36, and SEQ ID NO: 15,
pGCB108-ITR130-AMDF1Nb24-4-(G4S)4-Nb24-7-HA (FIG. 2D) comprising SEQ ID NO: 4, SEQ ID NO: 36, and SEQ ID NO: 18, and
pGCB108-ITR130-AMDF1Nb24-7-(G4S)4-Nb24-7-HA (FIG. 2E) comprising SEQ ID NO: 7, SEQ ID NO: 36, and SEQ ID NO: 15.

Additional bivalent plasmids were constructed by replacing the signal sequence (nucleotides 1-66 of SEQ ID NO: 5) in pGCB108-ITR130-AMDF1Nb24-5 with IL2 signal sequence (SEQ ID NO: 41); replacing the Nb24-5 sequence (SEQ ID NO: 16) with two Nb24 Sequences (Co3.1HuNb24, SEQ ID NO: 40) linked with a linker (G4S)4; and optionally replacing the HA tag (SEQ ID NO: 30) with hlgG1Fc (SEQ ID NO: 42), resulting in the following plasmids:
pGCB108-ITR130-AMDF1 Bi-huNb24-pCDNA3.1+-HA_IL2sig (FIG. 2F); and
pGCB108-ITR130-AMDF1 Bi-huNb24-hIgG1Fc (FIG. 2G).

Restriction mapping and DNA sequencing were carried out to confirm correct plasmid construction as described in Example 1.1.

### 1.3. Construction of a Benchmark Anti-VEGF Plasmid

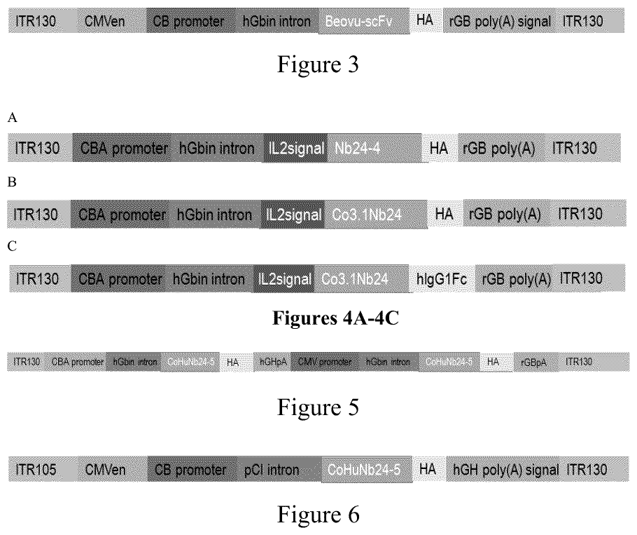

The pGCB108-ITR130-AMDF1-Beovu-scFv plasmid (FIG. 3) was constructed as follows. The pGCB108 plasmid along with the cytomegalovirus (CMV) early enhancer element and the chicken beta-actin promoter was Gibson Assembled with the first exon and the first intron of chicken beta-actin gene (F1), and a PCR product of Beovu-scFv sequence (SEQ ID NO: 43). Restriction mapping and DNA sequencing were carried out to confirm correct plasmid construction as described in Example 1.1.

### 1.4. Construction of IL2 Signal Peptide Nb24 Nanobody Plasmids

1) The plasmid pGCB108-ITR130-AMDF1Nb24-4(IL2 signal)-HA (FIG. 4A) was constructed by replacing the sequence encoding signal peptide of Nb24-4-HA (positions 1-66 of SEQ ID NO: 4) in plasmid pGCB108-ITR130-AMDF1Nb24-4 with the sequence encoding IL2 signal peptide (SEQ ID NO: 41) by Gibson Assembly.
2) The plasmid pGCB108-ITR130-AMDF1Co3.1Nb24(IL2 signal)-HA (FIG. 4B) was constructed by replacing the Nb24-4 sequence in pGCB108-ITR130-AMDF1Nb24-4(IL2 signal)-HA with Co3.1Nb24 (SEQ ID NO: 40) DNA sequences, and the plasmid pGCB108-ITR130-AMDF1Co3.1Nb24(IL2 signal)-hlgG1Fc (FIG. 4C) was constructed by further replacing the sequence encoding the HA tag with a sequence encoding hlgG1Fc (SEQ ID NO: 42). Restriction mapping and DNA sequencing were carried out to confirm correct plasmid construction as described in Example 1.1.

### 1.5. Construction of Dual Promoter Plasmid

The plasmid pGCB108-ITR130-AMDF1Nb24-5-HA-CMV-AMDF1-Nb24-5-HA (FIG. 5) was constructed as follows: the promoter of pGCB108-ITR130-AMDF1Nb24-5 was replaced by CMV promoter (SEQ ID NO: 44) using T4 DNA ligation; the GOI of CMV-F1Nb24-5-rGBA was amplified by PCR with additional Restriction Enzyme (NotI); and the dual promoter plasmid was in trans built by ligating the GOI of CMV-FINb24-5-rGBpA downstream of the hGHpolyA sequence in pGCB108-ITR130-AMDF1huNb24-5-hGHpA using restriction enzyme digestion and T4 DNA ligation. Restriction mapping and DNA sequencing were carried out to confirm correct plasmid construction as described in Example 1.1

1.6. Construction of Plasmid with Different Intron and ITR

The plasmid pCB108-5ITR130-CBA-pCI-Nb24-5-hGHpA (FIG. 6) was constructed by changing the F1 in the plasmid pGCB108-ITR130-AMDF1Nb24-5 to pCI (SEQ ID NO: 29), and replacing the 5ITR130 (SEQ ID NO: 33) with 5ITR105 (SEQ ID NO: 35) using T4 DNA ligation (T4 DNA ligase, NEB, M0202L) according to the manufactory's instructions. Restriction mapping and DNA sequencing were carried out to confirm correct plasmid construction as described in Example 1.1.

### Example 2. The Expression of Nb24 Nanobody with the Codon Optimized Plasmids

The Expi293 cells (Thermo Fisher Scientific) were cultured according to manufactory's protocol at 37° C., 8% CO₂, 80% humidity. The Expi293 cells were seeded in 12-well plates at the density of 2.5×10⁶ cells/well, and transfected with the plasmids pGCB108-ITR130-AMDF1Nb24-n (n=1-11) according to manufactory's protocol (Thermo Fisher Scientific). The plasmid pGCB108-ITR130-AMDF1-Beovu-scFv was added to each of the CoNb24 transgene with a ratio of 1:9 as the reference showing the transfection efficiency. The supernatants of the cultures were collected 72h after transfection.

Equal volume of the supernatants of Nb24 were loaded on a NuPAGE 4-12% Bis-Tris Gel for PAGE (1 μl reducing+9 μl 4×li-cor protein loading+30 μl sup, 200V, 22 min). The proteins were transferred onto a nitrocellulose membrane using iBlot® 2 Gel Transfer Device (Thermo Fisher Scientific) and iBlot® 2 Transfer Stack (nitrocellulose, regular size). After one hour incubation with a blocking buffer (Odyssey, Li-Cor), and rinse for 3 times (1 min/rinse), membranes were probed with primary antibody (Anti-HA rabbit (Thermo, PA1-985), 1:5000, 4° C., overnight) in the same buffer, followed by rinse for 3 times (1 min/rinse), and then, the incubation with Donkey Anti-rabbit 800CW, (1:5000) (catalog #REF926-32213, Li-Cor) for 2h at RT. After washing with TBST (diluted Pierce™ 20×TBS Tween™ 20 Buffer, Catalog number: 28360 of Thermo Fisher Scientific) for 2h (15 min/wash), immunoreaction was visualized using Odyssey Infrared Imager (Li-Cor).

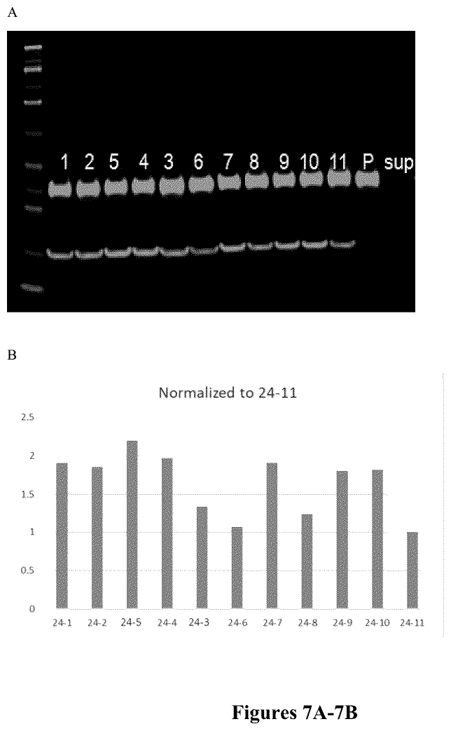

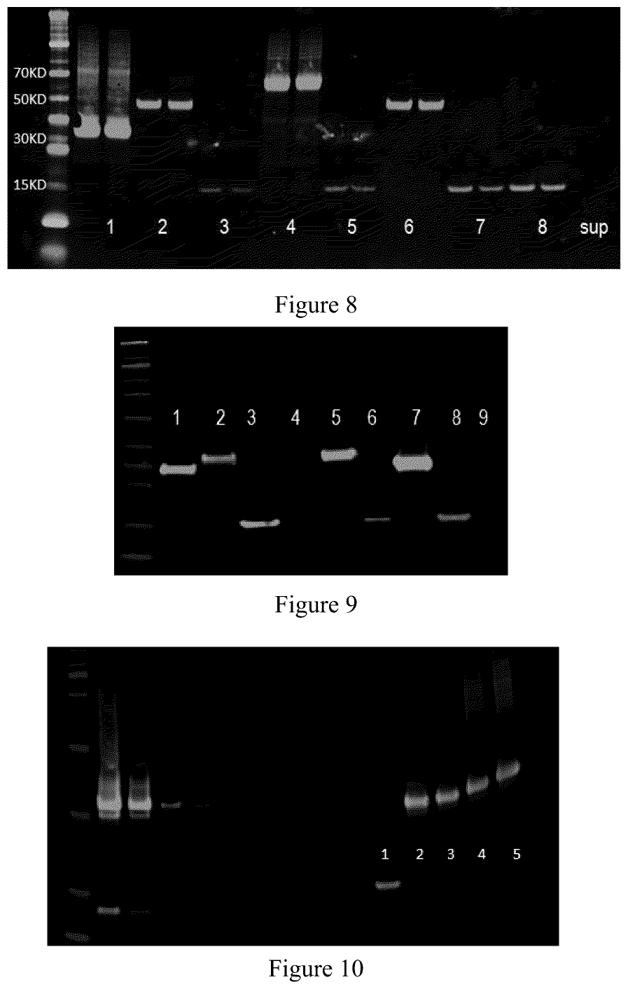

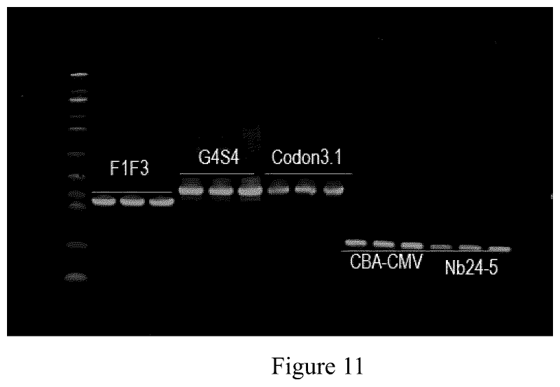

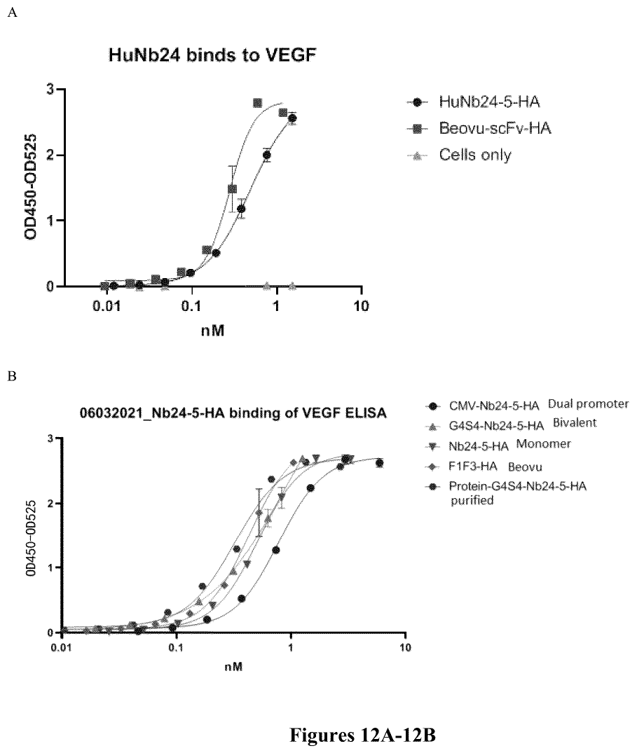

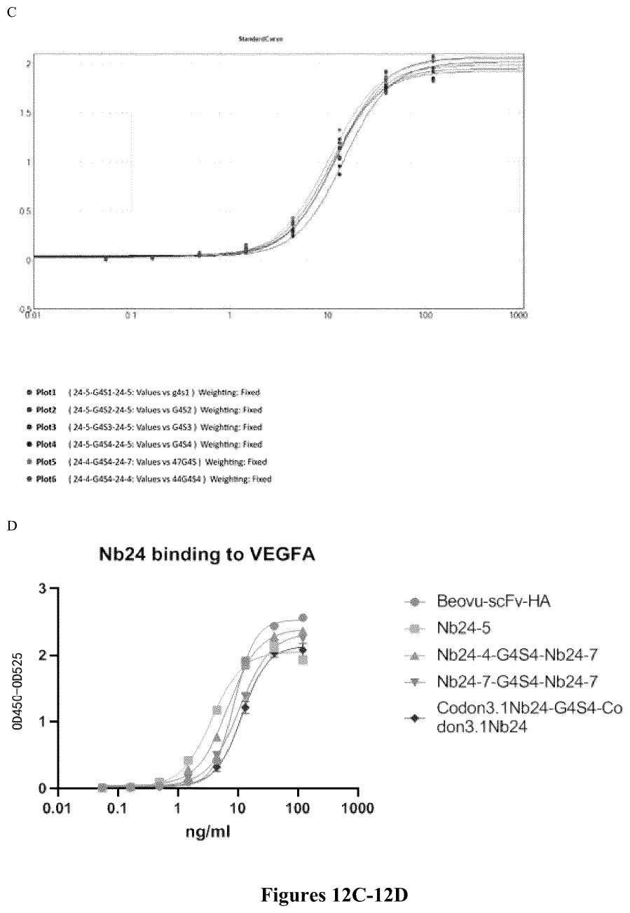

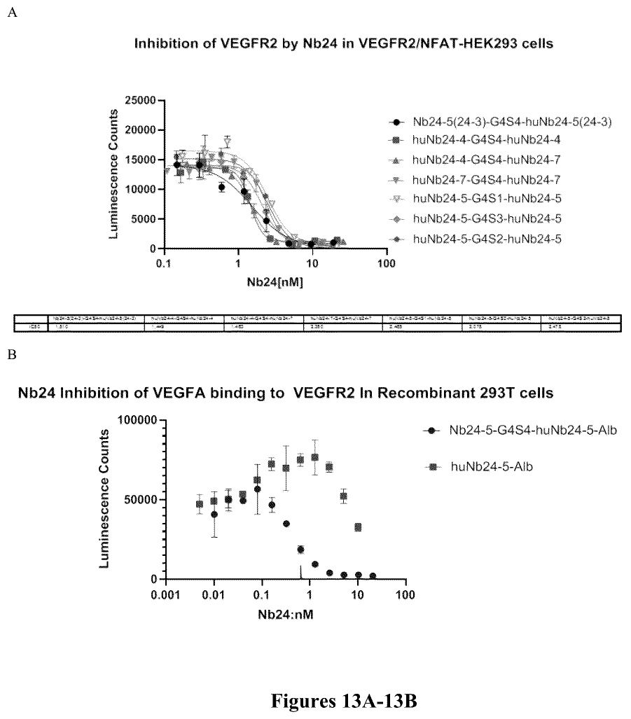

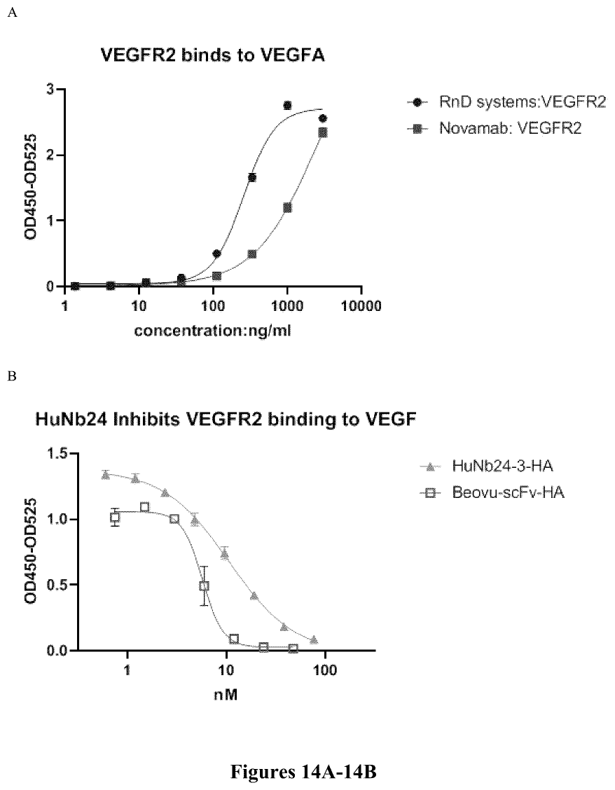

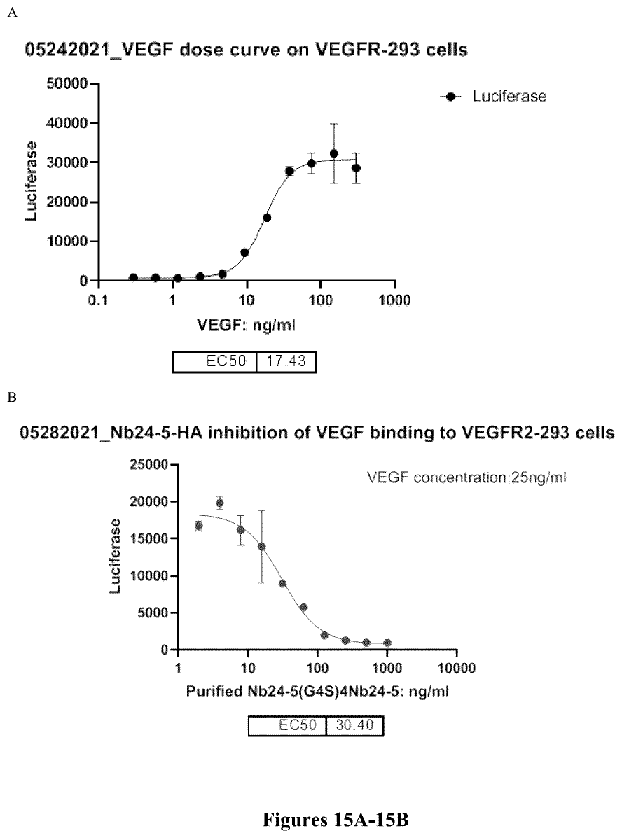

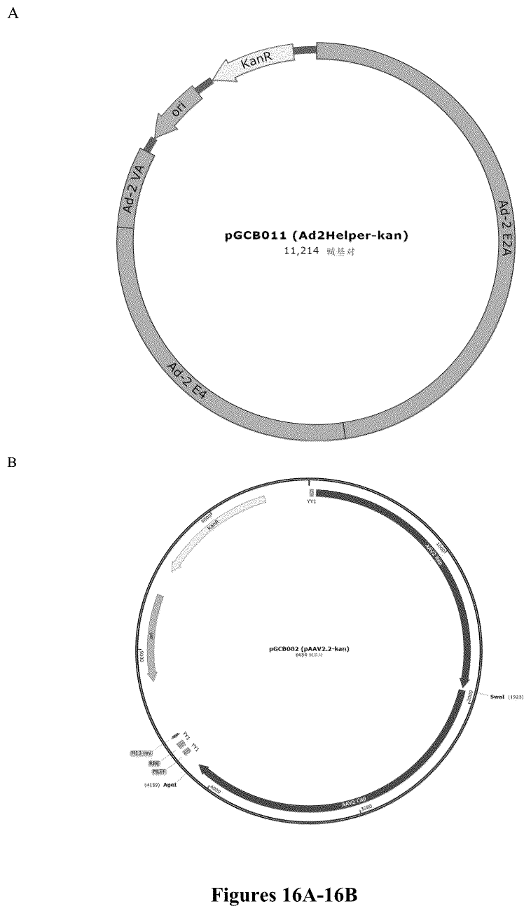

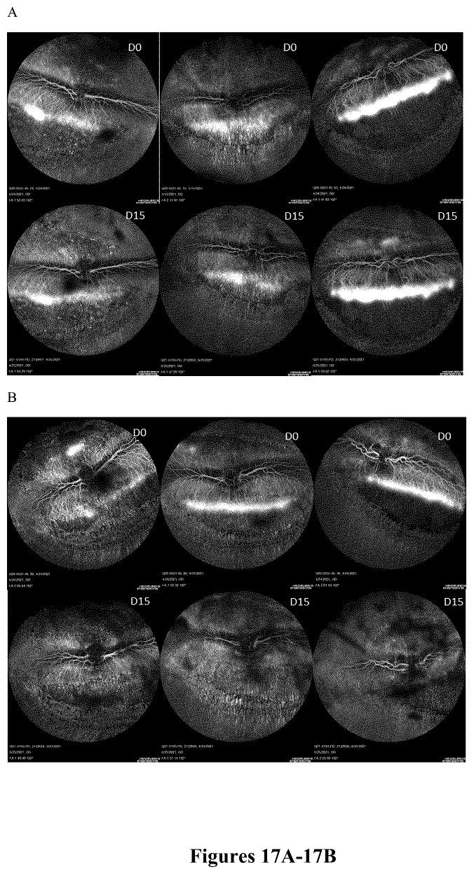

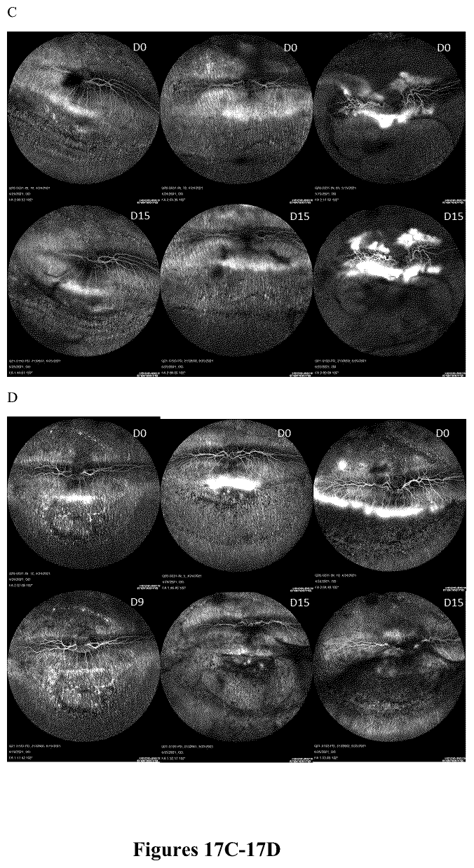

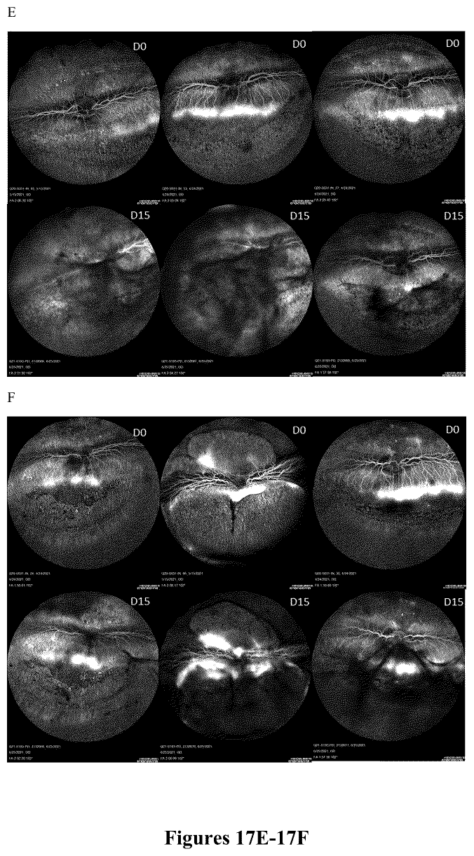

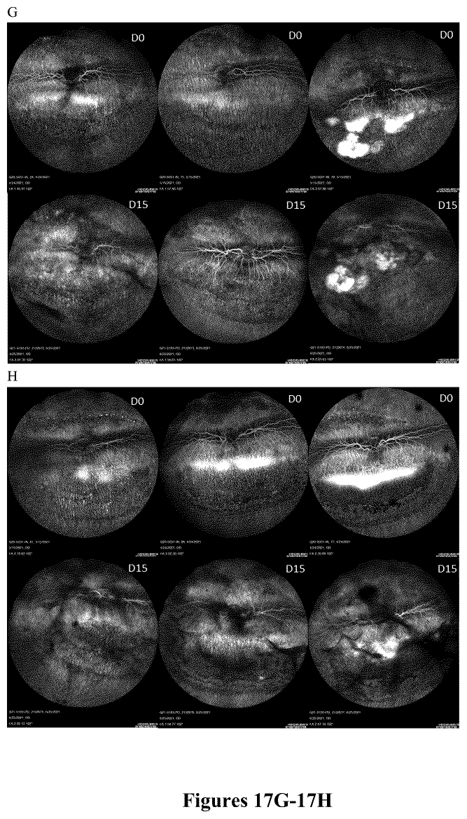

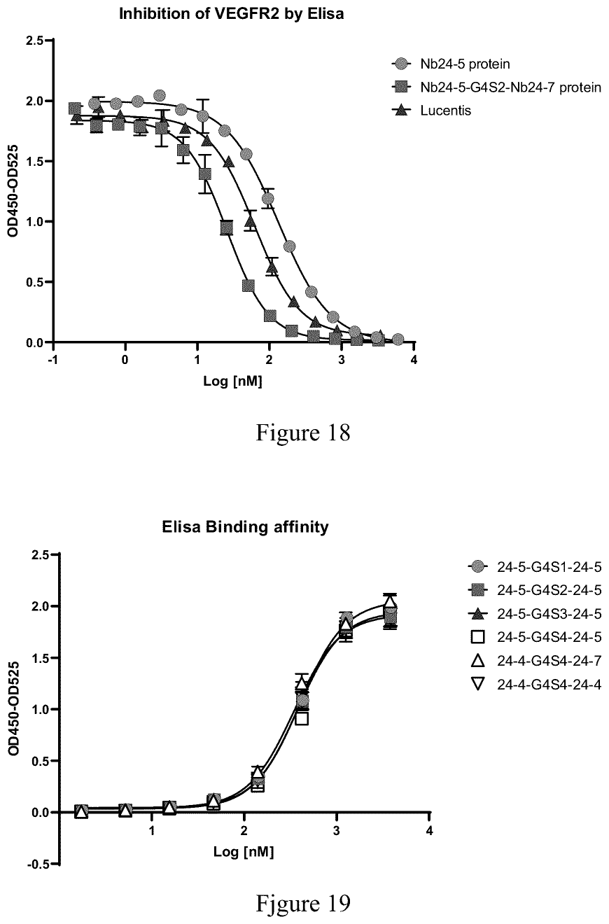

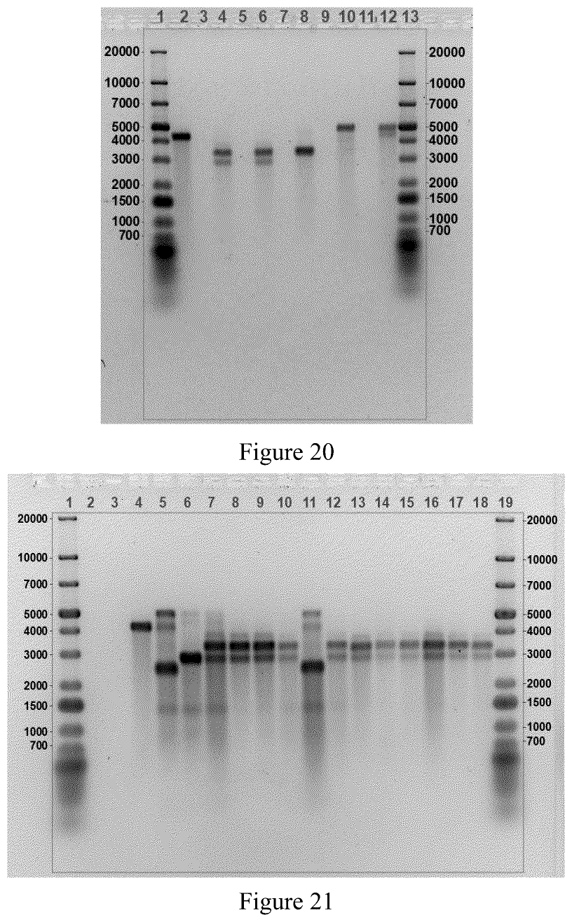

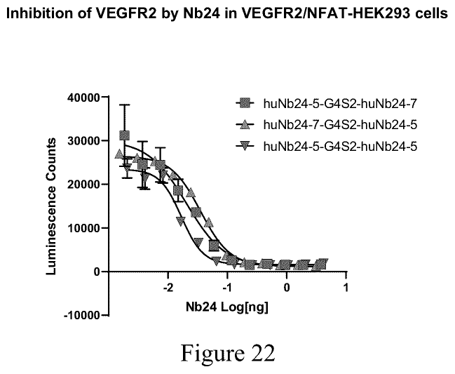

As shown in FIG. 7A, all the 11 codon optimized plasmids (pGCB108-ITR130-AMDFINb24-n, n=1-11) can efficiently express Nb24. The expressions of the plasmids were calculated based on the intensity of the signal, and normalized based on the expression by pGCB108-ITR130-AMDF1Nb24-11, which was indicated as “1”. The higher expressions were observed in three plasmids (pGCB108-ITR130-AMDF1Nb24-4, -5 and -7), while the expression by pGCB108-ITR130-AMDF1Nb24-5 was the highest, more than two times higher than pGCB108-ITR130-AMDF1Nb24-11 (FIG. 7B).

### Example 3. Expression of Bi-Valent Nb24 Nanobody

3.1. The Expi293 cells (Thermo) were cultured and transfected with the following plasmids according to manufactory's protocol:
   pGCB108-ITR130-AMDF1 Bi-huNb24-pCDNA3.1+-HA_IL2sig;
   pGCB108-ITR130-AMDF1 Bi-huNb24-hlgG1Fc;
   pGCB108-ITR130-AMDF1Co3.1Nb24(IL2 signal)-HA;
   pGCB108-ITR130-AMDF1Co3.1Nb24(IL2 signal)-hIgG1Fc;
   pGCB108-ITR130-AMDF1Nb24-4(IL2 signal)-HA;
   pGCB108-ITR130-AMDF1Nb24-2; and
   pGCB108-ITR130-AMDF1Nb24-4.

The cell supernatants were collected 72h after transfection, and tested by Western blotting. The cell culturing and transfection, and the Western blotting were carried out as described in Example 2 except the dilution of the primary antibody (1:2000), and that the plasmids encoding a hIgG1Fc was detected by incubating with Anti-Fc HRP (1:3000, invitrogen, A18823) at RT for 2h, and visualized using Odyssey Infrared Imager (Li-Cor).

As shown in FIG. 8, the Bi-valent plasmids (the lanes indicated by 1, 2, 4 and 6) achieved higher expression levels as compared to the mono-valent plasmids (the lanes indicated by 3, 5, 7 and 8).
3.2. The Expi293 cells were cultured and transfected with the following plasmids according to manufactory's protocol:
   pGCB108-ITR130-AMDF1-Beovu-scFv;
   pGCB108-ITR130-AMDF1 Bi-huNb24-pCDNA3.1+-HA_IL2sig;
   pGCB108-ITR130-AMDF1Nb24-5-HA-CMV-AMDF1-Nb24-5-HA;
   pCB108-5ITR130-CBA-pCI-Nb24-5-hGHpA;
   pGCB108-ITR130-AMDF1Nb24-5-(G4S)4-Nb24-5-HA;
   pGCB108-ITR130-AMDF1huNb24-5-hGHpA; and
   pGCB108-ITR130-AMDF1Nb24-5.

The cell supernatants were collected 72h after transfection, and tested by Western blotting. The cell culturing and transfection, and the Western blotting were carried out as described in Example 2 except the dilution of the secondary antibody (1:2500).

As shown in FIG. 9, the dual promoter plasmid (pGCB108-ITR130-AMDF1Nb24-5-HA-CMV-AMDF1-Nb24-5-HA, lane 3) showed increased Nb24 expression as compared to the mono-promoter one (pGCB108-ITR130-AMDF1 Bi-huNb24-pCDNA3.1+-HA_IL2sig, lane 2).

It was also shown that the plasmid comprising a shorter intron (pCB108-5ITR130-CBA-pCI-Nb24-5-hGHpA, lane 4) showed decreased Nb24 expression.
3.3. The Expi293 cells were cultured and transfected with the plasmids pGCB108-ITR130-AMDF1huNb24-5-hGHpA and pGCB108-ITR130-AMDF1Nb24-5-(G4S)n-Nb24-5-HA according to manufactory's protocol. The cell supernatants were collected 72h after transfection and tested by Western blotting. The cell culturing and transfection, and the Western blotting were carried out as described in Example 2 except the dilution of the secondary antibody (1:2500). The result of the Western blotting is shown in FIG. 10: the expression level of mono-valent Nb24 is lower than bi-valent Nb24, and the bi-valent Nb24s were distinct from each other in size due to the length of the linker, while the expression levels thereof were similar.

### Example 4. The Expressions of Different Versions of Nb24

The Expi293 cells were cultured and transfected with the following plasmids according to manufactory's protocol:
pGCB108-ITR130-AMDF1-Beovu-scFv;
pGCB108-ITR130-AMDF1Nb24-5-(G4S)4-Nb24-5-HA;
pGCB108-ITR130-AMDF1 Bi-huNb24-pCDNA3.1+-HA_IL2sig;
pGCB108-ITR130-AMDF1Nb24-5-HA-CMV-AMDF1-Nb24-5-HA; and
pGCB108-ITR130-AMDF1huNb24-5-hGHpA.

The cell supernatants were collected 72h after transfection and tested by Western blotting. The cell culturing and transfection, and the Western blotting were carried out as described in Example 2 except the dilution of the secondary antibody (1:2500).

As shown in FIG. 11, the transgene in the plasmids were well expressed, the expression level of mono-valent Nb24 is slightly higher with the dual promoter construct, and the bi-valent Nb24 encoded by Nb24-5 (SEQ ID NOs: 5 and 16) was higher than that encoded by Co3.1 HuNb24 (SEQ ID NO: 40).

### Example 5. Binding of HuNb24s to VEGF

The binding of HuNb24 was tested by ELISA with VEGFA-165 (R&D systems 293-VE/CF). In particular, the HuNb24 was expressed by transfecting expi293 cells with the plasmid pGCB108-ITR130-AMDF1huNb24-5-hGHpA and pGCB108-ITR130-AMDF1-Beovu-scFv, and the supernatants of the cultures of the transfected cells (hereinafter referred to as Nb24 and Beovu, respectively) were collected 72h after transfection. The concentrations of the supernatants were determined by Western blotting as described in Example 2. The ELISA was performed as follows, the supernatant from the non-transfected cells was used as negative control.

The ELISA plate (Corning, REF3690) was coated with VEGFA-165 at a concentration of 1 μg/ml, 50 μl/well to half-well high of the plate, overnight. After the incubation with the starter blocker (StartingBlock™ T20 (TBS) Blocking Buffer, Catalog number: 37543 of Thermo Fisher Scientific) at room temperature with gentle shaking for 2 hours, pre-diluted Nb24 supernatant and Beovu supernatant were added to the ELISA plate, followed by the incubation at room temperature with gentle shaking for 2 hours. After removing the blocker, the primary antibody (rabbit anti-HA, Thermo, PA1-985) was diluted (1:5000) with the blocking buffer (StartingBlock™ T20 (TBS) Blocking Buffer, Catalog number: 37543 of Thermo Fisher Scientific), and added to ELISA plate (50 μl/well), followed by the incubation at RT for 1h with 250 rpm gentle shaking. After washing with 1×TBST buffer for 3 times, the secondary antibody (Goat anti-rabbit-HRP, Invitrogen, 3146) was diluted (1:5000) with the blocking buffer, and added to the ELISA plate (50 μl/well), followed by the incubation at RT for 1h with gentle shaking. After washing the ELISA plate with 1×TBST for 3 times, the TMB substrate (Thermo Scientific, REF34028) was added the plate (50 μl/well); after the incubation at RT for 5-10 min, 2M H₂SO₄ was added to stop the TMB reaction. OD450 and OD525 of the ELISA plate were detected. The dose-dependent bindings of Nb24 and Beovu to human VEGFA were calculated using Graph pad prism software.

The following plasmids were also tested for the binding of the expressed mono-valent and bi-valent Nb24s as described above:
pGCB108-ITR130-AMDF1Nb24-5-HA-CMV-AMDF1-Nb24-5-HA;
pGCB108-ITR130-AMDF1Nb24-5-(G4S)n-Nb24-5-HA (n=1-4);
pGCB108-ITR130-AMDF1Nb24-4-(G4S)4-Nb24-4-HA;
pGCB108-ITR130-AMDF1Nb24-4-(G4S)4-Nb24-7-HA;
pGCB108-ITR130-AMDF1Nb24-7-(G4S)4-Nb24-7-HA; and
pGCB108-ITR130-AMDF1 Bi-huNb24-pCDNA3.1+-HA_IL2sig.

As shown in FIG. 12, mono-valent and bi-valent Nb24s bound to VEGF well. Further, the standard curves for mono-valent and various bi-valent Nb24s were in similar shapes (FIGS. 12C and 12D).

### Example 6. Inhibition of VEGFR2 by Nb24

The inhibition of kinase activity of VEGF in VEGFR2/NFAT Reporter-HEK293 cells (BPS Bioscience, Catalog #: 79387) by Nb24 was tested as follows.

VEGFR2/NFAT Reporter-HEK293 cells were cultured in the complete growth medium according to the manufactory's instructions, harvested from the culture, and seeded into a white clear-bottom 96-well microplate (PerkinElmer, 6005) at a density of ˜40,000 cells per well in 100 μl of the complete growth medium. The cells were incubated at 37° C. in a CO₂ incubator for ˜16 hours. The complete growth medium was removed and 80 μl of assay medium (according to the manufactory's instructions) was added. The cells were incubated at 37° C. in a CO₂ incubator for ˜ 1 hours. A mixture of VEGF and mono-valent Nb24 and bi-valent Nb24 was prepared by a) preparing a VEGF solution in assay medium (250 ng/ml); b) preparing serial diluted Nb24 solution in assay medium (with the initial concentration of 10 μg/ml, 2× serial dilution, 12 samples, and the last point with assay medium only); c) 30 μl of the VEGF solution and 30 μl of the serial diluted bi-valent Nb24 solution were mixed, and incubated at room temperature with shaking at 150 rpm for 1 hour. 20 μl of the mixture was added in duplicate to the wells comprising the cells, and 100 μl of assay medium was added to cell-free control wells for determining background luminescence. The plate was incubated at 37° C. in a CO₂ incubator for 4 hours. The luciferase assay was performed using ONE-Step™ Luciferase Assay kit (ONE-Glo™ Luciferase Assay System, Catalog number selected: E6120 of Promega), according to the recommended instructions: adding 100 μl of the final ONE-Step™ Luciferase reagent per well and rocking at room temperature for ˜15 to 30 minutes, and measuring the luminescence using a luminometer.

The inhibitions were presented by the luminescence, which was calculated by subtracting the average background luminescence (cell-free control wells) from the luminescence reading of each of the wells.

As shown in FIG. 13A, the bi-valent Nb24s are similar in inhibiting the kinase activity of VEGF, with an IC 50 from 1.449-2.478 nM (see Table 1 below).

TABLE 1
IC50 of the bi-valent Nb24s

| | Nb24-5-G4S4- | Nb24-4-G4S4- | Nb24-4-G4S4- | Nb24-7-G4S4- | Nb24-5-G4S1- | Nb24-5-G4S2- | Nb24-5-G4S3- |
|---|---|---|---|---|---|---|---|
| | Nb24-5 | Nb24-4 | Nb24-7 | Nb24-7 | Nb24-5 | Nb24-5 | Nb24-5 |
| IC50 | 1.510 | 1.449 | 1.463 | 2.380 | 2.465 | 2.478 | 2.078 |

As shown in FIG. 13B, the bi-valent Nb24 exhibited a stronger inhibition than the mono-valent Nb24.

### Example 7. The Binding of VEGFR2 to VEGF

The binding of human VEGFR2 to human VEGF was detected by ELISA.

The ELISA plate was coated with VEGFA-165 at a concentration of 1 μg/ml, 50 μl/well to half-well high of the plate, overnight. After the incubation with the starter blocker at room temperature with gentle shaking for 2 hours, pre-diluted Human VEGFR2/KDR Fc Chimera Protein (RnD systems and Novamab) was added to ELISA plate, followed by the incubation at room temperature with gentle shaking for 2 hours. After removing the blocker, the detection antibody (rabbit anti-human FC HRP, Thermo) diluted 1:5000 with the blocking buffer, and added 50 μl/well incubated at RT for 1h with 250 rpm gentle shaking. After washing the ELISA plate with 1×TBST for 3 times, the TMB substrate was added the plate (50 μl/well); after the incubation at RT for 5-10 min, 2M H₂SO₄ was added to stop the TMB reaction. OD450 and OD525 of the ELISA plate were detected. The dose-dependent bindings of the VEGFR2 to human VEGF were calculated using Graph pad prism software

As shown in FIG. 14A, VEGFR2 can bind to human VEGF in the absence of Nb24.

A competitive ELISA was also performed according to a similar procedure, while a serial diluted Nb24 or Beovu solution was mixed with the pre-diluted Human VEGFR2/KDR Fc Chimera Protein (1 μg/ml) at a ratio of 1:1 (v/v).

As shown in FIG. 14B, the binding of VEGFR2 to VEGF was inhibited by Nb24 and Beovu.

### Example 8. The Binding of VEGF to VEGFR2

The binding of VEGF to VEGFR2 was detected by a cell based assay as follows.
1) VEGFR2/NFAT Reporter HEK293 Recombinant Cells were cultured according to manufactory's protocol.
2) VEGF dose response to HEK293 Recombinant Cells was performed according to manufactory's (BPS Bioscience) protocol with VEGFA-165.
3) The luciferase assay was performed using ONE-Step™ Luciferase Assay kit.
4) The data were calculated using Graphpad Prism.

As shown in FIG. 15A, the hVEGF exhibited a dose-dependent binding to the cell expressing VEGFR2 in the absence of Nb24 with an EC50 of 17.43 ng/ml.

The assay described in Example 6, which was slightly modified by employing the mixture of VEGF (25 ng/ml) and serial diluted Nb24-5(G4S)4Nb24-5, was performed to detect the effect of Nb24 on the binding of VEGF to VEGFR2. It was shown that the binding of VEGF to VEGFR2 was inhibited by Nb24 with an EC50 of 30.40 ng/ml (FIG. 15B).

### Example 9. Gene Therapy with the Vectors Encoding Nb24

Recombinant AAVs as listed in Table 2 (groups 3-8) were prepared with a method similar to that described in Crosson S M et al. 2018, using different packaging plasmids and transgene plasmids. Briefly, 3E6 cells/ml 293VPC cells (Thermo, Catalog A35347) in serum free virus production medium OPM-293 CD05 (Shanghai OPM Biosciences Co. Ltd. Catalog: 81075-001) were triple transfected, using polyethylenimine, with the helper plasmid, the packaging plasmid encoding rep/cap, and the transgene plasmid below:
helper plasmid (“pHelper” containing the Ad E2A, E4, and VA RNA helper genes as described in Crosson Sm et al., the map thereof is shown in FIG. 16A);
packaging plasmid, an exemplified map is shown in FIG. 16B, in which “AAV2 Cap” can be substituted by the nucleotide sequence encoding the Cap polypeptide of AAV2-m2 (for group 3, SEQ ID NO: 44), AAV9 (for groups 4 and 6, SEQ ID NO: 45), AAV2.GL (for group 5, SEQ ID NO: 46), AAV2-7m8-flank (for group 7, SEQ ID NO: 47), or AAV2-NN (for group 8, SEQ ID NO: 48), and “AAV2 Rep” is the nucleotide sequence encoding the wildtype AAV2 Rep polypeptide;
transgene plasmid, pGCB108-ITR130-AMDF1huNb24-5-hGHpA (for groups 3 and 5-8), or pGCB108-ITR130-AMDF1Nb24-5-(G4S)n-Nb24-5-HA (for group 4).

Following 72 h incubation at 37° C., cells were harvested, and viral particles were purified through an iodixanol gradient (see Crosson S M et al.).

The rAAVs were tested for titers by ddPCR, which were all at the level greater than 1012 viral genomes (vg)/mL (see Table 2). In particular, the ddPCR was carried out with Bio-Rad's QXDx AutoDG ddPCR System and QXDx Universal Kit for AutoDG ddPCR System according to the manufacturer's instructions, and the following primers and probe.

qPCR-AMD-24-5-F
(SEQ ID NO: 50)
GAGTGCGAGCTGGTGAG
qPCR-AMD-24-5-R
(SEQ ID NO: 51)
GCGTAGTCCCTGCTGATG
qPCR-AMD-24-5-P
(SEQ ID NO: 52)
CAAGGACGGCAGCACCTACTACAC

The gene therapy was performed in Dutch belted (DB) rabbit (JOINN New Drug Development Center, Ltd.).

In particular, retinal neovascularization (RNV) was induced unilaterally in the right eye (OD) of two cohorts of Dutch belted (DB) rabbits by intravitreal injection (IVI) of DL-α-AAA in April 2020 and July 2020, respectively. The RNV in the experimental eye was evaluated by fundus imaging and retinal fluorescence angiography (FA) on Apr. 24, and May 16, 2021, respectively and those with visible RNV and fluorescence leakage were selected for gene therapy.

The severity of RNV was numerically graded by measuring the area of the fluorescence leakage (pixels) of the electronic fundus images. The selected animals were grouped (n=3/group) randomly and to ensure there is no significant difference of the leakage areas among the groups (data not shown).

The tested articles listed in Table 2, including controls and rAAVs were administered to the right eyes of the animals by an intravitreal (IVT) injection at a dose of 100 μl/eye. The commercial drug for AMD, Eylea® was used as positive control, while the vehicle was used as negative control. The effects of the tested articles were evaluated by fundus imaging and retinal fluorescence angiography (FA).

TABLE 2
The tested vectors (the titer was identified by ddPCR)

| Group # | Tested Articles | Titer or dose | |
|---|---|---|---|
| 1 | Vehicle | dPBS + 200 mM | |
| | | NaCl + 0.005% PF68* | |
| 2 | Eylea ® | 4 | mg |
| 3 | ssAAV2.m2-Kan-5ITR130- | 8.26E12 | vg/ml |
| | Nb24-5-hGHpA-HA | | |
| 4 | ssAAV9-5ITR130-Nb24-5- | 1.22E13 | vg/ml |
| | G4S4-Nb24-5-hGHpA-HA | | |
| 5 | ssAAV2.GL-5ITR130- | 7.67E12 | vg/ml |
| | Nb24-5HA | | |
| 6 | ssAAV9-5ITR130-Nb24-5- | 1.22E13 vg/ml | |
| | hGHpA-HA | (4.18E13 3.43X) | |
| 7 | ssAAV2-7m8-flank-5ITR130- | 2E12 | vg/ml |
| | Nb24-5-hGHpA-HA | | |
| 8 | ssAAV2-NN-Kan-5ITR130- | 2.12E12 | vg/ml |
| | Nb24-5-hGHpA-HA | | |

*dPBS and PF68 were purchased from Thermo under Catalogs of 10010072 and 24040032, respectively.

It was observed that the vehicle did not alter the RNV induced fluorescein leakage (FIG. 16A), and the inflammatory responses in eyes post IVT were negligible, while Eylea® (4 mg/100 μl) completely stopped the RNV caused leakage (FIG. 16B), and the inflammatory responses in the eyes were mild including cellular infiltration in aqueous humor, vitreal humor and vitreal cloudiness.

In group 3, the RNV caused leakage was not improved 14 days post administration (FIG. 16C), and the severity of inflammatory responses in the eyes was mild and similar to that observed in the Eylea group.

In group 4, a significant therapeutic efficacy on RNV leakage was induced (FIG. 16D), and the inflammatory responses in the eyes were mild to moderate including cellular infiltration in vitreal humor and vitreal cloudiness.

In group 5, a significant therapeutic efficacy on RNV leakage was induced (FIG. 16E), and the inflammatory responses in the eyes were moderate including cellular infiltration in aqueous humor, vitreal humor and obvious vitreal cloudiness.

In group 6, the RNV caused leakage was not improved 14 days post administration (FIG. 16F), and mild to moderate vitreal cellular infiltration and cloudiness were the major observations.

In group 7, the RNV caused leakage was partially improved 14 days post administration (FIG. 16G), and mild cellular infiltration in aqueous humor, vitreal humor and vitreal cloudiness were the major observations.

In group 8, the RNV caused leakage was partially improved 14 days post administration (FIG. 16H), and moderate cellular infiltration in vitreal humor and cloudiness were the major observations.

### Example 10. Optimization of the Constructs encoding Nb24

This Example was performed to obtain an optimal construct encoding Nb24.
10.1 Bi-valent Nb24 achieved a higher inhibition to the VEGFR2 than mono-valent Nb24.

The Expi293 cells were transfected according to Example 2 with plasmids:
pGCB108-ITR130-AMDF1huNb24-5-hGHpA, and
pGCB108-ITR130-AMDF1Nb24-5-(G4S)2-Nb24-7-HA.

The mono-valent and bi-valent Nb24s in the supernatants were quantified by western blot as described in Example 2, and diluted to a series of concentrations. The inhibitions thereof to the VEGFR2 were tested by ELISA as described in Example 7, and the commercial available product Lucentis (NOVARTIS, SAWE7) was used as the reference.

As shown in FIG. 18, bi-valent Nb24 showed an IC50 5× better than mono-valent Nb24 (25.6 nM vs. 132.5 nm), while the Lucentis showed an IC50 of 62.1 nM.
10.2. Bi-valent Nb24s with a linker (G4S)3 or (G4S)2 showed higher affinity than those with (G4S)1 or (G4S)4.

The Expi293 cells were transfected according to Example 2 with plasmids:
pGCB108-ITR130-AMDF1Nb24-5-(G4S)n-Nb24-5-HA (n=1-4);
pGCB108-ITR130-AMDF1Nb24-4-(G4S)4-Nb24-4-HA, and
pGCB108-ITR130-AMDF1Nb24-4-(G4S)4-Nb24-7-HA.

The binding of the bi-valent Nb24s to VEGF was tested as described in Example 5. As shown in FIG. 19, the curves were similar in shape. The bi-valent Nb24s with linkers (G4S)3 and (G4S)2 showed lower EC50 values (345.5 nM and 376.1 nM, respectively) than those with the linkers (G4S)1 and (G4S)4 (400.2 nM and 463.8 nM, respectively).
10.3. rAAVs encoding bi-valent Nb24 with identical coding sequences showed poor virus quality.

rAAVs were prepared by the method as described in Example 9 while the transgene plasmid selected from:
pGCB108-ITR130-AMDF1Nb24-5-(G4S)n-Nb24-5-HA (n=1-4),
pGCB108-ITR130-AMDF1Nb24-5_stuffer,
pGCB108-ITR130-AMDF1Nb24-5-(G4S)4-Nb24-5-HA_stuffer,
pGCB108-ITR130-AMDF1huNb24-5-hGHpA,
pGCB108-ITR130-AMDF1Nb24-5-HA-CMV-AMDF1-Nb24-5-HA,
pGCB108-ITR130-AMDF1Nb24-4-(G4S)4-Nb24-4-HA,
pGCB108-ITR130-AMDF1Nb24-4-(G4S)4-Nb24-7-HA, and
pGCB108-ITR130-AMDF1Nb24-7-(G4S)4-Nb24-7-HA.

The resulted rAAVs were shown in Tables 3 and 4.

The rAAVs were heated at 95° C. for 5 min and loaded to denaturing gel for electrophoresis (20V, overnight, at 4° C.).

TABLE 3
rAAV information for FIG. 20 (lanes 1 and 13: GeneRuler 1 kb Plus
DNA ladder, and lane 2 is a rAAV genome as positive control)

| Lane | Transgene plasmid | Cap |
|---|---|---|
| 4 | pGCB108-ITR130-AMDF1Nb24-4- | AAV9 |
| | (G4S)4-Nb24-4-HA | |
| 6 | pGCB108-ITR130-AMDF1Nb24-7- | AAV9 |
| | (G4S)4-Nb24-7-HA | |
| 8 | pGCB108-ITR130-AMDF1Nb24-4- | AAV9 |
| | (G4S)4-Nb24-7-HA | |
| 10 | pGCB108-ITR130-AMDF1huNb24- | AAV9 |
| | 5-hGHpA | |
| 12 | pGCB108-ITR130-AMDF1Nb24-5- | AAV9 |
| | (G4S)4-Nb24-5-HA_stuffer | |

TABLE 4
rAAV information for FIG. 21 (lanes 1 and 19: GeneRuler 1 kb Plus
DNA ladder, and lane 4 is a rAAV genome as positive control)

| Lane | Transgene plasmid | Cap |
|---|---|---|
| 5 | pGCB108-ITR130-AMDF1Nb24-5- | AAV2-7m8 |
| | HA-CMV-AMDF1-Nb24-5-HA | |
| 6 | pGCB108-ITR130-AMDF1huNb24- | AAV2-7m8 |
| | 5-hGHpA | |
| 7 | pGCB108-ITR130-AMDF1Nb24-5- | AAV2.GL |
| | (G4S)4-Nb24-5-HA | |
| 8 | pGCB108-ITR130-AMDF1Nb24-5- | AAV9 |
| | (G4S)4-Nb24-5-HA | |
| 9 | pGCB108-ITR130-AMDF1Nb24-5- | AAV9 |
| | (G4S)3-Nb24-5-HA | |
| 10 | pGCB108-ITR130-AMDF1huNb24- | AAV2-7m8 |
| | 4-hGHpA | |
| 11 | pGCB108-ITR130-AMDF1Nb24-5- | AAV2-NN |
| | HA-CMV-AMDF1-Nb24-5-HA | |
| 12 | pGCB108-ITR130-AMDF1Nb24-5- | AAV2-NN |
| | (G4S)4-Nb24-5-HA | |
| 13 | pGCB108-ITR130-AMDF1Nb24-5- | AAV9 |
| | (G4S)1-Nb24-5-HA | |
| 14 | pGCB108-ITR130-AMDF1Nb24-5- | AAV2-M2 |
| | (G4S)4-Nb24-5-HA | |
| 15 | pGCB108-ITR130-AMDF1Nb24-5- | AAV2-7m8 |
| | (G4S)4-Nb24-5-HA | |
| 16 | pGCB108-ITR130-AMDF1Nb24-5- | AAV2-M2 |
| | (G4S)3-Nb24-5-HA | |
| 17 | pGCB108-ITR130-AMDF1Nb24-5- | AAV2.GL |
| | (G4S)3-Nb24-5-HA | |
| 18 | pGCB108-ITR130-AMDF1Nb24-5- | AAV9 |
| | (G4S)2-Nb24-5-HA | |

As shown in FIGS. 20 and 21, rAAVs encoding bi-valent Nb24 with identical coding sequences showed two brands in the electrophoresis of the genomes thereof (lanes 4, 6 and 12 in FIG. 20, and lanes 7-9 and 12-18 of FIG. 21), indicating that the corresponding rAAV populations comprise heterogeneous genomes, i.e., indicating a poor virus quality (poor genome integrity), while rAAVs encoding bi-valent Nb24 with different coding sequences showed a single brand (lane 8 of FIG. 20), indicating the homogeneous genome, i.e., an advantageous virus quality (desired genome integrity).

rAAVs were also prepared with the transgene plasmid selected from pGCB108-ITR130-AMDF1Nb24-5-(G4S)2-Nb24-7-HA and pGCB108-ITR130-AMDF1Nb24-7-(G4S)2-Nb24-5-HA, and the packaging plasmid encoding a Cap polypeptide selected from AAV2-7m8 and AAV9, respectively. The denaturing electrophoresis of the genomes isolated from the resulted rAAVs also showed a single brand, indicating the homogeneous genome, i.e., an advantageous virus quantity (desired genome integrity).

It is concluded that the rAAVs encoding bi-valent Nb24 with different coding sequences achieved an advantage over the rAAVs encoding bi-valent Nb24 with identical coding sequences in virus quality.
10.4. Arrangement of the coding sequences for Nb24 in the bi-valent constructs does not significantly affect the expression.

The Expi293 cells were transfected according to Example 2 with plasmids:
pGCB108-ITR130-AMDF1Nb24-5-(G4S)2-Nb24-5-HA,
pGCB108-ITR130-AMDF1Nb24-5-(G4S)2-Nb24-7-HA, and
pGCB108-ITR130-AMDF1Nb24-7-(G4S)2-Nb24-5-HA.

The bi-valent Nb24s in the supernatants were quantified by western blot as described in Example 2, and diluted to a series of concentrations. The inhibitions thereof to the VEGFR2 were tested as described in Example 6. The results were shown in FIG. 22. The bi-valent Nb24s expressed by the three plasmids did not show significant difference from each other in the expression level and the inhibition to VEGFR2.

Sequence listing

SEQ ID NO: 1 (huNb24-1)
ATGAAATACCTACTCCCCACCGCTGCCGCCGGCCTGTTACTGCTCGCGGCGCAGCCTGCCATGGCA
GAGGTGCAATTGCAAGAGAGCGGCGGCGGCCTTGTGCAGCCTGGTGGCAGCCTGAGACTGAGCTGCACCGC
TAGCGGCTTCACCTTCGACGACCCCGACGTGGGCTGGTTCAGACAAGCCCCCGGCAACGAGTGCGAGCTGG
TGAGCACCATCAGCAAGGACGGCAGCACCTACTACACCGACAGCGTGAAGGGCAGATTCACCATCAGCAGA
GACTACGCCAAGAACACCGTGTACCTGCAGATGAACAGCCTGAGAGCCGAGGACACCGCCGTGTACTACTG
CGCCGCCGACAGCAACCCCATCGCCCCCATCAGAACCTGCCTGGGCTGGTACAACTACTGGGGCCAAGGCA
CCCTGGTGACCGTGAGCAGC
SEQ ID NO: 2 (huNb24-2)
ATGAAATACCTGCTGCCTACCGCCGCTGCCGGCCTGCTGCTGCTCGCCGCCCAGCCCGCCATGGCC
GAAGTGCAGCTGCAGGAGAGCGGCGGAGGACTGGTCCAGCCTGGAGGCAGCCTGCGGCTGAGCTGTACAGC
TTCTGGCTTTACCTTCGACGACCCCGACGTGGGCTGGTTCCGGCAGGCCCCTGGCAACGAGTGCGAGCTGG
TGTCCACCATCAGCAAGGACGGCTCTACATACTACACCGACAGCGTGAAGGGCAGATTCACCATCAGCAGA
GATTACGCCAAGAACACCGTGTACCTGCAAATGAACAGCCTGAGAGCCGAAGATACCGCCGTGTATTACTG
CGCCGCTGATTCTAATCCTATCGCCCCAATCAGAACATGCCTGGGCTGGTACAACTACTGGGGCCAGGGCA
CCCTGGTTACAGTGTCCAGC
SEQ ID NO: 3 (huNb24-3)
ATGAAATACCTGCTGCCTACAGCCGCCGCTGGCCTGCTGCTCCTGGCCGCTCAGCCAGCTATGGCC
GAAGTGCAGCTGCAGGAGAGCGGAGGCGGCCTGGTGCAGCCTGGCGGATCTCTGAGACTGTCTTGCACAGC
CAGCGGCTTTACCTTCGACGACCCTGATGTGGGCTGGTTCCGGCAGGCCCCCGGAAATGAGTGCGAGCTGG
TCAGCACCATCAGCAAGGACGGCTCTACCTACTACACCGACTCCGTGAAGGGCAGATTCACCATCAGCAGA
GATTACGCCAAGAACACCGTGTACCTGCAAATGAACAGCCTGCGGGCCGAAGATACCGCCGTGTATTACTG
CGCCGCCGACAGCAACCCCATCGCCCCTATCAGAACATGTCTGGGCTGGTACAACTACTGGGGCCAGGGCA
CCCTGGTGACAGTGTCCAGC
SEQ ID NO: 4 (huNb24-4)
ATGAAATACCTGCTGCCTACAGCCGCCGCTGGCCTGCTGCTCCTGGCCGCTCAGCCAGCTATGGCC
GAGGTGCAGCTGCAGGAGAGCGGCGGCGGCCTGGTGCAGCCCGGCGGCAGCCTGAGGCTGAGCTGCACCGC
CAGCGGCTTCACCTTCGACGACCCCGACGTGGGCTGGTTCAGGCAGGCCCCCGGCAACGAGTGCGAGCTGG
TGAGCACCATCAGCAAGGACGGCAGCACCTACTACACCGACAGCGTGAAGGGCAGGTTCACCATCAGCAGG
GACTACGCCAAGAACACCGTGTACCTGCAGATGAACAGCCTGAGGGCCGAGGACACCGCCGTGTACTACTG
CGCCGCCGACAGCAACCCCATCGCCCCCATCAGGACCTGCCTGGGCTGGTACAACTACTGGGGCCAGGGCA
CCCTGGTGACCGTGAGCAGC
SEQ ID NO: 5 (huNb24-5)
ATGAAGTACCTGCTGCCCACCGCCGCCGCCGGCCTGCTGCTGCTGGCCGCCCAGCCCGCCATGGCC
GAGGTGCAGCTGCAGGAGAGCGGCGGCGGCCTGGTGCAGCCCGGCGGCAGCCTGAGGCTGAGCTGCACCGC
CAGCGGCTTCACCTTCGACGACCCCGACGTGGGCTGGTTCAGGCAGGCCCCCGGCAACGAGTGCGAGCTGG
TGAGCACCATCAGCAAGGACGGCAGCACCTACTACACCGACAGCGTGAAGGGCAGGTTCACCATCAGCAGG
GACTACGCCAAGAACACCGTGTACCTGCAGATGAACAGCCTGAGGGCCGAGGACACCGCCGTGTACTACTG
CGCCGCCGACAGCAACCCCATCGCCCCCATCAGGACCTGCCTGGGCTGGTACAACTACTGGGGCCAGGGCA
CCCTGGTGACCGTGAGCAGC
SEQ ID NO: 6 (huNb24-6)
ATGAAGTACCTGCTGCCTACAGCCGCTGCTGGACTGCTGCTTCTTGCTGCTCAACCTGCCATGGCC
GAGGTCCAGCTTCAAGAATCTGGCGGAGGACTGGTTCAGCCTGGCGGATCTCTGAGACTGAGCTGTACCGC
CAGCGGCTTCACCTTCGACGATCCTGATGTCGGCTGGTTCAGACAGGCCCCTGGAAACGAGTGTGAACTGG
TGTCCACCATCAGCAAGGACGGCAGCACCTACTACACCGACAGCGTGAAGGGCAGATTCACCATCTCCAGA
GACTACGCCAAGAACACCGTGTACCTGCAGATGAACAGCCTGAGAGCCGAGGACACCGCCGTGTATTATTG
TGCCGCCGACAGCAACCCTATCGCTCCCATCAGAACATGCCTCGGCTGGTACAACTATTGGGGCCAGGGCA
CACTGGTCACCGTGTCATCT
SEQ ID NO: 7 (huNb24-7)
ATGAAGTACTTGCTGCCCACTGCTGCGGCTGGCCTGCTCCTTCTGGCGGCTCAACCTGCCATGGCG
GAAGTGCAACTGCAGGAGTCCGGTGGGGGTCTGGTTCAGCCCGGCGGATCCCTGCGGCTTAGCTGTACCGC
GTCCGGTTTCACCTTCGATGACCCTGACGTGGGCTGGTTCCGTCAGGCCCCAGGCAACGAGTGTGAACTGG
TGTCCACCATCTCCAAGGATGGGTCCACCTACTATACCGATTCTGTGAAAGGCCGCTTTACCATCAGTCGC
GATTACGCTAAGAATACCGTGTACTTGCAGATGAATTCCCTCCGTGCCGAGGACACCGCGGTTTACTATTG
TGCCGCAGACAGCAACCCTATTGCTCCCATCAGAACCTGCCTTGGTTGGTACAATTACTGGGGTCAGGGAA
CCTTGGTCACAGTGTCTAGC
SEQ ID NO: 8 (huNb24-8)
ATGAAATACCTGTTGCCCACTGCAGCCGCTGGACTGCTCCTGTTGGCTGCCCAGCCCGCGATGGCC
GAGGTACAGCTTCAGGAATCCGGCGGTGGCCTTGTGCAACCCGGTGGCTCTTTGCGGCTGAGCTGCACTGC
TTCTGGCTTTACCTTCGATGACCCAGACGTTGGATGGTTTCGGCAGGCCCCGGGTAACGAGTGTGAACTGG
TGTCTACCATCAGTAAAGACGGCAGCACATACTATACAGATAGCGTTAAAGGCAGGTTTACCATTTCACGT
GACTACGCCAAGAACACAGTGTATCTGCAGATGAACAGCTTGAGGGCGGAGGATACAGCTGTCTATTACTG
TGCAGCTGACTCTAACCCGATCGCGCCCATCCGGACGTGTCTGGGCTGGTATAACTATTGGGGCCAGGGCA
CGCTCGTTACTGTGTCATCC
SEQ ID NO: 9 (huNb24-9)
ATGAAGTACCTCCTGCCTACCGCTGCGGCCGGACTGCTTCTCTTGGCCGCTCAGCCGGCTATGGCA
GAGGTCCAACTGCAAGAGAGCGGCGGAGGCCTGGTGCAGCCAGGGGGTTCTCTCAGGCTGTCATGCACAGC
GAGCGGATTCACATTTGACGATCCTGATGTAGGCTGGTTCAGGCAGGCCCCTGGAAATGAATGTGAGTTGG
TCAGCACTATCAGCAAGGATGGTTCTACATATTACACTGACTCTGTAAAGGGCAGATTCACTATTAGTAGG
GATTATGCGAAGAACACCGTTTACCTTCAGATGAACAGTCTGCGCGCAGAGGACACTGCAGTGTATTACTG
CGCTGCCGACTCCAATCCAATCGCTCCCATTCGTACATGCCTTGGCTGGTACAATTATTGGGGCCAGGGAA
CCTTGGTGACCGTGAGCAGC
SEQ ID NO: 10 (huNb24-10)
ATGAAGTACCTGCTGCCTACAGCCGCTGCTGGACTGCTGCTTCTTGCTGCTCAACCTGCCATGGCC
GAGGTCCAGCTTCAAGAATCTGGCGGAGGACTGGTTCAGCCTGGCGGATCTCTGAGACTGAGCTGTACCGC
CAGCGGCTTCACCTTCGACGATCCTGATGTCGGCTGGTTCAGACAGGCCCCTGGAAACGAGTGTGAACTGG
TGTCCACCATCAGCAAGGACGGCAGCACCTACTACACCGACAGCGTGAAGGGCAGATTCACCATCTCCAGA
GACTACGCCAAGAACACCGTGTACCTGCAGATGAACAGCCTGAGAGCCGAGGACACCGCCGTGTATTATTG
TGCCGCCGACAGCAACCCTATCGCTCCCATCAGAACATGCCTCGGCTGGTACAACTATTGGGGCCAGGGCA
CACTGGTCACCGTGTCATCT
SEQ ID NO: 11 (huNb24-11)
ATGAAATACTTGCTTCCCACGGCAGCTGCCGGACTCCTCCTGTTGGCTGCGCAACCCGCGATGGCT
GAAGTTCAACTCCAGGAATCTGGGGGAGGTCTCGTACAGCCTGGAGGCAGTTTGAGGCTGTCTTGTACTGC
GAGTGGCTTTACATTTGACGATCCCGACGTTGGGTGGTTTCGGCAAGCCCCGGGGAATGAATGTGAGCTTG
TCAGTACTATATCCAAGGACGGTAGCACGTACTACACAGACTCCGTGAAGGGCAGATTTACAATTAGCAGG
GACTATGCTAAGAATACCGTGTATTTGCAGATGAACAGTCTCAGGGCGGAGGATACAGCTGTTTATTACTG
TGCAGCAGACTCTAATCCGATTGCGCCGATTCGGACATGCCTGGGCTGGTATAACTATTGGGGTCAAGGCA
CGCTTGTCACTGTTAGCAGT
SEQ ID NO: 12 (huNb24-1 no signal)
GAGGTGCAATTGCAAGAGAGCGGCGGCGGCCTTGTGCAGCCTGGTGGCAGCCTGAGACTGAGCTGCACCGC
TAGCGGCTTCACCTTCGACGACCCCGACGTGGGCTGGTTCAGACAAGCCCCCGGCAACGAGTGCGAGCTGG
TGAGCACCATCAGCAAGGACGGCAGCACCTACTACACCGACAGCGTGAAGGGCAGATTCACCATCAGCAGA
GACTACGCCAAGAACACCGTGTACCTGCAGATGAACAGCCTGAGAGCCGAGGACACCGCCGTGTACTACTG
CGCCGCCGACAGCAACCCCATCGCCCCCATCAGAACCTGCCTGGGCTGGTACAACTACTGGGGCCAAGGCA
CCCTGGTGACCGTGAGCAGC
SEQ ID NO: 13 (huNb24-2 no signal)
GAAGTGCAGCTGCAGGAGAGCGGCGGAGGACTGGTCCAGCCTGGAGGCAGCCTGCGGCTGAGCTGTACAGC
TTCTGGCTTTACCTTCGACGACCCCGACGTGGGCTGGTTCCGGCAGGCCCCTGGCAACGAGTGCGAGCTGG
TGTCCACCATCAGCAAGGACGGCTCTACATACTACACCGACAGCGTGAAGGGCAGATTCACCATCAGCAGA
GATTACGCCAAGAACACCGTGTACCTGCAAATGAACAGCCTGAGAGCCGAAGATACCGCCGTGTATTACTG
CGCCGCTGATTCTAATCCTATCGCCCCAATCAGAACATGCCTGGGCTGGTACAACTACTGGGGCCAGGGCA
CCCTGGTTACAGTGTCCAGC
SEQ ID NO: 14 (huNb24-3 no signal)
GAAGTGCAGCTGCAGGAGAGCGGAGGCGGCCTGGTGCAGCCTGGCGGATCTCTGAGACTGTCTTGCACAGC
CAGCGGCTTTACCTTCGACGACCCTGATGTGGGCTGGTTCCGGCAGGCCCCCGGAAATGAGTGCGAGCTGG
TCAGCACCATCAGCAAGGACGGCTCTACCTACTACACCGACTCCGTGAAGGGCAGATTCACCATCAGCAGA
GATTACGCCAAGAACACCGTGTACCTGCAAATGAACAGCCTGCGGGCCGAAGATACCGCCGTGTATTACTG
CGCCGCCGACAGCAACCCCATCGCCCCTATCAGAACATGTCTGGGCTGGTACAACTACTGGGGCCAGGGCA
CCCTGGTGACAGTGTCCAGC
SEQ ID NO: 15 (huNb24-4 no signal)
ATGAAATACCTGCTGCCTACAGCCGCCGCTGGCCTGCTGCTCCTGGCCGCTCAGCCAGCTATGGCC
GAGGTGCAGCTGCAGGAGAGCGGCGGCGGCCTGGTGCAGCCCGGCGGCAGCCTGAGGCTGAGCTGCACCGC
CAGCGGCTTCACCTTCGACGACCCCGACGTGGGCTGGTTCAGGCAGGCCCCCGGCAACGAGTGCGAGCTGG
TGAGCACCATCAGCAAGGACGGCAGCACCTACTACACCGACAGCGTGAAGGGCAGGTTCACCATCAGCAGG
GACTACGCCAAGAACACCGTGTACCTGCAGATGAACAGCCTGAGGGCCGAGGACACCGCCGTGTACTACTG
CGCCGCCGACAGCAACCCCATCGCCCCCATCAGGACCTGCCTGGGCTGGTACAACTACTGGGGCCAGGGCA
CCCTGGTGACCGTGAGCAGC
SEQ ID NO: 16 (huNb24-5 no signal)
GAGGTGCAGCTGCAGGAGAGCGGCGGCGGCCTGGTGCAGCCCGGCGGCAGCCTGAGGCTGAGCTGCACCGC
CAGCGGCTTCACCTTCGACGACCCCGACGTGGGCTGGTTCAGGCAGGCCCCCGGCAACGAGTGCGAGCTGG
TGAGCACCATCAGCAAGGACGGCAGCACCTACTACACCGACAGCGTGAAGGGCAGGTTCACCATCAGCAGG
GACTACGCCAAGAACACCGTGTACCTGCAGATGAACAGCCTGAGGGCCGAGGACACCGCCGTGTACTACTG
CGCCGCCGACAGCAACCCCATCGCCCCCATCAGGACCTGCCTGGGCTGGTACAACTACTGGGGCCAGGGCA
CCCTGGTGACCGTGAGCAGC
SEQ ID NO: 17 (huNb24-6 no signal)
GAGGTCCAGCTTCAAGAATCTGGCGGAGGACTGGTTCAGCCTGGCGGATCTCTGAGACTGAGCTGTACCGC
CAGCGGCTTCACCTTCGACGATCCTGATGTCGGCTGGTTCAGACAGGCCCCTGGAAACGAGTGTGAACTGG
TGTCCACCATCAGCAAGGACGGCAGCACCTACTACACCGACAGCGTGAAGGGCAGATTCACCATCTCCAGA
GACTACGCCAAGAACACCGTGTACCTGCAGATGAACAGCCTGAGAGCCGAGGACACCGCCGTGTATTATTG
TGCCGCCGACAGCAACCCTATCGCTCCCATCAGAACATGCCTCGGCTGGTACAACTATTGGGGCCAGGGCA
CACTGGTCACCGTGTCATCT
SEQ ID NO: 18 (huNb24-7 no signal)
GAAGTGCAACTGCAGGAGTCCGGTGGGGGTCTGGTTCAGCCCGGCGGATCCCTGCGGCTTAGCTGTACCGC
GTCCGGTTTCACCTTCGATGACCCTGACGTGGGCTGGTTCCGTCAGGCCCCAGGCAACGAGTGTGAACTGG
TGTCCACCATCTCCAAGGATGGGTCCACCTACTATACCGATTCTGTGAAAGGCCGCTTTACCATCAGTCGC
GATTACGCTAAGAATACCGTGTACTTGCAGATGAATTCCCTCCGTGCCGAGGACACCGCGGTTTACTATTG
TGCCGCAGACAGCAACCCTATTGCTCCCATCAGAACCTGCCTTGGTTGGTACAATTACTGGGGTCAGGGAA
CCTTGGTCACAGTGTCTAGC
SEQ ID NO: 19 (huNb24-8 no signal)
GAGGTACAGCTTCAGGAATCCGGCGGTGGCCTTGTGCAACCCGGTGGCTCTTTGCGGCTGAGCTGCACTGC
TTCTGGCTTTACCTTCGATGACCCAGACGTTGGATGGTTTCGGCAGGCCCCGGGTAACGAGTGTGAACTGG
TGTCTACCATCAGTAAAGACGGCAGCACATACTATACAGATAGCGTTAAAGGCAGGTTTACCATTTCACGT
GACTACGCCAAGAACACAGTGTATCTGCAGATGAACAGCTTGAGGGCGGAGGATACAGCTGTCTATTACTG
TGCAGCTGACTCTAACCCGATCGCGCCCATCCGGACGTGTCTGGGCTGGTATAACTATTGGGGCCAGGGCA
CGCTCGTTACTGTGTCATCC
SEQ ID NO: 20 (huNb24-9 no signal)
GAGGTCCAACTGCAAGAGAGCGGCGGAGGCCTGGTGCAGCCAGGGGGTTCTCTCAGGCTGTCATGCACAGC
GAGCGGATTCACATTTGACGATCCTGATGTAGGCTGGTTCAGGCAGGCCCCTGGAAATGAATGTGAGTTGG
TCAGCACTATCAGCAAGGATGGTTCTACATATTACACTGACTCTGTAAAGGGCAGATTCACTATTAGTAGG
GATTATGCGAAGAACACCGTTTACCTTCAGATGAACAGTCTGCGCGCAGAGGACACTGCAGTGTATTACTG
CGCTGCCGACTCCAATCCAATCGCTCCCATTCGTACATGCCTTGGCTGGTACAATTATTGGGGCCAGGGAA
CCTTGGTGACCGTGAGCAGC
SEQ ID NO: 21 (huNb24-10 no signal)
GAGGTCCAGCTTCAAGAATCTGGCGGAGGACTGGTTCAGCCTGGCGGATCTCTGAGACTGAGCTGTACCGC
CAGCGGCTTCACCTTCGACGATCCTGATGTCGGCTGGTTCAGACAGGCCCCTGGAAACGAGTGTGAACTGG
TGTCCACCATCAGCAAGGACGGCAGCACCTACTACACCGACAGCGTGAAGGGCAGATTCACCATCTCCAGA
GACTACGCCAAGAACACCGTGTACCTGCAGATGAACAGCCTGAGAGCCGAGGACACCGCCGTGTATTATTG
TGCCGCCGACAGCAACCCTATCGCTCCCATCAGAACATGCCTCGGCTGGTACAACTATTGGGGCCAGGGCA
CACTGGTCACCGTGTCATCT
SEQ ID NO: 22 (huNb24-11 no signal)
GAAGTTCAACTCCAGGAATCTGGGGGAGGTCTCGTACAGCCTGGAGGCAGTTTGAGGCTGTCTTGTACTGC
GAGTGGCTTTACATTTGACGATCCCGACGTTGGGTGGTTTCGGCAAGCCCCGGGGAATGAATGTGAGCTTG
TCAGTACTATATCCAAGGACGGTAGCACGTACTACACAGACTCCGTGAAGGGCAGATTTACAATTAGCAGG
GACTATGCTAAGAATACCGTGTATTTGCAGATGAACAGTCTCAGGGCGGAGGATACAGCTGTTTATTACTG
TGCAGCAGACTCTAATCCGATTGCGCCGATTCGGACATGCCTGGGCTGGTATAACTATTGGGGTCAAGGCA
CGCTTGTCACTGTTAGCAGT
SEQ ID NO: 23 (Nb24 amino acid sequence)
EVQLQESGGGLVQPGGSLRLSCTASGFTFDDPDVGWFRQAPGNECELVSTISKDGSTYYTDSVKGRFTISR
DYAKNTVYLQMNSLRAEDTAVYYCAADSNPIAPIRTCLGWYNYWGQGTLVTVSS
SEQ ID NO: 24 (pelB signal aa sequence)
MKYLLPTAAAGLLLLAAQPAMA
SEQ ID NO: 25 (CMV IE enhancer in construct 1)
GACATTGATTATTGACTAGTTATTAATAGTAATCAATTACGGGGTCATTAGTTCATAGCCCATATATGGAG
TTCCGCGTTACATAACTTACGGTAAATGGCCCGCCTGGCTGACCGCCCAACGACCCCCGCCCATTGACGTC
AATAATGACGTATGTTCCCATAGTAACGCCAATAGGGACTTTCCATTGACGTCAATGGGTGGAGTATTTAC
GGTAAACTGCCCACTTGGCAGTACATCAAGTGTATCATATGCCAAGTACGCCCCCTATTGACGTCAATGAC
GGTAAATGGCCCGCCTGGCATTATGCCCAGTACATGACCTTATGGGACTTTCCTACTTGGCAGTACATCTA
CGTATTAGTCATCGCTATTAC
SEQ ID NO: 26 (CB promoter)
TGGTCGAGGTGAGCCCCACGTTCTGCTTCACTCTCCCCATCTCCCCCCCCTCCCCACCCCCAATTTTGTAT
TTATTTATTTTTTAATTATTTTGTGCAGCGATGGGGGCGGGGGGGGGGGGGGGGCGCGCGCCAGGCGGGGC
GGGGCGGGGCGAGGGGCGGGGCGGGGCGAGGCGGAGAGGTGCGGCGGCAGCCAATCAGAGCGGCGCGCTCC
GAAAGTTTCCTTTTATGGCGAGGCGGCGGCGGCGGCGGCCCTATAAAAAGCGAAGCGCGCGGCGGGCGG
SEQ ID NO: 27 (CMV promoter)
GTGATGCGGTTTTGGCAGTACACCAATGGGCGTGGATAGCGGTTTGACTCACGGGGATTTCCAAGTCTCCA
CCCCATTGACGTCAATGGGAGTTTGTTTTGGCACCAAAATCAACGGGACTTTCCAAAATGTCGTAATAACC
CCGCCCCGTTGACGCAAATGGGCGGTAGGCGTGTACGGTGGGAGGTCTATATAAGCAGAGCT
SEQ ID NO: 28 (beta-globin intron)
TCGAATCCCGGCCGGGAACGGTGCATTGGAACGCGGATTCCCCGTGCCAAGAGTGACGTAAGTACCGCCTA
TAGAGTCTATAGGCCCACAAAAAATGCTTTCTTCTTTTAATATACTTTTTTGTTTATCTTATTTCTAATAC
TTTCCCTAATCTCTTTCTTTCAGGGCAATAATGATACAATGTATCATGCCTCTTTGCACCATTCTAAAGAA
TAACAGTGATAATTTCTGGGTTAAGGCAATAGCAATATTTCTGCATATAAATATTTCTGCATATAAATTGT
AACTGATGTAAGAGGTTTCATATTGCTAATAGCAGCTACAATCCAGCTACCATTCTGCTTTTATTTTATGG
TTGGGATAAGGCTGGATTATTCTGAGTCCAAGCTAGGCCCTTTTGCTAATCATGTTCATACCTCTTATCTT
CCTCCCACAGCTCCTGGGCAACGTGCTGGTCTGTGTGCTGGCCCATCACTTTGGCAAAGAATT
SEQ ID NO: 29 (pCI, promega chimeric intron)
GTAAGTATCAAGGTTACAAGACAGGTTTAAGGAGACCAATAGAAACTGGGCTTGTCGAGACAGAGAAGACT
CTTGCGTTTCTGATAGGCACCTATTGGTCTTACTGACATCCACTTTGCCTTTCTCTCCACAG
SEQ ID NO: 30 (HA tag)
TACCCATACGACGTGCCCGACTACGCC
SEQ ID NO: 31 (rabbit globin polyA)
GATCTTTTTCCCTCTGCCAAAAATTATGGGGACATCATGAAGCCCCTTGAGCATCTGACTTCTGGCTAATA
AAGGAAATTTATTTTCATTGCAATAGTGTGTTGGAATTTTTTGTGTCTCTCACTCG
SEQ ID NO: 32 (hGH poly A)
GGGTGGCATCCCTGTGACCCCTCCCCAGTGCCTCTCCTGGCCCTGGAAGTTGCCACTCCAGTGCCCACCAG
CCTTGTCCTAATAAAATTAAGTTGCATCATTTTGTCTGACTAGGTGTCCTTCTATAATATTATGGGGTGGA
GGGGGGTGGTATGGAGCAAGGGGCAAGTTGGGAAGACAACCTGTAGGGCCTGCGGGGTCTATTGGGAACCA
AGCTGGAGTGCAGTGGCACAATCTTGGCTCACTGCAATCTCCGCCTCCTGGGTTCAAGCGATTCTCCTGCC
TCAGCCTCCCGAGTTGTTGGGATTCCAGGCATGCATGACCAGGCTCAGCTAATTTTTGTTTTTTTGGTAGA
GACGGGGTTTCACCATATTGGCCAGGCTGGTCTCCAACTCCTAATCTCAGGTGATCTACCCACCTTGGCCT
CCCAAATTGCTGGGATTACAGGCGTGAACCACTGCTCCCTTCCCTGTCCTT
SEQ ID NO: 33 (5′ ITR130)
CTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCT
SEQ ID NO: 34 (3′ ITR130)
AGGAACCCCTAGTGATGGAGTTGGCCACTCCCTCTCTGCGCGCTCGCTCGCTCACTGAGGCCGGGCGACCA
AAGGTCGCCCGACGCCCGGGCTTTGCCCGGGCGGCCTCAGTGAGCGAGCGAGCGCGCAG
SEQ ID NO: 35 (5′ ITR105)
CTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTG
SEQ ID NO: 36 (G4S4)
GGAGGAGGCGGCTCTGGCGGAGGCGGCTCTGGCGGAGGCGGATCCGGAGGAGGCGGCTCC
SEQ ID NO: 37 (G4S3)
GGAGGAGGCGGCTCTGGCGGAGGCGGCTCCGGAGGAGGCGGCTCC
SEQ ID NO: 38 (G4S2)
GGAGGAGGCGGCTCTGGCGGAGGCGGCTCT
SEQ ID NO: 39 (G4S1)
GGAGGAGGCGGCTCT
SEQ ID NO: 40 (Co3.1 HuNb24)
GAAGTGCAGCTGCAGGAGTCTGGCGGCGGCCTGGTGCAGCCAGGAGGCTCTCTGAGGCTGTCTTGTACCGC
TTCTGGCTTTACATTTGATGATCCTGATGTGGGCTGGTTTAGACAGGCTCCTGGCAATGAGTGTGAGCTGG
TGAGCACCATTAGTAAGGATGGTAGTACATACTACACAGATTCTGTGAAGGGCAGGTTTACTATTTCTAGA
GATTACGCTAAGAACACAGTGTATCTGCAGATGAATAGCCTGAGGGCCGAGGACACAGCCGTGTACTACTG
CGCCGCCGACTCTAACCCTATCGCTCCTATTAGAACCTGCCTGGGCTGGTACAATTACTGGGGACAGGGCA
CACTGGTGACCGTCAGCTCT
SEQ ID NO: 41 (IL2 signal sequence)
ATGTACAGGATGCAACTCCTGTCTTGCATTGCACTAAGTCTTGCACTTGTCACGAATTCG
SEQ ID NO: 42 (hIgG1Fc in construct 20)
GAGCCCAAGTCTTGTGACAAGACACACACCTGTCCCCCCTGCCCTGCTCCAGAGCTGCTGGGCGGCCCCTC
CGTCTTCCTGTTCCCACCTAAGCCTAAGGATACACTGATGATCAGCCGAACCCCTGAGGTGACCTGCGTGG
TGGTGGACGTGTCTCATGAGGATCCTGAGGTGAAGTTTAACTGGTACGTGGACGGAGTGGAGGTGCATAAC
GCCAAGACAAAGCCAAGAGAGGAGCAGTATAACTCAACATACAGAGTGGTCAGCGTGCTGACCGTGCTGCA
CCAGGACTGGCTGAACGGAAAGGAGTACAAGTGCAAGGTGAGTAATAAGGCTCTGCCAGCTCCAATTGAAA
AGACAATTAGTAAGGCTAAGGGACAGCCAAGGGAGCCCCAGGTGTACACACTGCCTCCTTCTAGAGAGGAA
ATGACAAAGAACCAGGTGAGTCTGACCTGTCTGGTGAAGGGATTTTACCCTAGTGATATTGCCGTAGAGTG
GGAATCTAACGGACAACCTGAAAACAACTATAAGACAACACCTCCTGTGCTGGACTCTGACGGATCTTTCT
TTCTGTATAGTAAGCTGACTGTGGATAAGAGTAGGTGGCAGCAGGGCAACGTGTTTTCTTGTAGCGTGATG
CACGAAGCTCTGCATAACCATTATACACAGAAGTCTCTGAGCCTGAGCCCTGGCAAG
SEQ ID NO: 43 (Beovu-scFv comprising pelB signal)
ATGAAATACCTTCTGCCTACCGCCGCTGCTGGCCTGCTGCTGCTGGCCGCCCAGCCTGCTATGGCCGAAAT
CGTGATGACCCAGAGCCCTTCTACACTCTCCGCCTCCGTGGGCGATAGAGTGATCATCACCTGTCAGGCCA
GCGAAATCATCCACAGCTGGCTGGCTTGGTACCAGCAGAAGCCCGGCAAAGCCCCTAAGCTGCTGATCTAC
CTGGCCTCTACCCTGGCCAGCGGCGTGCCTAGCAGGTTCAGCGGCTCCGGAAGCGGCGCTGAGTTCACCCT
GACCATTTCTAGCCTGCAACCCGATGACTTCGCCACCTACTATTGCCAGAACGTGTACCTGGCCTCTACAA
ACGGCGCTAATTTCGGCCAAGGCACAAAGCTGACCGTGCTGGGCGGCGGAGGCGGCAGCGGCGGCGGCGGA
TCCGGCGGCGGCGGCAGCGGCGGCGGAGGTTCTGAGGTGCAGCTGGTCGAGAGCGGCGGCGGACTGGTTCA
GCCTGGCGGAAGCCTGCGGCTGAGCTGCACCGCCAGCGGATTTAGCCTGACCGACTACTACTACATGACAT
GGGTCAGACAGGCTCCTGGCAAGGGCCTGGAGTGGGTGGGCTTCATCGACCCCGACGATGATCCATACTAT
GCCACATGGGCCAAGGGCAGATTTACCATCAGCCGGGACAACTCAAAGAATACCCTGTACCTGCAGATGAA
CAGCCTGAGAGCCGAAGATACAGCCGTGTACTACTGCGCCGGCGGCGACCACAACAGCGGCTGGGGACTGG
ACATCTGGGGCCAGGGAACACTGGTGACCGTGTCCAGC
SEQ ID NO: 44 (AAV2-m2)
MAADGYLPDWLEDTLSEGIRQWWKLKPGPPPPKPAERHKDDSRGLVLPGYKYLGPENGLDKGEPVNEADAA
ALEHDKAYDRQLDSGDNPYLKYNHADAEFQERLKEDTSFGGNLGRAVFQAKKRVLEPLGLVEEPVKTAPGK
KRPVEHSPVEPDSSSGTGKAGQQPARKRLNFGQTGDADSVPDPQPLGQPPAAPSGLGTNTMATGSGAPMAD
NNEGADGVGNSSGNWHCDSTWMGDRVITTSTRTWALPTYNNHLYKQISSQSGASNDNHYFGYSTPWGYFDF
NRFHCHFSPRDWQRLINNNWGFRPKRLNFKLFNIQVKEVTQNDGTTTIANNLTSTVQVFTDSEYQLPYVLG
SAHQGCLPPFPADVFMVPQYGYLTLNNGSQAVGRSSFYCLEYFPSQMLRTGNNFTFSYTFEDVPFHSSYAH
SQSLDRLMNPLIDQYLYYLSRTNTPSGTTTQSRLQFSQAGASDIRDQSRNWLPGPCYEQQRVSKTSADNNN
SEYSWTGATKYHLNGRDSLVNPGPAMASHKDDEEKFFPQSGVLIFGKQGSEKTNVDIEKVMITDEEEIRTT
NPVATEQYGSVSTNLQEGNRQAATADVNTQGVLPGMVWQDRDVYLQGPIWAKIPHTDGHFHPSPLMGGFGL
KHPPPQILIKNTPVPANPSTTFSAAKFASFITQYSTGQVSVEIEWELQKENSKRWNPEIQYTSNYNKSVNV
DFTVDINGVYSEPRPIGTRYLTRNL
SEQ ID NO: 45 (AAV9)
MAADGYLPDWLEDNLSEGIREWWALKPGAPQPKANQQHQDNARGLVLPGYKYLGPGNGLDKGEPVNAADAAALEH
DKAYDQQLKAGDNPYLKYNHADAEFQERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDYQLPYVLGSAHEGCLPPFPADVEMIP
QYGYLTLNDGSQAVGRSSFYCLEYFPSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSEFAWPGASSWALNGRNSLMNPGPAMA
SHKEGEDRFFPLSGSLIFGKQGTGRDNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIKNTPVPADPPTAFNKDKLNSFITQYS
TGQVSVEIEWELQKENSKRWNPEIQYTSNYYKSNNVEFAVNTEGVYSEPRPIGTRYLTRNL
SEQ ID NO: 46 (AAV2.GL)
MAADGYLPDWLEDTLSEGIRQWWKLKPGPPPPKPAERHKDDSRGLVLPGYKYLGPFNGLDKGEPVNEADAAALEH
DKAYDRQLDSGDNPYLKYNHADAEFQERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGTNTMATGSGAPMADNNEGADGVGNSS
GNWHCDSTWMGDRVITTSTRTWALPTYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEYSWTGATKYHLNGRDSLVNPGPAMAS
HKDDEEKFFPQSGVLIFGKQGSEKTNVDIEKVMITDEEEIRTTNPVATEQYGSVSTNLQRGNAAAGLSPPTRAAR
QAATADVNTQGVLPGMVWQDRDVYLQGPIWAKIPHTDGHFHPSPLMGGFGLKHPPPQILIKNTPVPANPSTTESA
AKFASFITQYSTGQVSVEIEWELQKENSKRWNPEIQYTSNYNKSVNVDFTVDINGVYSEPRPIGTRYLTRNL
SEQ ID NO: 47 (AAV2-7m8-flank)
MAADGYLPDWLEDTLSEGIRQWWKLKPGPPPPKPAERHKDDSRGLVLPGYKYLGPENGLDKGEPVNEADAAALEH
DKAYDRQLDSGDNPYLKYNHADAEFQERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGTNTMATGSGAPMADNNEGADGVGNSS
GNWHCDSTWMGDRVITTSTRTWALPTYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEYSWTGATKYHLNGRDSLVNPGPAMAS
HKDDEEKFFPQSGVLIFGKQGSEKTNVDIEKVMITDEEEIRTTNPVATEQYGSVSTNLQRGNLALGETTRPARQA
ATADVNTQGVLPGMVWQDRDVYLQGPIWAKIPHTDGHFHPSPLMGGFGLKHPPPQILIKNTPVPANPSTTFSAAK
FASFITQYSTGQVSVEIEWELQKENSKRWNPEIQYTSNYNKSVNVDFTVDTNGVYSEPRPIGTRYLTRNL
SEQ ID NO: 48 (AAV2-NN)
MAADGYLPDWLEDTLSEGIRQWWKLKPGPPPPKPAERHKDDSRGLVLPGYKYLGPENGLDKGEPVNEADAAALEH
DKAYDRQLDSGDNPYLKYNHADAEFQERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGTNTMATGSGAPMADNNEGADGVGNSS
GNWHCDSTWMGDRVITTSTRTWALPTYNNHLYKQISSQSGASNDNHYFGYSTPWGYFDENRFHCHESPRDWQRLI
NNNWGFRPKRLNFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEYSWTGATKYHLNGRDSLVNPGPAMAS
HKDDEEKFFPQSGVLIFGKQGSEKTNVDIEKVMITDEEEIRTTNPVATEQYGSVSTNLQRGNAAANNPTPSRAAR
QAATADVNTQGVLPGMVWQDRDVYLQGPIWAKIPHTDGHFHPSPLMGGFGLKHPPPQILIKNTPVPANPSTTESA
AKFASFITQYSTGQVSVEIEWELQKENSKRWNPEIQYTSNYNKSVNVDFTVDTNGVYSEPRPIGTRYLTRNL
SEQ ID NO: 49 (alb-stuffer)
TTTCATTGGCTGCCAACCGATTACAAAATTCATAATATTCTTGCCAAAAAATTGTTTGACTAAATCCCTTG
TGTATATTTACCATCTGGAACTGTTTTCTCCACAAGACCTTGTCAATGTATTAAAGTTGTGTAAAACATCT
TATCAAATTTTACATAAGAGTTTAAACGATTTGTACCTTTTATTCATTACCATGGTATTTAACTGTAATTT
CCCACTTGAACATTTTAGTTCTTGGTGTTTGCAATTTAGTTCTCAGTGTTTAGTAATTACTAACCAAACTA
AATACTTTGCTTAGAAATAAGACATACTGAAAGAGTTAAGTCCCAAATTGCATCTATTGCTAATTCCAAGT
GTGCTTTTACCTATAGATAATGAAAAATTTACACACAAGTACTCACGCATGCACACACACATATACATACA
TACATATTTTAGGGCTTCTGTGTCTTTTTTACTATTCCAAAGCCATTCTGTCTGAAAAAGCTGATGTTGCC
TCAGATGCAAACTAAGAAAAAGACATAGCAACAATAACAAGTAAGATAGGACTTAGAAATTCCATGTTCCC
ATTCCTGCTGTGGCCCACATGTGATTCAAGCCCTGAGATAGGCTATTTAGATTCAGTGTGAGTATATCTCA
TGGGATCTTCAGTTAAGCAGAGATGGAGAGGAAAATGGACCCAAATGAGATGATGATTATCTTTGCCATAA
TATACAATAGGACAGGCTGACCCCAAATTCTCTGTAGGTTATGAGAACCCTTGACCTTCACCAGGCATAGG
ATTCTTTCCATCCCTGTTTCATATTTTTTTCCTTTCTCTCTTTCAATAAGTAGAAAAATCACATGTTCTTT
TATGTTGCTTTAAATTTCAAAATAAATTTAACATTATGTCTAAAAACAAAGCGAAGGAAGAATAATTTTTT
AAATATTTCATAAATCTTCCTTCCCTACCCAAGGTTCTTATCTCTACCTCAGAAAAGCTATCCTACCTCTG
CCCTTTTGCTCAGAATATGAAGAACTACTGAGCTCAACCAGTAGGAAGAACAACTTTCTCTGATACTACAG
AACAATAATTAGTACTAAGCCAGTTATTTTACTTAATTAAATAACTCAGTCAGTCAAAACCAGTAGTCAAA
TTTCCTCATGGCACATAGAGGCACAGAGACTAACAAAGTATTATTTTTTCTACTCAGTCTTATAATGAAAG
AAAATTTTTTCTATTCAGTCTTACAATGAAAGAAATTATTTTCTATAATCTTATGTTATTTCTTCTTCAGA
AATGGGTACTAATTAACTAATAAAGCACAGAAGAGGAAAAAATCAAAGCTAGATGGATGATAGATACATAG
ATAGATAGGAAGATTTTTTTTAATGACTCAAGGGAGTCCTGAAAGGAAATACCAGACTTGTCTTTCTGACT
ACAGATGAACAGACATAAAACAAGTGTGCCTATTTTCCATTGAAGAAAAACAAGCATTTTTTCTAAAGCCA
GAAAAACCTACATCTGAAAAGAAAAAAGAAACTATTCTAAGTTAAGAAAATAATTCTTTCTTGGATAAATT
ATGTCAACTTTTAACTTCCTAGAAATTGTAGGCATTATTTATTAGAAGTCAGTAACCAACTCTAGCTTTTT
CCTTGTAGAAGTCCCTGGGAAAGGCCTACCCAAACATAAAATCCTTACTGTTGAATACAATTGTTACTAAT
TGACAAATTCAGGCCTTTGTCACTCTCACATATACTTTGTGCATACTTTTTCATTTAACAAATGAAAAGAA
TAATATTAGTCCATTTAGTTTTCACCACAACATTATAATGTAATATATTGTTAAATACAAAGAAACCTAAA
AAAATCTCAAATTCATCATGACTGAATAGAAACTTTGAATTGATTTTCCTATATCTAATGAAGAGCCCTAG
CTTTAAAACAAAATTGATGAAACCTAAAAGAATCTTTTCTTTTTTTTTTTTTTTTTTTGAGATGGAGTCTC
GCTCTTTCGCCCAGACTGGAGTGCAGTGGCACTATCTCGGCTCACTTGCAAGCTCCGCCTCCCGAGTTCAC
GCCATTCTTCTGCCTCAGCCTCCCGAGTAGCTGGGACTACAGGCGCCCGCCATCACGCCCGGCTAATTTTT
TGTATCTTTAGTAGAGACGGGGTTTCACCGTGTGCCAGGGTGGTCTCGATCTCCTGACCTCGTCATCCGCC
CACCTCGGCCTCCCAAAGTGCTGGGATTACAGTCATGAGCCA
SEQ ID NO: 50 primer qPCR-AMD-24-5-F
GAGTGCGAGCTGGTGAG
SEQ ID NO: 51 primer qPCR-AMD-24-5-R
GCGTAGTCCCTGCTGATG
SEQ ID NO: 52 probe qPCR-AMD-24-5-P
CAAGGACGGCAGCACCTACTACAC

1. A polynucleotide construct comprising an expression cassette comprising a coding sequence comprising a first nucleotide sequence selected from SEQ ID NOs: 4, 5 and 7, and a second nucleotide sequence linked to the first nucleotide sequence selected from SEQ ID NOs: 15, 16 and 18 via a linker operably linked to a promoter.

2. The polynucleotide construct of claim 1, wherein the linker is selected from SEQ ID NOs: 37 and 38.

3. The polynucleotide construct of claim 1, wherein the promoter comprises a nucleotide sequence of SEQ ID NO: 26 or 27.

4. The polynucleotide construct of claim 1, wherein the expression cassette comprises an enhancer.

5. The polynucleotide construct of claim 4, wherein the enhancer is upstream of the first promoter.

6. The polynucleotide construct of claim 4, wherein the enhancer is a cytomegalovirus (CMV) early enhancer comprising a nucleotide sequence of SEQ ID NO: 25.

7. The polynucleotide construct of claim 1, wherein the expression cassette comprises a polyadenylation signal sequence downstream of the coding sequence.

8. The polynucleotide construct of claim 7, wherein the polyadenylation signal sequence is selected from SEQ ID NOs: 31 and 32.

9. The polynucleotide construct of claim 1, wherein the expression cassette comprises an intron, preferably upstream of the first nucleotide sequence.

10. The polynucleotide construct of claim 9, wherein the intron is at least 200 nucleotides in length.

11. The polynucleotide construct of claim 9, wherein the intron comprises SEQ ID NO: 28.

12. The polynucleotide construct of claim 1, wherein the construct comprises the genome of a recombinant AAV.

13. The polynucleotide construct of claim 12, wherein the construct comprises 5′ and 3′ inverted terminal repeat (ITR) sequences derived from adeno-associated virus (AAV).

14. The polynucleotide construct of claim 13, wherein the 5′ and 3′ ITRs are AAV ITR130 and/or AAV ITR 105.

15. The polynucleotide construct of claim 14, wherein both the 5′ and 3′ ITRs are AAV ITR130.

16. A recombinant adeno-associated virus (rAAV) comprising a genome comprising the polynucleotide construct of any of claim 1.

17. A pharmaceutical composition comprising the rAAV of claim 16.

18. A method of preventing or treating a disease associated with VEGF, comprising administering the rAAV of claim 16 to a subject in need thereof.

19. The method of claim 18, wherein the disease is Wet-AMD or Diabetic macular edema (DME).

20. The method of claim 19, wherein the rAAV is administered by intravitreal injection.

21. A host cell comprising the construct of claim 1.


SEQUENCE LISTING (52 sequences)

SEQ ID NO: 1 - DNA, 441 bp, synthetic construct
  misc_feature 1..441: note = huNb24-1
  source 1..441: mol_type = other DNA
atgaaatacctactccccaccgctgccgccggcctgttactgctcgcggcgcagcctgccatggcagaggtgcaattgcaagagagcggcggcggccttgtgcagcctggtggcagcctgagactgagctgcaccgctagcggcttcaccttcgacgaccccgacgtgggctggttcagacaagcccccggcaacgagtgcgagctggtgagcaccatcagcaaggacggcagcacctactacaccgacagcgtgaagggcagattcaccatcagcagagactacgccaagaacaccgtgtacctgcagatgaacagcctgagagccgaggacaccgccgtgtactactgcgccgccgacagcaaccccatcgcccccatcagaacctgcctgggctggtacaactactggggccaaggcaccctggtgaccgtgagcagc

SEQ ID NO: 2 - DNA, 441 bp, synthetic construct
  misc_feature 1..441: note = huNb24-2
  source 1..441: mol_type = other DNA
atgaaatacctgctgcctaccgccgctgccggcctgctgctgctcgccgcccagcccgccatggccgaagtgcagctgcaggagagcggcggaggactggtccagcctggaggcagcctgcggctgagctgtacagcttctggctttaccttcgacgaccccgacgtgggctggttccggcaggcccctggcaacgagtgcgagctggtgtccaccatcagcaaggacggctctacatactacaccgacagcgtgaagggcagattcaccatcagcagagattacgccaagaacaccgtgtacctgcaaatgaacagcctgagagccgaagataccgccgtgtattactgcgccgctgattctaatcctatcgccccaatcagaacatgcctgggctggtacaactactggggccagggcaccctggttacagtgtccagc

SEQ ID NO: 3 - DNA, 441 bp, synthetic construct
  misc_feature 1..441: note = huNb24-3
  source 1..441: mol_type = other DNA
atgaaatacctgctgcctacagccgccgctggcctgctgctcctggccgctcagccagctatggccgaagtgcagctgcaggagagcggaggcggcctggtgcagcctggcggatctctgagactgtcttgcacagccagcggctttaccttcgacgaccctgatgtgggctggttccggcaggcccccggaaatgagtgcgagctggtcagcaccatcagcaaggacggctctacctactacaccgactccgtgaagggcagattcaccatcagcagagattacgccaagaacaccgtgtacctgcaaatgaacagcctgcgggccgaagataccgccgtgtattactgcgccgccgacagcaaccccatcgcccctatcagaacatgtctgggctggtacaactactggggccagggcaccctggtgacagtgtccagc

SEQ ID NO: 4 - DNA, 441 bp, synthetic construct
  misc_feature 1..441: note = huNb24-4
  source 1..441: mol_type = other DNA
atgaaatacctgctgcctacagccgccgctggcctgctgctcctggccgctcagccagctatggccgaggtgcagctgcaggagagcggcggcggcctggtgcagcccggcggcagcctgaggctgagctgcaccgccagcggcttcaccttcgacgaccccgacgtgggctggttcaggcaggcccccggcaacgagtgcgagctggtgagcaccatcagcaaggacggcagcacctactacaccgacagcgtgaagggcaggttcaccatcagcagggactacgccaagaacaccgtgtacctgcagatgaacagcctgagggccgaggacaccgccgtgtactactgcgccgccgacagcaaccccatcgcccccatcaggacctgcctgggctggtacaactactggggccagggcaccctggtgaccgtgagcagc

SEQ ID NO: 5 - DNA, 441 bp, synthetic construct
  misc_feature 1..441: note = huNb24-5
  source 1..441: mol_type = other DNA
atgaagtacctgctgcccaccgccgccgccggcctgctgctgctggccgcccagcccgccatggccgaggtgcagctgcaggagagcggcggcggcctggtgcagcccggcggcagcctgaggctgagctgcaccgccagcggcttcaccttcgacgaccccgacgtgggctggttcaggcaggcccccggcaacgagtgcgagctggtgagcaccatcagcaaggacggcagcacctactacaccgacagcgtgaagggcaggttcaccatcagcagggactacgccaagaacaccgtgtacctgcagatgaacagcctgagggccgaggacaccgccgtgtactactgcgccgccgacagcaaccccatcgcccccatcaggacctgcctgggctggtacaactactggggccagggcaccctggtgaccgtgagcagc

SEQ ID NO: 6 - DNA, 441 bp, synthetic construct
  misc_feature 1..441: note = huNb24-6
  source 1..441: mol_type = other DNA
atgaagtacctgctgcctacagccgctgctggactgctgcttcttgctgctcaacctgccatggccgaggtccagcttcaagaatctggcggaggactggttcagcctggcggatctctgagactgagctgtaccgccagcggcttcaccttcgacgatcctgatgtcggctggttcagacaggcccctggaaacgagtgtgaactggtgtccaccatcagcaaggacggcagcacctactacaccgacagcgtgaagggcagattcaccatctccagagactacgccaagaacaccgtgtacctgcagatgaacagcctgagagccgaggacaccgccgtgtattattgtgccgccgacagcaaccctatcgctcccatcagaacatgcctcggctggtacaactattggggccagggcacactggtcaccgtgtcatct

SEQ ID NO: 7 - DNA, 441 bp, synthetic construct
  misc_feature 1..441: note = huNb24-7
  source 1..441: mol_type = other DNA
atgaagtacttgctgcccactgctgcggctggcctgctccttctggcggctcaacctgccatggcggaagtgcaactgcaggagtccggtgggggtctggttcagcccggcggatccctgcggcttagctgtaccgcgtccggtttcaccttcgatgaccctgacgtgggctggttccgtcaggccccaggcaacgagtgtgaactggtgtccaccatctccaaggatgggtccacctactataccgattctgtgaaaggccgctttaccatcagtcgcgattacgctaagaataccgtgtacttgcagatgaattccctccgtgccgaggacaccgcggtttactattgtgccgcagacagcaaccctattgctcccatcagaacctgccttggttggtacaattactggggtcagggaaccttggtcacagtgtctagc

SEQ ID NO: 8 - DNA, 441 bp, synthetic construct
  misc_feature 1..441: note = huNb24-8
  source 1..441: mol_type = other DNA
atgaaatacctgttgcccactgcagccgctggactgctcctgttggctgcccagcccgcgatggccgaggtacagcttcaggaatccggcggtggccttgtgcaacccggtggctctttgcggctgagctgcactgcttctggctttaccttcgatgacccagacgttggatggtttcggcaggccccgggtaacgagtgtgaactggtgtctaccatcagtaaagacggcagcacatactatacagatagcgttaaaggcaggtttaccatttcacgtgactacgccaagaacacagtgtatctgcagatgaacagcttgagggcggaggatacagctgtctattactgtgcagctgactctaacccgatcgcgcccatccggacgtgtctgggctggtataactattggggccagggcacgctcgttactgtgtcatcc

SEQ ID NO: 9 - DNA, 441 bp, synthetic construct
  misc_feature 1..441: note = huNb24-9
  source 1..441: mol_type = other DNA
atgaagtacctcctgcctaccgctgcggccggactgcttctcttggccgctcagccggctatggcagaggtccaactgcaagagagcggcggaggcctggtgcagccagggggttctctcaggctgtcatgcacagcgagcggattcacatttgacgatcctgatgtaggctggttcaggcaggcccctggaaatgaatgtgagttggtcagcactatcagcaaggatggttctacatattacactgactctgtaaagggcagattcactattagtagggattatgcgaagaacaccgtttaccttcagatgaacagtctgcgcgcagaggacactgcagtgtattactgcgctgccgactccaatccaatcgctcccattcgtacatgccttggctggtacaattattggggccagggaaccttggtgaccgtgagcagc

SEQ ID NO: 10 - DNA, 441 bp, synthetic construct; identical to SEQ ID NO: 6
  misc_feature 1..441: note = huNb24-10
  source 1..441: mol_type = other DNA

SEQ ID NO: 11 - DNA, 441 bp, synthetic construct
  misc_feature 1..441: note = huNb24-11
  source 1..441: mol_type = other DNA
atgaaatacttgcttcccacggcagctgccggactcctcctgttggctgcgcaacccgcgatggctgaagttcaactccaggaatctgggggaggtctcgtacagcctggaggcagtttgaggctgtcttgtactgcgagtggctttacatttgacgatcccgacgttgggtggtttcggcaagccccggggaatgaatgtgagcttgtcagtactatatccaaggacggtagcacgtactacacagactccgtgaagggcagatttacaattagcagggactatgctaagaataccgtgtatttgcagatgaacagtctcagggcggaggatacagctgtttattactgtgcagcagactctaatccgattgcgccgattcggacatgcctgggctggtataactattggggtcaaggcacgcttgtcactgttagcagt

SEQ ID NO: 12 - DNA, 375 bp, synthetic construct
  misc_feature 1..375: note = huNb24-1 no signal
  source 1..375: mol_type = other DNA
gaggtgcaattgcaagagagcggcggcggccttgtgcagcctggtggcagcctgagactgagctgcaccgctagcggcttcaccttcgacgaccccgacgtgggctggttcagacaagcccccggcaacgagtgcgagctggtgagcaccatcagcaaggacggcagcacctactacaccgacagcgtgaagggcagattcaccatcagcagagactacgccaagaacaccgtgtacctgcagatgaacagcctgagagccgaggacaccgccgtgtactactgcgccgccgacagcaaccccatcgcccccatcagaacctgcctgggctggtacaactactggggccaaggcaccctggtgaccgtgagcagc

SEQ ID NO: 13 - DNA, 375 bp, synthetic construct
  misc_feature 1..375: note = huNb24-2 no signal
  source 1..375: mol_type = other DNA
gaagtgcagctgcaggagagcggcggaggactggtccagcctggaggcagcctgcggctgagctgtacagcttctggctttaccttcgacgaccccgacgtgggctggttccggcaggcccctggcaacgagtgcgagctggtgtccaccatcagcaaggacggctctacatactacaccgacagcgtgaagggcagattcaccatcagcagagattacgccaagaacaccgtgtacctgcaaatgaacagcctgagagccgaagataccgccgtgtattactgcgccgctgattctaatcctatcgccccaatcagaacatgcctgggctggtacaactactggggccagggcaccctggttacagtgtccagc

SEQ ID NO: 14 - DNA, 375 bp, synthetic construct
  misc_feature 1..375: note = huNb24-3 no signal
  source 1..375: mol_type = other DNA
gaagtgcagctgcaggagagcggaggcggcctggtgcagcctggcggatctctgagactgtcttgcacagccagcggctttaccttcgacgaccctgatgtgggctggttccggcaggcccccggaaatgagtgcgagctggtcagcaccatcagcaaggacggctctacctactacaccgactccgtgaagggcagattcaccatcagcagagattacgccaagaacaccgtgtacctgcaaatgaacagcctgcgggccgaagataccgccgtgtattactgcgccgccgacagcaaccccatcgcccctatcagaacatgtctgggctggtacaactactggggccagggcaccctggtgacagtgtccagc

SEQ ID NO: 15 - DNA, 441 bp, synthetic construct; identical to SEQ ID NO: 4
  misc_feature 1..441: note = huNb24-4 no signal
  source 1..441: mol_type = other DNA

SEQ ID NO: 16 - DNA, 375 bp, synthetic construct
  misc_feature 1..375: note = huNb24-5 no signal
  source 1..375: mol_type = other DNA
gaggtgcagctgcaggagagcggcggcggcctggtgcagcccggcggcagcctgaggctgagctgcaccgccagcggcttcaccttcgacgaccccgacgtgggctggttcaggcaggcccccggcaacgagtgcgagctggtgagcaccatcagcaaggacggcagcacctactacaccgacagcgtgaagggcaggttcaccatcagcagggactacgccaagaacaccgtgtacctgcagatgaacagcctgagggccgaggacaccgccgtgtactactgcgccgccgacagcaaccccatcgcccccatcaggacctgcctgggctggtacaactactggggccagggcaccctggtgaccgtgagcagc

SEQ ID NO: 17 - DNA, 375 bp, synthetic construct
  misc_feature 1..375: note = huNb24-6 no signal
  source 1..375: mol_type = other DNA
gaggtccagcttcaagaatctggcggaggactggttcagcctggcggatctctgagactgagctgtaccgccagcggcttcaccttcgacgatcctgatgtcggctggttcagacaggcccctggaaacgagtgtgaactggtgtccaccatcagcaaggacggcagcacctactacaccgacagcgtgaagggcagattcaccatctccagagactacgccaagaacaccgtgtacctgcagatgaacagcctgagagccgaggacaccgccgtgtattattgtgccgccgacagcaaccctatcgctcccatcagaacatgcctcggctggtacaactattggggccagggcacactggtcaccgtgtcatct

SEQ ID NO: 18 - DNA, 375 bp, synthetic construct
  misc_feature 1..375: note = huNb24-7 no signal
  source 1..375: mol_type = other DNA
gaagtgcaactgcaggagtccggtgggggtctggttcagcccggcggatccctgcggcttagctgtaccgcgtccggtttcaccttcgatgaccctgacgtgggctggttccgtcaggccccaggcaacgagtgtgaactggtgtccaccatctccaaggatgggtccacctactataccgattctgtgaaaggccgctttaccatcagtcgcgattacgctaagaataccgtgtacttgcagatgaattccctccgtgccgaggacaccgcggtttactattgtgccgcagacagcaaccctattgctcccatcagaacctgccttggttggtacaattactggggtcagggaaccttggtcacagtgtctagc

SEQ ID NO: 19 - DNA, 375 bp, synthetic construct
  misc_feature 1..375: note = huNb24-8 no signal
  source 1..375: mol_type = other DNA
gaggtacagcttcaggaatccggcggtggccttgtgcaacccggtggctctttgcggctgagctgcactgcttctggctttaccttcgatgacccagacgttggatggtttcggcaggccccgggtaacgagtgtgaactggtgtctaccatcagtaaagacggcagcacatactatacagatagcgttaaaggcaggtttaccatttcacgtgactacgccaagaacacagtgtatctgcagatgaacagcttgagggcggaggatacagctgtctattactgtgcagctgactctaacccgatcgcgcccatccggacgtgtctgggctggtataactattggggccagggcacgctcgttactgtgtcatcc

SEQ ID NO: 20 - DNA, 375 bp, synthetic construct
  misc_feature 1..375: note = huNb24-9 no signal
  source 1..375: mol_type = other DNA
gaggtccaactgcaagagagcggcggaggcctggtgcagccagggggttctctcaggctgtcatgcacagcgagcggattcacatttgacgatcctgatgtaggctggttcaggcaggcccctggaaatgaatgtgagttggtcagcactatcagcaaggatggttctacatattacactgactctgtaaagggcagattcactattagtagggattatgcgaagaacaccgtttaccttcagatgaacagtctgcgcgcagaggacactgcagtgtattactgcgctgccgactccaatccaatcgctcccattcgtacatgccttggctggtacaattattggggccagggaaccttggtgaccgtgagcagc

SEQ ID NO: 21 - DNA, 375 bp, synthetic construct; identical to SEQ ID NO: 17
  misc_feature 1..375: note = huNb24-10 no signal
  source 1..375: mol_type = other DNA

SEQ ID NO: 22 - DNA, 375 bp, synthetic construct
  misc_feature 1..375: note = huNb24-11 no signal
  source 1..375: mol_type = other DNA
gaagttcaactccaggaatctgggggaggtctcgtacagcctggaggcagtttgaggctgtcttgtactgcgagtggctttacatttgacgatcccgacgttgggtggtttcggcaagccccggggaatgaatgtgagcttgtcagtactatatccaaggacggtagcacgtactacacagactccgtgaagggcagatttacaattagcagggactatgctaagaataccgtgtatttgcagatgaacagtctcagggcggaggatacagctgtttattactgtgcagcagactctaatccgattgcgccgattcggacatgcctgggctggtataactattggggtcaaggcacgcttgtcactgttagcagt

SEQ ID NO: 23 - protein, 125 aa, synthetic construct
  REGION 1..125: note = Nb24
EVQLQESGGGLVQPGGSLRLSCTASGFTFDDPDVGWFRQAPGNECELVSTISKDGSTYYTDSVKGRFTISRDYAKNTVYLQMNSLRAEDTAVYYCAADSNPIAPIRTCLGWYNYWGQGTLVTVSS

SEQ ID NO: 24 - protein, 22 aa, synthetic construct
  REGION 1..22: note = pelB signal aa sequence
MKYLLPTAAAGLLLLAAQPAMA

SEQ ID NO: 25 - DNA, 376 bp, synthetic construct
  misc_feature 1..376: note = CMV IE enhancer
  source 1..376: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 26 - DNA, 282 bp, synthetic construct
  misc_feature 1..282: note = CB promoter
  source 1..282: mol_type = other DNA
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 27 - DNA, 204 bp, synthetic construct
  misc_feature 1..204: note = CMV promoter
  source 1..204: mol_type = other DNA
gtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagct

SEQ ID NO: 28 - DNA, 489 bp, synthetic construct
  misc_feature 1..489: note = beta-globin intron
  source 1..489: mol_type = other DNA
tcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaatt

SEQ ID NO: 29 - DNA, 133 bp, synthetic construct
  misc_feature 1..133: note = pCI, promega chimeric intron
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 30 - DNA, 27 bp, synthetic construct
  misc_feature 1..27: note = HA tag
  source 1..27: mol_type = other DNA
tacccatacgacgtgcccgactacgcc

SEQ ID NO: 31 - DNA, 127 bp, synthetic construct
  misc_feature 1..127: note = rabbit globin polyA
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 32 - DNA, 477 bp, synthetic construct
  misc_feature 1..477: note = hGH poly A
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 33 - DNA, 130 bp, synthetic construct
  misc_feature 1..130: note = 5 ITR130
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 34 - DNA, 130 bp, synthetic construct
  misc_feature 1..130: note = 3 ITR130
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 35 - DNA, 105 bp, synthetic construct
  misc_feature 1..105: note = 5 ITR105
  source 1..105: mol_type = other DNA
ctgcgcgctcgctcgctcactgaggccgcccgggcaaagcccgggcgtcgggcgacctttggtcgcccggcctcagtgagcgagcgagcgcgcagagagggagtg

SEQ ID NO: 36 - DNA, 60 bp, synthetic construct
  misc_feature 1..60: note = G4S4
  source 1..60: mol_type = other DNA
ggaggaggcggctctggcggaggcggctctggcggaggcggatccggaggaggcggctcc

SEQ ID NO: 37 - DNA, 45 bp, synthetic construct
  misc_feature 1..45: note = G4S3
  source 1..45: mol_type = other DNA
ggaggaggcggctctggcggaggcggctccggaggaggcggctcc

SEQ ID NO: 38 - DNA, 30 bp, synthetic construct
  misc_feature 1..30: note = G4S2
  source 1..30: mol_type = other DNA
ggaggaggcggctctggcggaggcggctct

SEQ ID NO: 39 - DNA, 15 bp, synthetic construct
  misc_feature 1..15: note = G4S1
  source 1..15: mol_type = other DNA
ggaggaggcggctct

SEQ ID NO: 40 - DNA, 375 bp, synthetic construct
  misc_feature 1..375: note = Co3.1 HuNb24
  source 1..375: mol_type = other DNA
gaagtgcagctgcaggagtctggcggcggcctggtgcagccaggaggctctctgaggctgtcttgtaccgcttctggctttacatttgatgatcctgatgtgggctggtttagacaggctcctggcaatgagtgtgagctggtgagcaccattagtaaggatggtagtacatactacacagattctgtgaagggcaggtttactatttctagagattacgctaagaacacagtgtatctgcagatgaatagcctgagggccgaggacacagccgtgtactactgcgccgccgactctaaccctatcgctcctattagaacctgcctgggctggtacaattactggggacagggcacactggtgaccgtcagctct

SEQ ID NO: 41 - DNA, 60 bp, synthetic construct
  misc_feature 1..60: note = IL2 signal sequence
  source 1..60: mol_type = other DNA
atgtacaggatgcaactcctgtcttgcattgcactaagtcttgcacttgtcacgaattcg

SEQ ID NO: 42 - DNA, 696 bp, synthetic construct
  misc_feature 1..696: note = hIgG1Fc in construct 20
  source 1..696: mol_type = other DNA
gagcccaagtcttgtgacaagacacacacctgtcccccctgccctgctccagagctgctgggcggcccctccgtcttcctgttcccacctaagcctaaggatacactgatgatcagccgaacccctgaggtgacctgcgtggtggtggacgtgtctcatgaggatcctgaggtgaagtttaactggtacgtggacggagtggaggtgcataacgccaagacaaagccaagagaggagcagtataactcaacatacagagtggtcagcgtgctgaccgtgctgcaccaggactggctgaacggaaaggagtacaagtgcaaggtgagtaataaggctctgccagctccaattgaaaagacaattagtaaggctaagggacagccaagggagccccaggtgtacacactgcctccttctagagaggaaatgacaaagaaccaggtgagtctgacctgtctggtgaagggattttaccctagtgatattgccgtagagtgggaatctaacggacaacctgaaaacaactataagacaacacctcctgtgctggactctgacggatctttctttctgtatagtaagctgactgtggataagagtaggtggcagcagggcaacgtgttttcttgtagcgtgatgcacgaagctctgcataaccattatacacagaagtctctgagcctgagccctggcaag

SEQ ID NO: 43 - DNA, 819 bp, synthetic construct
  misc_feature 1..819: note = Beovu-scFv comprising pelB signal
  source 1..819: mol_type = other DNA
atgaaataccttctgcctaccgccgctgctggcctgctgctgctggccgcccagcctgctatggccgaaatcgtgatgacccagagcccttctacactctccgcctccgtgggcgatagagtgatcatcacctgtcaggccagcgaaatcatccacagctggctggcttggtaccagcagaagcccggcaaagcccctaagctgctgatctacctggcctctaccctggccagcggcgtgcctagcaggttcagcggctccggaagcggcgctgagttcaccctgaccatttctagcctgcaacccgatgacttcgccacctactattgccagaacgtgtacctggcctctacaaacggcgctaatttcggccaaggcacaaagctgaccgtgctgggcggcggaggcggcagcggcggcggcggatccggcggcggcggcagcggcggcggaggttctgaggtgcagctggtcgagagcggcggcggactggttcagcctggcggaagcctgcggctgagctgcaccgccagcggatttagcctgaccgactactactacatgacatgggtcagacaggctcctggcaagggcctggagtgggtgggcttcatcgaccccgacgatgatccatactatgccacatgggccaagggcagatttaccatcagccgggacaactcaaagaataccctgtacctgcagatgaacagcctgagagccgaagatacagccgtgtactactgcgccggcggcgaccacaacagcggctggggactggacatctggggccagggaacactggtgaccgtgtccagc

SEQ ID NO: 44 - protein, 735 aa, synthetic construct
  REGION 1..735: note = AAV2-m2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYEQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQEGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 45 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - protein, 747 aa, synthetic construct
  REGION 1..747: note = AAV2.GL
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAAAGLSPPTRAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 47 - protein, 745 aa, synthetic construct
  REGION 1..745: note = AAV2-7m8-flank
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 48 - protein, 747 aa, synthetic construct
  REGION 1..747: note = AAV2-NN
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAAANNPTPSRAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 49 - DNA, 2243 bp, synthetic construct
  source 1..2243: mol_type = other DNA
tttcattggctgccaaccgattacaaaattcataatattcttgccaaaaaattgtttgactaaatcccttgtgtatatttaccatctggaactgttttctccacaagaccttgtcaatgtattaaagttgtgtaaaacatcttatcaaattttacataagagtttaaacgatttgtaccttttattcattaccatggtatttaactgtaatttcccacttgaacattttagttcttggtgtttgcaatttagttctcagtgtttagtaattactaaccaaactaaatactttgcttagaaataagacatactgaaagagttaagtcccaaattgcatctattgctaattccaagtgtgcttttacctatagataatgaaaaatttacacacaagtactcacgcatgcacacacacatatacatacatacatattttagggcttctgtgtcttttttactattccaaagccattctgtctgaaaaagctgatgttgcctcagatgcaaactaagaaaaagacatagcaacaataacaagtaagataggacttagaaattccatgttcccattcctgctgtggcccacatgtgattcaagccctgagataggctatttagattcagtgtgagtatatctcatgggatcttcagttaagcagagatggagaggaaaatggacccaaatgagatgatgattatctttgccataatatacaataggacaggctgaccccaaattctctgtaggttatgagaacccttgaccttcaccaggcataggattctttccatccctgtttcatatttttttcctttctctctttcaataagtagaaaaatcacatgttcttttatgttgctttaaatttcaaaataaatttaacattatgtctaaaaacaaagcgaaggaagaataattttttaaatatttcataaatcttccttccctacccaaggttcttatctctacctcagaaaagctatcctacctctgcccttttgctcagaatatgaagaactactgagctcaaccagtaggaagaacaactttctctgatactacagaacaataattagtactaagccagttattttacttaattaaataactcagtcagtcaaaaccagtagtcaaatttcctcatggcacatagaggcacagagactaacaaagtattattttttctactcagtcttataatgaaagaaaattttttctattcagtcttacaatgaaagaaattattttctataatcttatgttatttcttcttcagaaatgggtactaattaactaataaagcacagaagaggaaaaaatcaaagctagatggatgatagatacatagatagataggaagattttttttaatgactcaagggagtcctgaaaggaaataccagacttgtctttctgactacagatgaacagacataaaacaagtgtgcctattttccattgaagaaaaacaagcattttttctaaagccagaaaaacctacatctgaaaagaaaaaagaaactattctaagttaagaaaataattctttcttggataaattatgtcaacttttaacttcctagaaattgtaggcattatttattagaagtcagtaaccaactctagctttttccttgtagaagtccctgggaaaggcctacccaaacataaaatccttactgttgaatacaattgttactaattgacaaattcaggcctttgtcactctcacatatactttgtgcatactttttcatttaacaaatgaaaagaataatattagtccatttagttttcaccacaacattataatgtaatatattgttaaatacaaagaaacctaaaaaaatctcaaattcatcatgactgaatagaaactttgaattgattttcctatatctaatgaagagccctagctttaaaacaaaattgatgaaacctaaaagaatcttttctttttttttttttttttttgagatggagtctcgctctttcgcccagactggagtgcagtggcactatctcggctcacttgcaagctccgcctcccgagttcacgccattcttctgcctcagcctcccgagtagctgggactacaggcgcccgccatcacgcccggctaattttttgtatctttagtagagacggggtttcaccgtgtgccagggtggtctcgatctcctgacctcgtcatccgcccacctcggcctcccaaagtgctgggattacagtcatgagcca

SEQ ID NO: 50 - DNA, 17 bp, synthetic construct
  source 1..17: mol_type = other DNA
gagtgcgagctggtgag

SEQ ID NO: 51 - DNA, 18 bp, synthetic construct
  source 1..18: mol_type = other DNA
gcgtagtccctgctgatg

SEQ ID NO: 52 - DNA, 24 bp, synthetic construct
  source 1..24: mol_type = other DNA
caaggacggcagcacctactacac